(19) United States
(12) Patent Application Publication
(10) Pub. No.: US 2025/0143713 A1
(43) Pub. Date: May 8, 2025

(54) DEVICES, SYSTEMS, AND METHODS FOR TREATING THE LEFT ATRIAL APPENDAGE

(71) Applicant: Laminar, Inc., Santa Rosa, CA (US)

(72) Inventors: Randall T. Lashinski, Windsor, CA (US); Joshua J. Dwork, Santa Rosa, CA (US); Finn Olavi Rinne, Santa Rosa, CA (US); Padraig J. Savage, Santa Rosa, CA (US); Estella JingYe Wong, Santa Rosa, CA (US)

(21) Appl. No.: 18/999,771

(22) Filed: Dec. 23, 2024

Related U.S. Application Data
(63) Continuation-in-part of application No. 18/153,311, filed on Jan. 11, 2023.
(60) Provisional application No. 63/479,171, filed on Jan. 9, 2023.
(60) Provisional application No. 63/298,928, filed on Jan. 12, 2022.

(51) Int. Cl. A61B 17/12; A61B 90/00

(52) U.S. Cl. CPC A61B 17/12122; A61B 17/12131; A61B 90/37; A61B 2090/376

(57) ABSTRACT

A method includes inserting a contact member into an anatomical cavity. The contact member is rotated within the anatomical cavity through a first range of angular motion and engages tissue of the anatomical cavity during such rotation, such that rotation of the contact member within the anatomical cavity through the first range of angular motion causes torsional deformation of the anatomical cavity. The contact member is moved longitudinally through a first range of longitudinal motion after rotating the contact member within the anatomical cavity through the first range of angular motion, thereby causing longitudinal deformation of the anatomical cavity. A position of the contact member is secured relative to the tissue, thereby maintaining the torsional and longitudinal deformation of the tissue.

### PRIORITY

The present application is a continuation-in-part of U.S. patent application Ser. No. 18/153,311, filed on Jan. 11, 2023, titled DEVICES, SYSTEMS, AND METHODS FOR TREATING THE LEFT ATRIAL APPENDAGE, the content of which is hereby incorporated by reference herein, in its entirety, as if fully set forth herein, for all purposes.

U.S. patent application Ser. No. 18/153,311 claims priority from U.S. Patent Application No. 63/298,928, filed on Jan. 12, 2022, titled LEFT ATRIAL APPENDAGE DEVICE AND TECHNIQUES DEVICES, SYSTEMS, AND METHODS FOR TREATING THE LEFT ATRIAL APPENDAGE and from U.S. Patent Application No. 63/479,171, filed on Jan. 9, 2023, titled DEVICES, SYSTEMS, AND METHODS FOR TREATING THE LEFT ATRIAL APPENDAGE, the contents of each of these priority applications are hereby incorporated by reference herein, in their entirety, as if fully set forth herein, for all purposes.

### FIELD

Arrangements of the present disclosure relate to devices, apparatuses, and methods for closing or occluding a left atrial appendage.

### BACKGROUND

Left atrial appendage (LAA) closure has been typically performed in high-risk patients due to possible stroke risk. LAA closure techniques are generally performed to block emboli from exiting the LAA. Typical surgical closure includes stitching the opening closed via left atrium entry. Other techniques include the application of external clamps such as ATRICLIP manufactured by Atricure where a Nitinol device is used to clamp the appendage without opening the left atrium to exclude the appendage from left atrium blood circulation.

Other solutions have used a plug to close the appendage from the inside of the left atrium. Such plugs can be constructed from a laser cut Nitinol tube expanded to a semi-spherical shape. The portion exposed to the left atrium can be covered with cover-such as a thin micron membrane made from polyethylene terephthalate. The membrane can act as a blood barrier to prevent flow from flowing through and between one or more struts of the plug. Typical sizes range between approximately 20 mm and 35 mm in diameter and approximately 20 mm and 40 mm in depth. The device can have anchors protruding from an outer surface of the device intended to engage the wall of the appendage and prevent movement post deployment. The device can be delivered via venous access through the groin and a transseptal crossing into the left atrium where a guide catheter and coaxial delivery catheter are positioned proximal to the left atrial appendage. The implant for appendage exclusion is typically positioned at the distal most portion of the delivery catheter. The device is typically positioned and deployed using fluoroscopy and echocardiography for guidance. Typical issues with conventional devices include complicated pre-procedural sizing algorithms used to determine the appropriate device size, migration of the implant, leakage around or through the implant, and/or fracture of the implant, all which may exacerbate the thrombus and stroke problem the device was designed to reduce. A typical drug regimen associated with conventional LAA treatment devices includes warfarin anticoagulation for 45 days (approximately 6 weeks) followed by dual antiplatelet therapy (DAPT) for six months post-procedure and aspirin thereafter. Another procedure typically required with conventional LAA treatment devices includes a follow up transesophageal echogram at six weeks following the procedure. The incidence of device-related thrombus in patients with LAA imaging has been reported to be 7.2% per year.

### SUMMARY OF SOME EXEMPLIFYING ARRANGEMENTS

The systems, methods and devices of this disclosure each have several innovative aspects, implementations, or aspects, no single one of which is solely responsible for the desirable attributes disclosed herein.

Disclosed herein are arrangements of a system for treating a left atrial appendage. In some arrangements, the system can include an implant configured to be at least partially positioned within the left atrial appendage, and a catheter configured to deploy the implant, wherein the first dial is configured to axially move the first connector and the outer sheath in a first axial direction from an initial axial position to a second axial position when the first dial is rotated in a first direction, and the second dial is configured to rotate the inner catheter member and the implant in a first rotational direction from an initial position to a second rotational position when the second dial is rotated in a first direction. In some arrangements, the catheter can include an outer sheath coupled with a first connector, a first dial coupled with the outer sheath, an inner catheter member configured to rotate the implant when a distal end portion of the inner tube is engaged with the implant, and a second dial coupled with the inner catheter member.

Any arrangements of the devices, systems, and methods disclosed herein can include, in additional arrangements, one or more of the following features, components, and/or details, in any combination with any of the other features, components, and/or details of any other arrangements disclosed herein: wherein the system is configured to cause a rotating portion of the implant to move from a first state to a second, expanded state and to move an outside surface of the rotating portion of the implant against an inner wall surface of the left atrial appendage; further including a second inner catheter member and a third dial coupled with the second inner catheter member, wherein the third dial is configured to rotate the second inner catheter member and to move a locking portion of the implant toward a rotating portion of the implant when the third dial is rotated in a first direction; wherein the locking portion comprises one or more arms extending away from a body portion of the implant and configured to penetrate into the tissue of the left atrial appendage that has constricted and gathered inwardly around an outside surface of a portion of the implant; wherein the locking portion comprises one or more arms extending away from a body portion of the locking portion, the locking portion being configured to move at least from a first state in which the one or more arms are collapsed and a second state in which the one or more arms are expanded such that an end portion of each of the one or more arms are spaced further apart from the body portion of the implant when the locking portion is in the second state than when the locking portion is in the first state; wherein the one or more arms of the locking portion are configured to extend toward the rotating portion of the implant when the locking portion is in the second state; further comprising a stop element configured to limit a range of movement of the first connector relative to the first dial so as to limit a range of movement of the outer sheath relative to the implant, wherein the stop element is removable and repositionable to adjust range of movement; further including a third inner catheter member and a fourth dial coupled with the third inner catheter member, wherein the fourth dial is configured to rotate the third inner catheter member and unthread the third inner catheter member from the implant to release the implant from the catheter at least when the fourth dial is rotated in a first direction; further including a second inner catheter member and a third dial coupled with the second inner catheter member, wherein the third dial is configured to rotate the second inner catheter member and to move a locking portion of the implant toward a rotating portion of the implant when the third dial is rotated in a first direction; further including a third inner catheter member and a fourth dial coupled with the third inner catheter member, wherein the fourth dial is configured to rotate the third inner catheter member and unthread the third inner catheter member from the implant to release the implant from the catheter at least when the fourth dial is rotated in a first direction, and a linking element comprising a sleeve configured to selectively key the third dial with the fourth dial so that fourth dial cannot be independently rotated relative to the third dial when the linking element is engaged with the fourth dial and the third dial; and/or further including a removable locking element configured to selectively prevent the linking element from becoming disengaged from the third dial and the fourth dial, wherein the locking element comprises a suture.

Additionally, any arrangements of the devices, systems, and methods disclosed herein can include, in additional arrangements, one or more of the following features, components, and/or details, in any combination with any of the other features, components, and/or details of any other arrangements disclosed herein: wherein the other sheath comprises an inner layer and an outer layer positioned over the inner layer along at least a portion of a length of the inner layer such that a distal end portion of the inner layer having a plurality of openings therein is not covered by the outer layer; wherein the plurality of openings comprise a plurality of angled slits formed in the inner layer of the outer sheath, wherein the plurality of angled slits are configured to increase a flexibility of the outer sheath and to permit a passage of a contrast medium through the angled slits; further including a support stand for supporting the catheter and at least a guide catheter, wherein the support stand is configured to be positioned on a support surface, such as a bed or table, or on a patient's body; and/or wherein the support stand for supporting the catheter comprises a slot therein that is configured to receive a projection of the catheter, and wherein the catheter has a locking element configured to selectively secure the catheter in a desired position along the slot when the locking element is engaged.

Also disclosed herein are arrangements of a method of treating a left atrial appendage, that can include advancing an implant into the left atrial appendage, engaging an inner wall surface of the left atrial appendage with a portion of the implant, rotating the implant in a first direction from an initial position by a first predetermined angle, moving the implant in a proximal direction by a first predetermined distance, and rotating the implant in the first direction by a second predetermined angle.

Additionally, any arrangements of the devices, systems, and methods disclosed herein can include, in additional arrangements, one or more of the following features, components, and/or details, in any combination with any of the other features, components, and/or details of any other arrangements disclosed herein: further including moving the implant in the proximal direction by the first predetermined distance after rotating the implant in the first direction by the first predetermined angle and rotating the implant in the first direction by the second predetermined angle after moving the implant in the proximal direction by the first predetermined distance; further including moving the implant in the proximal direction by the first predetermined distance while simultaneously rotating the implant in the first direction by the first predetermined angle; further including preventing the implant from rotating back to the initial position; further including deploying a securing element to prevent the implant from rotating back to the initial position after rotating the implant in the first direction by the second predetermined angle; wherein rotating the implant in the first direction by the second predetermined angle comprises rotating the implant to a final rotational position, the method further comprising deploying a securing element to prevent the implant from rotating back to the initial position after rotating the implant to the final rotational position; wherein the first predetermined angle is greater than or equal to 90 degrees; wherein the first predetermined angle is greater than or equal to 180 degrees; wherein the first predetermined angle is from 200 degrees to 330 degrees; wherein the first predetermined distance is greater than or equal to 0.5 cm; wherein the first predetermined distance is from 0.25 cm to 1.75 cm; wherein the second predetermined angle is greater than or equal to 15 degrees; wherein the second predetermined angle is greater than or equal to 30 degrees; wherein the second predetermined angle is from 30 degrees to 90 degrees; further including enlarging the implant from a first state to a second state before rotating the implant in the first direction by the first predetermined angle, wherein at least a portion of the implant is enlarged in a radial direction when the implant is in the second state as compared to the first state; and/or further including preventing the implant from rotating back to the first rotational position.

Disclosed herein are arrangements of methods of treating a left atrial appendage. In some arrangements, the method can include twisting the left atrial appendage and/or securing the left atrial appendage in a twisted position. Any arrangements of the methods, devices and systems of treating a left atrial appendage disclosed herein can include, in additional arrangements, one or more of the following steps, features, components, and/or details, in any combination with any of the other steps, features, components, and/or details of any other arrangements disclosed herein: wherein twisting the left atrial appendage can include engaging a wall portion on an inside of the left atrial appendage with a contact member and rotating the contact member from a first rotational position (also referred to herein as a first position) to a second rotational position (also referred to herein as a second position) to twist the left atrial appendage; wherein engaging a wall portion on an inside of the left atrial appendage with a contact member can include advancing a deployment device into the left atrial appendage; wherein engaging a wall portion on an inside of the left atrial appendage can include engaging a wall portion on an inside of the left atrial appendage with one or more tissue anchors; wherein the contact member can be a balloon; wherein the contact member can be positioned on an implant coupled with the deployment device; wherein the implant can be self-expanding, balloon expandable and/or mechanically expandable; further including applying a vacuum through the contact member to engage the left atrial appendage; wherein rotating a component of the deployment device rotates the contact member from the first rotational position to the second rotational position to twist the left atrial appendage; wherein rotating the contact member from the first rotational position to the second rotational position to twist the left atrial appendage can include rotating the contact member at least approximately 90° in either direction from the first rotational position; wherein rotating the contact member from the first rotational position to the second rotational position to twist the left atrial appendage can include rotating the contact member at least approximately 180° in either direction from the first rotational position; wherein rotating the contact member from the first rotational position to the second rotational position to twist the left atrial appendage can include rotating the contact member from approximately 90° to approximately 360° in either direction from the first rotational position; wherein rotating the deployment device can include exerting a torque on the deployment device between 0.25 in-oz of torque and 10 in-oz of torque; wherein the method further includes allowing the contact member to rotate from the second direction to a third rotational position that can be between the first rotational position and the second rotational position (for example, and without limitation, as a result of the tissue relaxing); and/or wherein the contact member includes at least one vacuum port configured to communicate a suction force through the at least one vacuum port from a source of suction.

Further, any arrangements of the methods, devices and systems of treating a left atrial appendage disclosed herein can include, in additional arrangements, one or more of the following steps, features, components, and/or details, in any combination with any of the other steps, features, components, and/or details of any other arrangements disclosed herein: wherein twisting the left atrial appendage can include rotating a portion of the left atrial appendage about an axis from a first rotational position to a second rotational position to twist the left atrial appendage; wherein rotating a portion of the left atrial appendage about an axis from a first rotational position to a second rotational position to twist the left atrial appendage can include rotating the portion of the left atrial appendage at least approximately 90° in either direction from the first rotational position; wherein rotating a portion of the left atrial appendage about an axis from a first rotational position to a second rotational position to twist the left atrial appendage can include rotating the portion of the left atrial appendage at least approximately 180° in either direction from the first rotational position; wherein rotating a portion of the left atrial appendage about an axis from the first rotational position to the second rotational position to twist the left atrial appendage can include rotating the portion of the left atrial appendage from approximately 90° to approximately 360° in either direction from the first rotational position; wherein rotating a portion of the left atrial appendage about an axis from a first rotational position to a second rotational position to twist the left atrial appendage can include twisting the left atrial appendage until an ostium of the LAA can be substantially or completely closed; wherein securing the left atrial appendage in a twisted position can include engaging tissue of the heart that has been twisted; wherein engaging tissue of the heart that has been twisted can include engaging tissue wall with an anchor element; wherein the anchor element can include a suture; wherein securing the left atrial appendage in a twisted position can include securing a tissue of the heart outside of an occluded portion of the left atrial appendage with an anchor element; and/or wherein the anchor element can include a plurality of tissue anchors on at least one surface thereof configured to engage with the internal wall of the heart outside of the left atrial appendage.

Also disclosed herein are arrangements of a method of closing the ostium of a left atrial appendage. In some arrangements, the method can include twisting tissue of the heart to constrict the ostium of the left atrial appendage and/or securing tissue that has gathered as a result of twisting tissue of the heart in a gathered position. Any arrangements of the methods, devices and systems of closing the ostium of a left atrial appendage disclosed herein can include, in additional arrangements, one or more of the following steps, features, components, and/or details, in any combination with any of the other steps, features, components, and/or details of any other arrangements disclosed herein: wherein securing the tissue of the heart in the gathered position can include advancing a securing element into the gathered tissue; wherein the securing element can be a suture; and/or wherein the securing element can be a tissue anchor.

Disclosed herein are arrangements of devices and systems for treating an LAA that can include an implant comprising a contact member, and a securing element, wherein the contact member is configured to rotate at least in a first direction from a first rotational position to a second rotational position, wherein the contact member is configured to twist at least a portion of the LAA when the contact member is rotated from the first rotational position to the second rotational position, and wherein the securing element is configured to prevent a rotation of the implant in a second direction that is opposite to the first direction when the securing element is in an operable state. The contact member can be, in some arrangements, configured to move between a first state and a second state, wherein the contact member is larger or is expanded in the second state. Some arrangements of the contact member can be configured to move from the first state to the second state so that at least a portion of the contact member engages a wall portion of the LAA when the contact member is advanced into the LAA. In any arrangements disclosed herein wherein the contact member moves or expands from a first state to a second state, the contact member can be moved or expanded from the first state to the second state in the left atrium or in the LAA. Further, in any arrangements, the contact member can be configured to remain in a fixed state and/or size during the entire procedure, wherein the contact member can be extended past a distal end of the delivery catheter (or an outside tube of the delivery catheter can be withdrawn) and advanced into contact or engagement with a wall portion of the LAA, and then twisted. This can be done without changing a size of the contact member and/or without expanding the contact member.

Also disclosed herein are arrangements of devices and systems for treating an LAA can include an implant configured to move between a first state and a second state, a catheter configured to advance the implant into the LAA when the implant is in the first state and to cause the implant to move from the first state to the second state so that an outside surface of the implant moves against an inner wall surface of the LAA after the implant has been advanced into the LAA, wherein the catheter is configured to rotate the implant in a first direction from a first rotational position to a second rotational position so that the implant can twist at least a portion of the LAA when the implant is in the second state.

Also disclosed herein are arrangements of devices and systems for drawing a first tissue surface toward a second tissue surface, including a contact member configured to expand from a first state to a second state and a securing element configured to move from a first state to a second state, wherein the contact member can be configured to expand from the first state to the second state so that at least a portion of the contact member engages at least a distal portion of the first tissue surface and at least a distal portion of the second tissue surface, the contact member can be configured to rotate at least in a first direction from a first rotational position to a second rotational position, wherein the rotation of the contact member in the first direction causes at least a proximal portion of the first tissue surface to twist and to move toward a proximal portion of the second tissue surface, and wherein the securing element is configured to prevent a rotation of the implant in a second direction when the securing element is in an operable state and engaged with a tissue portion adjacent to and/or comprising the proximal portions of the first and second tissue surfaces, wherein the second direction is opposite to the first direction. Further, in any device and/or system arrangements disclosed herein, the device can be configured to occlude or close a cavity in a body having the first and second tissue surfaces, the first and second tissue surfaces can be tissue surfaces within any cavity within the body, and/or wherein the rotation of the contact member further causes the proximal portion of the second tissue surface to twist and to move toward the proximal portion of the first tissue surface.

Any arrangements of the devices and systems disclosed herein can include, in additional arrangements, one or more of the following features, components, and/or details, in any combination with any of the other features, components, and/or details of any other arrangements disclosed herein: wherein the implant is self-expandable such that the implant automatically expands from the first state to the second state when a restraint is removed from the implant; wherein the contact member is self-expandable such that at least a portion of the contact member automatically expands from the first state to the second state when a restraint is removed from the contact member; wherein the implant is substantially collapsed when the implant is in the first state and is expanded when the implant is in the second state such that a size of the implant is bigger when the implant is in the second state than when the implant is in the first state; wherein the contact member is biased to remain in the second state after deployment into the LAA; wherein the contact member is configured to be rotated in a clockwise or a counter-clockwise direction; wherein the device is configured to cause a tissue of the left atrium and/or the LAA to constrict around an outer surface of a body portion of the implant when the contact member is rotated to the second rotational position, and the securing element is configured to engage with the tissue that has constricted around the outer surface of the body portion of the implant to prevent rotation of the implant in the second direction; wherein the securing element has a plurality of tissue anchors configured to engage with an internal wall of the heart adjacent to the LAA; wherein the securing element has a helical shape and is configured to rotate about a body portion of the implant during the implantation procedures; wherein the implant is configured to rotate in a first direction from the first rotational position to the second rotational position; wherein the implant is configured to prevent rotation of the implant in a second direction after the implant has been fully deployed, wherein the second direction is opposite to the first direction; wherein the contact member has a plurality of tissue anchors on an outside surface thereof; wherein the plurality of tissue anchors on the outside surface of the contact member are configured to engage an inner wall surface of the LAA after the contact member has been moved to the second state; wherein the implant comprises a securing element configured to engage with a tissue portion of the heart adjacent to the LAA; wherein the second rotational position is at least one-quarter of a complete rotation relative to the first rotational position; wherein the second rotational position is at least one-half of a complete rotation relative to the first rotational position; and/or wherein the second rotational position is from approximately one-quarter of a complete rotation to one or more complete rotations relative to the first rotational position.

Further, any arrangements of the devices and systems disclosed herein can include, in additional arrangements, one or more of the following features, components, and/or details, in any combination with any of the other features, components, and/or details of any other arrangements disclosed herein: further comprising a catheter selectively coupled with the contact member and configured to exert a torque on the contact member to rotate the contact member from the first rotational position until a threshold predetermined torque level is reached; wherein a threshold predetermined torque level is from approximately 0.25 in-oz of torque to approximately 10 in-oz of torque; wherein a threshold predetermined torque level is from approximately 0.5 in-oz of torque to approximately 5 in-oz of torque; further comprising a retention element configured to bias the securing element toward a tissue wall of the LAA; further comprising a retention element configured to bias the securing element toward the contact member; further comprising a retention element configured to couple the securing element with the contact member; wherein the retention element comprises a threaded shaft; wherein the device is configured such that a rotation of the retention element in a first direction causes the securing element to move toward the contact member; wherein the contact member is configured to rotate at least in a first direction from a first rotational position to a second rotational position when a torque is applied to the contact member; wherein the device is configured such that the contact member can be removed from the LAA after the securing element has been deployed to the operable state of the securing element; wherein the device is configured such that the contact member can be removed from the LAA after the securing element has been deployed to the operable state of the securing element, and wherein the securing element is configured to prevent a rotation of the tissue of the left atrium and/or the LAA that has been constricted as a result of the rotation of the contact member from the first rotational position to the second rotational position; wherein only a portion of the securing element extends into the left atrium after deployment of the device, and all other portions of the device are internal to the LAA after deployment of the device; wherein only approximately 10% or less of an overall length of the deployed device extends into the left atrium after deployment of the device; wherein a proximal portion of the contact member is configured to bias any folds or overlapped tissue of the left atrial appendage that have formed around the contact member as a result of the rotation of the contact member in the left atrial appendage to slide off or move proximally away from the contact member, so that only a minimal amount of folds or overlapped tissue, if any, will be formed around the outside of the contact member; wherein the securing element is rotationally fixed to the contact member such that a rotation of the contact member will cause an equal and simultaneous rotation of the securing element; wherein the device is configured for use by a surgical robot device or system; a surgical robotic device, comprising one or more robotic arms and wherein the device of any arrangements disclosed herein is configured for use by the surgical robotic device; wherein the contact member and the securing element are integrally formed and/or monolithically formed; wherein the device is configured to cause a tissue of the left atrium and/or the LAA to constrict around an outer surface of a body portion of the implant when the contact member is rotated to the second rotational position, and the securing element is configured to compress the tissue that has constricted around the outer surface of the body portion of the implant between a distal surface of the securing element and the contact member to prevent rotation of the implant in the second direction.

Some arrangements of devices and systems for closing or occluding a left atrial appendage (LAA) disclosed herein can include an implant configured to move between a first state and a second state and a catheter configured to advance the implant into the left atrial appendage when the implant is in the first state, wherein the implant can be configured to move from the first state to the second state so that at least a portion of the implant engages a wall portion of the left atrial appendage after the implant has been advanced into the left atrial appendage, and wherein the implant can be configured to twist at least a portion of the left atrial appendage when the implant is rotated from a first rotational position to a second rotational position when the implant is in the second state. In any arrangements disclosed herein, the twisting movement or step can be accomplished by a torque catheter.

Any arrangements of the devices and systems disclosed herein can, in additional arrangements, include one or more of the following features or details, in any combination: wherein the implant is configured to automatically rotate from the first rotational position to the second rotational position after the implant is in the second state; wherein the implant can be configured to be triggered or activated to thereafter automatically rotate from the first rotational position to the second rotational position; wherein the device has a spring that is coupled with the implant, the spring being configured to automatically rotate the implant when the spring is released or activated; wherein the implant can be self-expandable such that the implant automatically expands from the first state to the second state when a restraint is removed from the implant; wherein the implant can be self-expandable such that at least a portion of the implant automatically expands from the first state to the second state when the implant is advanced past a distal end of an outer sleeve of the catheter; wherein the implant is substantially collapsed when the implant is in the first state and can be expanded when the implant is in the second state such that a size of the implant can be bigger when the implant is in the second state than when the implant is in the first state; wherein the implant can be biased to remain in the second state after deployment into the left atrial appendage; wherein the implant can be configured to be rotated in a clockwise or a counter-clockwise direction; wherein the implant can include a securing element configured to engage with an internal wall of the heart outside of the left atrial appendage; wherein the implant can include a securing element configured to engage with an internal wall of the heart outside of the left atrial appendage, wherein the securing element has a helical shape and is configured to rotate about a body portion of the implant during the implantation procedures; wherein the implant can include a corkscrew shaped securing element configured to engage with an internal wall of the heart outside of the left atrial appendage; wherein the implant can include a securing element having a corkscrew tissue anchor to engage the internal wall of the heart and/or LAA tissue; wherein the implant can include a securing element having a plurality of tissue anchors configured to engage with an internal wall of the heart adjacent to the left atrial appendage; wherein the implant can be configured to prevent the implant from rotating back to the first rotational position after the implant has been fully deployed; wherein the implant can be configured to rotate in a first direction from the first rotational position to the second rotational position, and the implant can be configured to prevent rotation of the implant in a second direction after the implant has been fully deployed, the second direction being opposite to the first direction.

Any arrangements of the devices and systems disclosed herein can, in additional arrangements, include one or more of the following features or details, in any combination: wherein the implant has a plurality of tissue anchors on an outside surface thereof; wherein the plurality of tissue anchors on the outside surface of the implant configured to engage an inner wall surface of the left atrial appendage after the implant has been moved to the second state; wherein the implant can include a securing element configured to engage with a tissue portion of the heart adjacent to the left atrial appendage; wherein the second rotational position can be at least one-quarter or approximately one-quarter of a complete rotation (i.e., 90° or approximately 90°) relative to the first rotational position; wherein the second rotational position can be at least one-half or approximately one-half of a complete rotation (i.e., 180° or approximately 180°) relative to the first rotational position; wherein the second rotational position can be from one-quarter or approximately one-quarter of a complete rotation (i.e., 90° or approximately 90°) to one or more or approximately one or more complete rotations (i.e., 360° or approximately 360° or more) relative to the first rotational position; wherein the catheter can be configured to exert a torque on the implant to rotate the implant from the first rotational position until a threshold predetermined torque level is reached; wherein a threshold predetermined torque level can be from 0.25 or approximately 0.25 in-oz of torque to 10 or approximately 10 in-oz of torque; and/or wherein a threshold predetermined torque level can be from 0.5 or approximately 0.5 in-oz of torque to 5 or approximately 5 in-oz of torque.

Any arrangements of the devices and systems disclosed herein can include an implant having a contact member configured to move between a first state and a second state and a catheter configured to advance the contact member into the LAA when the contact member is in the first state and to cause the contact member to move from the first state to the second state so that an outside surface of the contact member expands against an inner wall surface of the LAA after the contact member has been advanced into the LAA, wherein the catheter is configured to exert a torque on the contact member when at least a portion of the catheter is rotated until a predetermine torque level is reached to rotate the contact member from a first rotational position to a second rotational position so that the contact member can twist at least a portion of the LAA.

Any arrangements of the devices and systems disclosed herein can include an expandable implant configured to move between a first state and a second state, a catheter configured to advance the implant into the left atrial appendage when the implant is in the first state and to cause the implant to move from the first state to the second state so that an outside surface of the implant expands against at least a portion of an inner wall surface of the left atrial appendage after the implant has been advanced into the left atrial appendage. In any arrangements of the device for closing or occluding an LAA disclosed herein, the catheter can be configured to exert a torque on the implant to rotate the implant from a first rotational position to a second rotational position so that the implant can twist at least a portion of the left atrial appendage until a predetermine torque level is reached, or in some arrangements, until the user decides to stop, whichever comes first.

Also disclosed herein are devices and systems for treating the LAA, which include a device configured to be inserted into the LAA and to engage the LAA tissue while the device is rotated to a rotated position to close the blood communication between the LAA and the left atrium. In any arrangements of the apparatus, the device can be configured to be selectively lockable in the rotated position to at least substantially maintain the device in the rotated position after implantation, the device can include a securing element configured to engage a tissue surface adjacent to the LAA to maintain the device in the rotated position after implantation, the device can be round, spherical, or disc shaped when the device is in a deployed state in the LAA, the device can be expandable from a first collapsed state to a second expanded state, and/or the device can be self-expanding from a first collapsed state to a second expanded state.

Also disclosed herein are arrangements of methods for treating the LAA, including engaging a tissue of the LAA, and rotating the tissue of the LAA to close or occlude a blood communication between the LAA and a left atrium. In any arrangements of the methods disclosed herein, rotating the tissue of the LAA to close or occlude the blood communication between the LAA and the left atrium can include rotating the tissue of the LAA to close or occlude the ostium of the LAA. Further, any arrangements of the methods disclosed herein can further include securing the LAA in a rotated position to hold the LAA in a closed or occluded state.

Any arrangements of a method of closing or occluding an LAA disclosed herein can include advancing a deployment device having an implant into the left atrial appendage, wherein the implant can be configured to be moved from a first state to a second state. In some arrangements, at least a portion of the implant can be enlarged in a radial direction when the implant is in the second state as compared to the first state. The method can further include moving the implant from the first state to the second state within the left atrial appendage so as to move at least a portion of an outside wall of the implant or one or more tissue anchors extending away from an outer surface of the implant against at least a portion of an inner wall surface of the left atrial appendage, rotating the implant from a first rotational position to a second rotational position to twist the left atrial appendage, and preventing the implant from rotating back to the first rotational position.

Any arrangements of methods of closing or occluding an LAA disclosed herein can, in some additional arrangements, include one or more of the following steps, in any combination and in any combination with any of the other steps, features, or other details of any other arrangements: wherein the implant is self-expanding and wherein moving the implant from the first state to the second state comprises advancing the implant out of a distal end of the deployment device; wherein engaging a wall portion on an inside of the LAA comprises engaging a wall portion on an inside of the LAA with one or more tissue anchors positioned on an outside surface of the implant; wherein preventing the implant from rotating back to the first rotational position comprises engaging a tissue wall with an anchor element to prevent relative movement between the implant and the tissue wall; wherein preventing the implant from rotating back to the first rotational position comprises engaging a tissue wall with an anchor element, and wherein the anchor element is configured to be secured to the implant to prevent a rotation between the implant and the anchor element; wherein preventing the implant from rotating back to the first rotational position comprises engaging a tissue wall of the heart with an anchor element, wherein the anchor element is rotationally fixed relative to the implant and configured to prevent the implant from rotating back to the first rotational position; wherein preventing the implant from rotating back to the first rotational position comprises engaging a tissue of the heart outside of the closed portion of the LAA with an anchor element, wherein the anchor element is rotationally fixed relative to the implant and configured to prevent the implant from rotating back to the first rotational position; wherein the anchor element comprises a plurality of tissue anchors on at least one surface thereof configured to engage with the internal wall of the heart outside of the LAA; wherein rotating the implant from the first rotational position to the second rotational position to twist the LAA comprises rotating the implant until an ostium of the LAA is substantially or completely closed; wherein rotating the implant from the first rotational position to the second rotational position to twist the LAA comprises rotating the implant at least approximately 90° in either direction from the first rotational position; wherein rotating the implant from the first rotational position to the second rotational position to twist the LAA comprises rotating the implant at least approximately 180° in either direction from the first rotational position; wherein rotating the implant from the first rotational position to the second rotational position to twist the LAA comprises rotating the implant from approximately 90° to approximately 360° in either direction from the first rotational position; wherein rotating the implant from the first rotational position to the second rotational position to twist the LAA comprises rotating the implant from approximately 90° to approximately 180° in either direction from the first rotational position; wherein rotating the implant from the first rotational position to the second rotational position to twist the LAA comprises exerting a torque on the implant to rotate the implant in either direction from the first rotational position until a threshold predetermined torque level is reached, holding the implant in the second rotational position, and securing the implant in approximately the second rotational position relative to a tissue surface surrounding the LAA; wherein a maximum predetermined torque level is from approximately 0.25 in-oz of torque to approximately 10 in-oz of torque; and/or wherein a maximum predetermined torque level is from approximately 0.5 in-oz of torque to approximately 5 in-oz of torque.

Any arrangements of the methods of closing or occluding an LAA disclosed herein can, in some additional arrangements, include one or more of the following steps, in any combination and in any combination with any of the other steps, features, or other details of any other arrangements: wherein the implant is self-expanding and wherein moving the implant from the first state to the second state can include advancing the implant out of a distal end of the deployment device; wherein engaging a wall portion on an inside of the left atrial appendage can include engaging at least a portion of a wall portion on an inside of the left atrial appendage or surrounding the left atrial appendage with one or more tissue anchors positioned on an outside surface of the implant; wherein preventing the implant from rotating back to the first rotational position can include engaging a tissue wall outside of the left atrial appendage with an anchor element; wherein the anchor element can be rotationally fixed to the implant to prevent relative movement between the anchor element and the implant; wherein preventing the implant from rotating back to the first rotational position can include engaging a tissue wall of the heart with an anchor element; wherein the anchor element can be rotationally fixed relative to the implant and configured to prevent the implant from rotating back to the first rotational position; wherein preventing the implant from rotating back to the first rotational position can include engaging an internal wall of the heart outside of the left atrial appendage with an anchor element; wherein the anchor element can be rotationally fixed relative to the implant and configured to prevent the implant from rotating back to the first rotational position; wherein the anchor element can include a plurality of tissue anchors on at least one surface thereof configured to engage with the internal wall of the heart outside of the left atrial appendage; and/or wherein rotating the implant from the first rotational position to the second rotational position to twist the left atrial appendage can include rotating the implant until an ostium of the LAA can be substantially or completely closed or occluded, or collapsed about an outer surface of the implant.

Any arrangements of the methods of closing or occluding an LAA disclosed herein can, in any additional arrangements, include one or more of the following steps, in any combination and in any combination with any of the other steps, features, or other details of any other arrangements: wherein rotating the implant from the first rotational position to the second rotational position to twist the left atrial appendage can include rotating the implant at least one-quarter or approximately one-quarter of a complete rotation (i.e., 90° or approximately 90°) relative to the first rotational position; wherein rotating the implant from the first rotational position to the second rotational position to twist the left atrial appendage can include rotating the implant at least one-half or approximately one-half of a complete rotation (i.e., 180° or approximately 180°) in either direction from the first rotational position; wherein rotating the implant from the first rotational position to the second rotational position to twist the left atrial appendage can include rotating the implant from one-quarter or approximately one-quarter of a complete rotation (i.e., 90° or approximately 90°) to one full turn or approximately one full turn (i.e., 360° or approximately 360°), or to more than one full turn (i.e., more than 360°) in either direction from the first rotational position; wherein rotating the implant from the first rotational position to the second rotational position to twist the left atrial appendage can include rotating the implant from one-quarter or approximately one-quarter of a complete rotation (i.e., 90° or approximately 90°) to one-half of a full turn or approximately one-half of a full turn (i.e., 180° or approximately 180°), or to more than one full turn (i.e., more than 360°) in either direction from the first rotational position; wherein rotating the implant from the first rotational position to the second rotational position to twist the left atrial appendage can include exerting a torque on the implant to rotate the implant in either direction from the first rotational position until a threshold predetermined torque level is reached; wherein rotating the implant from the first rotational position to the second rotational position to twist the left atrial appendage can include holding the implant in the second rotational position; wherein rotating the implant from the first rotational position to the second rotational position to twist the left atrial appendage can include securing the implant in approximately the second rotational position relative to a tissue surface surrounding the left atrial appendage; wherein a maximum predetermined torque level can be from approximately 0.25 in-oz of torque to approximately 10 in-oz of torque; and/or wherein a maximum predetermined torque level can be from approximately 0.5 in-oz of torque to approximately 5 in-oz of torque.

Some arrangements of an implant for deployment within a cavity or vessel disclosed herein include an expandable body, a plurality of tissue anchors on an outside surface of the expandable body configured to engage with an inner wall surface of the cavity or vessel, and an anchor element coupled with the expandable body configured to engage with a tissue surface adjacent to the inner wall surface of the cavity or vessel.

Any arrangements of the devices and systems disclosed herein can include an expandable implant having a plurality of tissue anchors on an outside surface thereof, the expandable implant being configured to move between a first state in which the implant is substantially collapsed and a second state in which at least a portion of the implant is expanded, and a catheter configured to advance the implant into the left atrial appendage when the implant is in the first state and to cause the implant to move from the first state to the second state so that at least some of the plurality of tissue anchors engage an inner wall surface of the left atrial appendage after the implant has been advanced into the left atrial appendage. In some arrangements, the catheter can be configured to rotate the implant in a first direction from a first rotational position to a second rotational position so that the implant can twist the wall of the left atrial appendage.

Some arrangements of the devices and systems for closing or occluding an LAA disclosed herein can include an implant configured to move between a first state and a second state, and a catheter configured to advance the implant into the left atrial appendage when the implant is in the first state and to cause the implant to move from the first state to the second state so that an outside surface of the implant moves against an inner wall surface of the left atrial appendage after the implant has been advanced into the left atrial appendage. In some arrangements, the catheter can be configured to rotate the implant in a first direction from a first rotational position to a second rotational position so that the implant can twist at least a portion of the left atrial appendage when the implant is in the second state.

Any arrangements of the methods of treating the left atrial appendage disclosed herein can include engaging a tissue of the left atrial appendage and rotating the tissue of the left atrial appendage to close or significantly close, or inhibit or substantially inhibit, a blood communication between the left atrial appendage and a left atrium. Any arrangements of the method(s) disclosed herein can include, in additional arrangements, one or more of the following features, components, steps, and/or details, in any combination with any of the other features, components, steps, and/or details of any other treatment method arrangements disclosed herein: further including rotating the tissue of the left atrial appendage to close the blood communication between the left atrial appendage and the left atrium can include rotating the tissue of the left atrial appendage to close the ostium of the left atrial appendage, and/or further including securing the left atrial appendage in a rotated position to hold the left atrial appendage in a closed state.

Some arrangements of apparatuses for treating the left atrial appendage disclosed herein can include a device configured to be inserted into the left atrial appendage and to engage the left atrial appendage tissue while the device is rotated to a rotated position to close the blood communication between the left atrial appendage and the left atrium. In some arrangements, the device can be configured to be locked in the rotated position to maintain the device in the rotated position after implantation, wherein the device can include a securing element configured to engage a tissue surface adjacent to the left atrial appendage to maintain the device in the rotated position after implantation, wherein the device can be round, spherical, or disc shaped when the device is in a deployed state in the left atrial appendage, wherein the device can be expandable from a first collapsed state to a second expanded state, and/or wherein the device can be self-expanding from a first collapsed state to a second expanded state.

Disclosed herein are arrangements of devices for treating a left atrial appendage that include an implant having a contact member and a catheter configured to advance the contact member into the left atrial appendage and to cause the contact member to move against an inner wall surface of the left atrial appendage, wherein the catheter is configured to exert a torque on the contact member when at least a portion of the catheter is rotated until a predetermine torque level is reached to rotate the contact member from a first rotational position to a second rotational position so that the contact member can twist at least a portion of the left atrial appendage. In any arrangements disclosed herein, the contact member can be configured to be moved against the inner wall surface of the left atrial appendage without changing a state or shape of the contact member, and/or the contact member can be configured to be movable or expandable from a first state to a second state.

Disclosed herein are arrangements of devices for reducing an opening of the left atrial appendage that include a contact member and a securing element, wherein the contact member is configured to engage a tissue surface of the left atrial appendage, the contact member is configured to rotate at least a portion of the left atrial appendage in a first direction from a first rotational position to a second rotational position and to cause the opening of the left atrial appendage to reduce in size from a first size to a second size, and/or the securing element is configured to engage with at least a portion of tissue adjacent to the opening of the left atrial appendage and to prevent the opening of the left atrial appendage from expanding to the first size. In any arrangements disclosed herein, the contact member can be configured to engage a tissue surface on an outside surface of the left atrial appendage. Further, in any arrangements disclosed herein, the contact member can be configured to engage the tissue surface of the left atrial appendage without changing a state or shape of the contact member.

Any arrangements of the devices disclosed herein can include, in additional arrangements, one or more of the following features, components, and/or details, in any combination with any of the other features, components, and/or details of any other arrangements disclosed herein: wherein the device further includes a delivery catheter; wherein the device further includes an implant of any of the implant arrangements disclosed herein that is advanceable through the delivery catheter when the implant is in a first state; wherein the implant includes a first stage portion and a second stage portion that are each independently deployable to at least a second operable or deployed state; wherein the first stage portion is configured to be at least partially deployed before a second stage portion is deployed; wherein the first stage portion is configured to be positioned near a distal end portion of the LAA; wherein the second stage portion is configured to constrict an opening of the LAA when the second stage portion is in the second state; wherein second stage portion is configured to close the opening of the LAA when the second stage portion is in the second state; wherein second stage portion is configured to fold one or more tissue portions surrounding or adjacent to the opening of the LAA when the second stage portion is in the second state; wherein the second stage portion is configured to twist one or more portions of tissue surrounding the opening of the LAA to constrict or close the opening of the LAA when the second stage portion is in a second state; wherein the second stage portion comprises a means for constricting or closing the opening of the LAA; wherein the second stage portion comprises a hinge mechanism for constricting or closing the opening of the LAA; further including at least one of a passive activation mechanism and an active activation mechanism to activate the hinge mechanism; and/or wherein at least one of the first stage portion and the second stage portion is self-expanding.

Disclosed herein are additional arrangements of treatment methods that include advancing a deployment device having an implant into the left atrial appendage, moving at least a portion of an outside surface of the implant or one or more tissue anchors extending away from an outer surface of the implant against an inner wall surface of the left atrial appendage, rotating the implant from a first rotational position to a second rotational position to twist the left atrial appendage, and preventing the implant from rotating back to the first rotational position. In any arrangements, the method can include moving at least a portion of an outside surface of the implant or one or more tissue anchors extending away from an outer surface of the implant against an inner wall surface of the left atrial appendage without changing a shape or size of the implant, and/or moving the implant from a first state to a second state, and wherein at least a portion of the implant is enlarged in a radial direction when the implant is in the second state as compared to the first state.

Disclosed herein are additional arrangements of devices and systems for closing an LAA that can include a clamp device having a first member and a second member and be configured to move between a closed position and an open position, a first guide device configured to be advanceable into the LAA, and a second guide device configured to be advanceable into a pericardial space outside of the LAA and moved so that an end portion of the second guide device is in approximate axial alignment with an end portion of the first guide device. In any arrangements disclosed herein, at least one of the first and second members of the clamp device can be substantially rigid; the clamp device can have an opening sized so that the clamp device can be passed over the LAA when the clamp device is in the open position; and/or at least one of the first and second members of the clamp device can be configured to substantially flatten and close a portion of the LAA when the clamp device is moved to the closed position. In any additional arrangements disclosed herein, the clamp device can include only the first member and the second member. In additional arrangements, the clamp device can further include a third member and a fourth member connected together in an end to end arrangement and defining an opening in the clamp device that is sized and configured to pass over an outside surface of the LAA. In any additional arrangements disclosed herein, the device can further include a delivery catheter having an outer sheath and a guide lumen, the guide lumen configured to receive and track over the second guide device. Additionally, the first member of the clamp device can be rigid and the second member of the clamp device can comprise a suture.

Disclosed herein are additional arrangements of methods of closing or occluding an LAA. In any arrangements disclosed herein, the method can include advancing a first guide device into the LAA, advancing a second guide device into a pericardial space outside of the LAA, approximately aligning an end portion of the second guide device with an end portion of the first guide device, advancing a delivery catheter over the second guide device, advancing a clamp device having a first member and a second member from the delivery catheter, opening the clamp device from a closed position to an open position, advancing the clamp device over an outside surface of the LAA toward a neck portion of the LAA, and/or substantially flattening and closing the neck portion of the LAA by closing the clamp device from the open position to the closed position.

Any arrangements of the methods of closing or occluding the LAA can include, in additional arrangements, one or more of the following features, components, steps, and/or details, in any combination with any of the other features, components, steps, and/or details of any other arrangements disclosed herein: wherein moving the clamp device from the closed position to the open position comprises advancing the clamp device past a distal end of the delivery catheter so that the clamp device can automatically move to the open position; wherein the delivery catheter has a guide lumen, the guide lumen being configured to receive and track over the second guide device; wherein the delivery catheter has an outer sheath; wherein at least one of the first and second members of the clamp device is substantially rigid; wherein at least one of the first and second members of the clamp device has a substantially planar contact surface, the contact surface being the surface configured to contact an outside surface of the LAA; wherein the delivery catheter has an outer sheath; wherein the clamp device comprises a least four substantially rigid members connected together in an end to end arrangement and defining an opening in the clamp device that is sized and configured to pass over an outside surface of the LAA; and/or wherein the clamp device comprises at least one rigid member and at least one flexible member interconnected with the at least one rigid member.

Additionally, any implant and/or device or system arrangements disclosed herein can be adapted and/or used for treatment of any tissue condition in a body that is desired to be occluded, restricted, or closed. For example, and without limitation, some arrangements of the devices and systems for treating a tissue condition disclosed herein can include an implant comprising a contact member that can be (but is not required to be) configured to move between a first state and a second state and a securing element, wherein the contact member can be configured to move from the first state to the second state so that at least a portion of the contact member engages a wall portion of the tissue condition after the contact member has been advanced into the tissue condition, the contact member can be configured to rotate at least in a first direction from a first rotational position to a second rotational position, the contact member can be configured to twist at least a portion of the tissue of the tissue condition in the first direction when the contact member is rotated from the first rotational position to the second rotational position, and/or the securing element can be configured to prevent a rotation of at least a portion of the tissue of the tissue condition in a second direction when the securing element is in an operable state, wherein the second direction is opposite to the first direction. In any arrangements, the tissue condition can be a cavity, a chamber, an opening, a passageway, a tear in the tissue, two adjacent or adjoining tissue surfaces, or otherwise.

Further, some arrangements of the devices and systems for treating a tissue condition disclosed herein can include an implant having a contact member that can be (but is not required to be) configured to move between a first state and a second state, a catheter configured to advance the contact member into the tissue condition when the contact member is in the first state and to cause the contact member to move from the first state to the second state so that an outside surface of the contact member engages at least one wall surface of the tissue condition after the contact member has been advanced into or adjacent to the tissue condition, wherein the catheter is configured to exert a torque on the contact member when at least a portion of the catheter is rotated until a predetermine torque level is reached to rotate the contact member from a first rotational position to a second rotational position so that the contact member can twist at least a portion of the tissue condition. In any arrangements, the tissue condition can be a cavity, a chamber, an opening, a passageway, a tear in the tissue, two adjacent or adjoining tissue surfaces, or otherwise.

Further, some arrangements of the devices and systems for treating a tissue condition disclosed herein can include a method of treating a tissue condition, comprising advancing a deployment device having an implant into or adjacent to the tissue condition, wherein the implant can be (but is not required to be) configured to be moved from a first state to a second state, and wherein at least a portion of the implant can be enlarged in a radial direction when the implant is in the second state as compared to the first state, moving the implant from the first state to the second state within the tissue condition so as to move at least a portion of an outside surface of the implant or one or more tissue anchors extending away from an outer surface of the implant against at least one wall surface of the tissue condition, rotating the implant from a first rotational position to a second rotational position to twist the tissue condition, and/or preventing the implant from rotating back to the first rotational position.

Additionally, any implant and/or device or system arrangements disclosed herein can be adapted and/or used for treatment of any tissue condition in a body that is desired to be occluded, reshaped, restricted, or closed. For example, and without limitation, some arrangements of the devices and systems for treating a tissue condition disclosed herein can include an implant comprising a contact member that is configured to engage a wall portion of the tissue condition after the contact member has been advanced into the tissue condition, the contact member can be configured to rotate at least in a first direction from a first rotational position to a second rotational position, the contact member can be configured to twist at least a portion of the tissue of the tissue condition in the first direction when the contact member is rotated from the first rotational position to the second rotational position, and/or the securing element can be configured to prevent a rotation of at least a portion of the tissue of the tissue condition in a second direction when the securing element is in an operable state, wherein the second direction is opposite to the first direction. In any arrangements, the tissue condition can be a cavity, a chamber, an opening, a passageway, a tear in the tissue, two adjacent or adjoining tissue surfaces, or otherwise.

Further, some arrangements of the devices and systems for treating a tissue condition disclosed herein can include an implant having a contact member, a catheter configured to advance the contact member into the tissue condition so that the contact member engages at least one wall surface of the tissue condition after the contact member has been advanced into or adjacent to the tissue condition, wherein the catheter is configured to exert a torque on the contact member when at least a portion of the catheter is rotated until a predetermine torque level is reached to rotate the contact member from a first rotational position to a second rotational position so that the contact member can twist at least a portion of the tissue condition. In any arrangements, the tissue condition can be a cavity, a chamber, an opening, a passageway, a tear in the tissue, two adjacent or adjoining tissue surfaces, or otherwise.

Further, some arrangements of the devices and systems for treating a tissue condition disclosed herein can include a method of treating a tissue condition, comprising advancing a deployment device having an implant into or adjacent to the tissue condition, and wherein at least a portion of the implant engages a wall surface of the tissue condition, rotating the implant from a first rotational position to a second rotational position to twist the tissue condition, and/or preventing the implant from rotating back to the first rotational position.

Disclosed herein are arrangements of a device for treating a left atrial appendage that can include an implant that can have a contact member configured to engage an inside tissue surface of the left atrial appendage and configured to rotate in at least a first direction from a first position to at least a second position so as to twist the left atrial appendage when the contact member is engaged with an inside tissue surface of the left atrial appendage, and a securing element configured to move between a first position in which the securing element is decoupled from the contact member and a second position in which the securing element is coupled with the contact member. In some arrangements, the contact member can be configured to rotate at least in the first direction from the first position to the second position when a torque is applied to the contact member.

Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: wherein the contact member can be configured to rotate at least in the first direction from a first position to at least a second position to twist the left atrial appendage and reduce a size of an ostium of the left atrial appendage from a first size to a second size when the contact member is engaged with an inside tissue surface or the left atrial appendage; wherein the implant can be configured to inhibit the ostium of the left atrial appendage from enlarging back to the first size; wherein the device can be configured such that the contact member can be removed from the left atrial appendage after the securing element has been deployed to the operable state of the securing element; wherein the device can be configured such that the contact member can be removed from the left atrial appendage after the securing element has been deployed to the operable state of the securing element, and wherein the securing element can be configured to prevent a rotation of the tissue of the left atrium and/or the left atrial appendage that has been constricted as a result of the rotation of the contact member from the first position to the second position; wherein the contact member can be configured to move between a first state and a second state, wherein an outside dimension of the contact member can be greater in the second state than in the first state; wherein the contact member can be biased to remain in the second state after deployment into the left atrial appendage; wherein the contact member can be configured to have an approximately fixed and unchangeable size and shape; wherein the contact member can be self-expandable such that the contact member will automatically expand from the first state to the second state when a restraint is removed from the implant without further intervention from a user; wherein the contact member can be configured to automatically move from the first state to the second state when a restraint is removed from the contact member, and wherein the contact member can be configured to engage a wall portion of the left atrial appendage when the contact member is in the second state and advanced into the left atrial appendage; wherein the contact member can have a plurality of tissue anchors on an outer surface thereof; and/or wherein the plurality of tissue anchors on or adjacent to the outer surface of the contact member are configured to engage an inner wall surface of the left atrial appendage after the contact member has been moved to the second state.

Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: wherein the device can be configured to cause a tissue of the left atrium and/or the left atrial appendage to constrict around an outer surface of a body portion of the implant when the contact member is rotated to the second position; wherein the securing element can be configured to engage with the tissue that has constricted around the outer surface of the body portion of the implant to prevent rotation of the implant in a second direction that is opposite to the first direction; wherein, in an operable position, the securing element can be configured to at least inhibit the contact member from rotating back to the first position; wherein the securing element can be configured to prevent a rotation of at least a portion of the left atrial appendage in a second direction when the securing element is implanted in a tissue surface surrounding an ostium of the left atrial appendage, wherein the second direction is opposite to the first direction; wherein the securing element can be configured to at least expand from a first state to a second state, wherein an outside dimension of the securing element can be greater in the second state than in the first state; wherein the securing element can include a plurality of arms; wherein the securing element can have a plurality of struts and a plurality of interconnections between adjacent struts of the plurality of struts; wherein at least an end portion of each of the plurality of arms of the securing element point generally away from the contact member when the securing element is in the first state and point generally toward the contact member when the securing element is in the second state; including a restraint configured to be movable in an axial direction relative to at least a portion of the securing element from a first position in which the plurality of arms of the securing element are restrained by the restraint to a second position in which the plurality of arms of the securing element are not restrained by the restraint;

Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: including a restraint configured to be movable in an axial direction relative to at least a portion of the securing element between a first position in which the plurality of arms of the securing element are restrained by the restraint and a second position in which the plurality of arms of the securing element are not restrained by the restraint, wherein the second axial position can be closer to the first portion of the implant than the first axial position; wherein the restraint can be configured to be movable in an axial direction relative to at least a portion of the securing element from the second position in which the plurality of arms of the securing element are not restrained by the restraint to the first position in which the plurality of arms of the securing element are restrained by the restraint to facilitate repositioning and/or removal of the implant; including a threaded member configured such that rotating the threaded member will cause the restraint to move from the first position to the second position; wherein the restraint can be rotatable relative to the threaded member so that the restraint is not forced to rotate as the threaded member is rotated; wherein the securing element can have a helical shape and can be configured to rotate about a body portion of the implant during the implantation procedure; wherein only a portion of the securing element extends into the left atrium after deployment of the device, and all other portions of the device are internal to the left atrial appendage after deployment of the device; wherein the securing element can be movable between a first state in which the securing element can spin freely relative to the contact member and a second state in which the securing element can be rotationally locked to the contact member; wherein one of the securing element and the contact member can have recesses and the other of the securing element and the contact member can have protrusions configured to selectively engage with the recesses such that the protrusions are spaced apart from the recesses when the securing element is in the first state and the protrusions are engaged with the recesses when the securing element is in the second state; further including a retention element configured to selectively couple the securing element to the contact member at any of a range of selectable distances when the securing element is in the second position; wherein the retention element can have a threaded shaft configured to threadedly engage with the contact member, the threaded shaft being coupled with the securing element; wherein the retention element can be adjustable so as to move the securing element between at least a first position and a second position, wherein the securing element can be closer to the contact member when the retention element is in the second position as compared to when the retention element is in the first position; wherein the retention element can have a threaded member, wherein a rotation of the threaded member in a first direction causes the securing element to move toward the contact member and a rotation of the threaded member in a second direction causes the securing element to move away from the contact member; wherein the retention element can be configured to slide at least in an axial direction over an inner core component of a delivery catheter; wherein the second position can be at least one-quarter of a complete rotation relative to the first position; wherein the second position can be at least one-half of a complete rotation relative to the first position; wherein the second position can be from approximately one-quarter of a complete rotation to one or more complete rotations relative to the first position; including a catheter selectively coupled with the contact member and configured to exert a torque on the contact member to rotate the contact member from the first position until a threshold predetermined torque level is reached; wherein a threshold predetermined torque level can be from approximately 0.25 in-oz of torque to approximately 10 in-oz of torque; and/or wherein a threshold predetermined torque level can be from approximately 0.5 in-oz of torque to approximately 5 in-oz of torque; and/or wherein only approximately 10% or less of an overall length of the deployed device extends into the left atrium after deployment of the device.

Also disclosed herein are arrangements of a method of treating a left atrial appendage that can include advancing a deployment device having an implant into the left atrium, moving at least a portion of an outer surface of a first portion of the implant and/or one or more tissue anchors on or adjacent to the outer surface of the first portion of the implant against an inner wall surface of the left atrial appendage, and rotating the first portion of the implant from a first position to a second position to twist the left atrial appendage from a first position to a second position, and moving a second portion of the implant from a first state in which the second portion of the implant spins freely relative to the first portion of the implant to a second state in which the second portion of the implant can be rotationally locked to the first portion of the implant. Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: wherein the second portion of the implant can be spaced apart from the first portion of the implant when the second portion of the implant is in the first state and the second portion of the implant is engaged with the first portion of the implant when the second portion of the implant is in the second state; including rotating the first portion of the implant until at least a portion of the left atrial appendage constricts around a portion of the implant; including rotating the first portion of the implant until an ostium of the left atrial appendage constricts around a portion of the implant; wherein the method can have engaging with the second portion of the implant a tissue that has constricted as a result of the rotation of the first portion of the implant; and/or wherein moving the second portion of the implant from the first state to the second state causes the second portion of the implant to inhibit a rotation of the left atrial appendage toward the first position of the left atrial appendage.

Also disclosed herein are arrangements of a device for treating a left atrial appendage that can include an implant device that can include a first implant member configured to engage an inside tissue surface of a first portion of the left atrial appendage and a second implant member configured to engage an inside tissue surface of a second portion of the left atrial appendage spaced apart from the first portion of the left atrial appendage. In some arrangements, the device can be configured to rotate the first implant member in a first direction. In some arrangements, the device can be configured to rotate the second implant member in a second direction that is opposite to the first direction.

Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: further including a first core member coupled with the first implant member and configured to cause a rotation of the first implant member when the first core member is rotated; wherein the first implant member can be selectively removably coupled with the first core member so that the first implant member can remain in the left atrial appendage after the first core member has been withdrawn; further including a second core member coupled with the second implant member and configured to cause a rotation of the second implant member when the second core member is rotated; wherein the second implant member can be selectively removably coupled with the second core member so that the second implant member can remain in the left atrial appendage after the second core member has been withdrawn; and/or further including a securing element configured to inhibit a rotation of the first implant member and/or the second implant member in an operable state.

Disclosed herein are arrangements of a device for treating a left atrial appendage that can include an implant device that can include a first implant member configured to engage an inside tissue surface of a first portion of the left atrial appendage and a second implant member configured to engage an inside tissue surface of a second portion of the left atrial appendage spaced apart from the first portion of the left atrial appendage. In some arrangements, the device can be configured to rotate the first implant member in a first direction. Further, in some arrangements, the device can be configured to also rotate the second implant member in the first direction. Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: further including a first core member coupled with the first implant member and configured to cause a rotation of the first implant member when the first core member is rotated; wherein the first implant member can be selectively removably coupled with the first core member so that the first implant member can remain in the left atrial appendage after the first core member has been withdrawn; further including a second core member coupled with the second implant member and configured to cause a rotation of the second implant member when the second core member is rotated; wherein the second implant member can be selectively removably coupled with the second core member so that the second implant member can remain in the left atrial appendage after the second core member has been withdrawn; and/or further including a securing element configured to inhibit a rotation of the first implant member and/or the second implant member in an operable state.

Disclosed herein are arrangements of a device for treating a left atrial appendage that can include an implant that can include a contact member and a securing element coupled with or coupleable with the contact member, the securing element including a plurality of struts and a plurality of interconnections between adjacent struts of the plurality of struts. In some arrangements, the contact member can be configured to rotate at least in a first direction from a first rotational position to a second rotational position to twist at least a portion of the left atrial appendage in the first direction when the contact member is rotated from the first rotational position to the second rotational position. Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: wherein the contact member can be configured to configured to move at least from a first state to a second state so that at least a portion of the contact member can expand radially to engage a wall portion inside the left atrial appendage; wherein the plurality of interconnections provide a point of connection between the adjacent struts of the plurality of struts; wherein the plurality of struts can have a plurality of pairs of struts, wherein each of the pairs of struts can have two struts that are interconnected at a distal end portion of the struts; wherein the plurality of struts can have a first strut, a second strut, and a third strut, the second strut can be positioned between the first strut and the third strut and the second strut can be interconnected with the first strut at a distal end of the first and second struts; wherein the second strut can be interconnected with the third strut at a middle portion of the second and third struts; wherein the implant further can have a retention element coupled with the securing element, the retention element configured to move the securing element in an axial direction toward or away from the contact member or to hold the securing element in a stationary position relative to the contact member; wherein the securing element can be configured to prevent a rotation of at least a portion of the left atrial appendage in a second direction when the securing element is in an operable state, wherein the second direction is opposite to the first direction; wherein the contact member can be self-expandable such that at least a portion of the contact member automatically expands from the first state to the second state when a restraint is removed from the contact member; wherein the implant can be substantially collapsed when the implant is in the first state and is expanded when the implant is in the second state such that a size of the implant can be bigger when the implant is in the second state than when the implant is in the first state; wherein the contact member can be biased to remain in the second state after deployment into the left atrial appendage; wherein the contact member can be configured to be rotated in a clockwise or a counter-clockwise direction; wherein the device can be configured to cause a tissue of the left atrium and/or the left atrial appendage to constrict around an outer surface of a body portion of the implant when the contact member is rotated to the second rotational position, and the securing element is configured to engage with the tissue that has constricted around the outer surface of the body portion of the implant to prevent rotation of the implant in the second direction; wherein the securing element can have a plurality of tissue anchors configured to engage with an internal wall of the heart adjacent to the left atrial appendage; wherein the securing element can have a helical shape and can be configured to rotate about a body portion of the implant during the implantation procedures; wherein the implant can be configured to rotate in a first direction from the first rotational position to the second rotational position; and/or wherein the implant can be configured to prevent rotation of the implant in a second direction after the implant has been fully deployed, wherein the second direction is opposite to the first direction.

Any arrangements of the methods, devices and systems for treating a left atrial appendage disclosed herein can include, in additional arrangements, one or more of the following steps, features, components, and/or details, in any combination with any of the other steps, features, components, and/or details of any other arrangements disclosed herein: wherein the contact member can have a plurality of tissue anchors on an outside surface thereof; wherein the plurality of tissue anchors on the outside surface of the contact member are configured to engage an inner wall surface of the left atrial appendage after the contact member has been moved to the second state; wherein the tissue anchors of the contact member have a proximal facing surface that is angled toward a proximal end of the contact member by 5°; wherein the tissue anchors wherein the tissue anchors of the contact member have a proximal facing surface that is angled toward a proximal end of the contact member at an angle from 2° to 10°; wherein the implant can have a securing element configured to engage with a tissue portion of the heart adjacent to the left atrial appendage; wherein the second rotational position can be at least one-quarter of a complete rotation relative to the first rotational position; wherein the second rotational position can be at least one-half of a complete rotation relative to the first rotational position; wherein the second rotational position can be from approximately one-quarter of a complete rotation to one or more complete rotations relative to the first rotational position; including a catheter selectively coupled with the contact member and configured to exert a torque on the contact member to rotate the contact member from the first rotational position until a threshold predetermined torque level is reached; wherein a threshold predetermined torque level can be from approximately 0.25 in-oz of torque to approximately 10 in-oz of torque; wherein a threshold predetermined torque level can be from approximately 0.5 in-oz of torque to approximately 5 in-oz of torque; including a retention element configured to bias the securing element toward a tissue wall of the LAA; including a retention element configured to bias the securing element toward the contact member; including a retention element configured to couple the securing element with the contact member; wherein the retention element can have a threaded shaft; wherein the device can be configured such that a rotation of the retention element in a first direction causes the securing element to move toward the contact member; wherein the contact member can be configured to rotate at least in a first direction from a first rotational position to a second rotational position when a torque is applied to the contact member; wherein the device can be configured such that the contact member can be removed from the left atrial appendage after the securing element has been deployed to the operable state of the securing element; wherein the device can be configured such that the contact member can be removed from the left atrial appendage after the securing element has been deployed to the operable state of the securing element, and wherein the securing element can be configured to prevent a rotation of the tissue of the left atrium and/or the left atrial appendage that has been constricted as a result of the rotation of the contact member from the first rotational position to the second rotational position; wherein only a portion of the securing element extends into the left atrium after deployment of the device, and all other portions of the device are internal to the left atrial appendage after deployment of the device; wherein only approximately 10% or less of an overall length of the deployed device extends into the left atrium after deployment of the device; wherein the device can be configured for use by a surgical robot device or system; wherein the contact member and the securing element are integrally and/or monolithically formed; and/or wherein the device can be configured to cause a tissue of the left atrium and/or the left atrial appendage to constrict around an outer surface of a body portion of the implant when the contact member is rotated to the second rotational position, and the securing element can be configured to compress the tissue that has constricted around the outer surface of the body portion of the implant between a distal surface of the securing element and the contact member to prevent rotation of the implant in the second direction.

Some arrangements enclosed herein include a surgical robotic device that can include one or more robotic arms and the device of any of the arrangements disclosed herein, wherein the device can be configured for use by the surgical robotic device.

Some arrangements of methods of treating a left atrial appendage disclosed herein can include rotating the left atrial appendage and securing the left atrial appendage in a rotated position. Any arrangements of the methods of treating, closing, or occluding the LAA can include, in additional arrangements, one or more of the following features, components, steps, and/or details, in any combination with any of the other features, components, steps, and/or details of any other arrangements disclosed herein: wherein rotating the left atrial appendage comprises rotating the left atrial appendage to deform or occlude the left atrial appendage; wherein securing the left atrial appendage in a rotated position comprises securing the left atrial appendage in a rotated position in which the left atrial appendage is reduced in volume; wherein securing the left atrial appendage in a rotated position comprises securing the left atrial appendage in a rotated position in which the left atrial appendage is deformed or occluded; wherein rotating the left atrial appendage comprises bending or contorting the left atrial appendage; wherein securing the left atrial appendage in a rotated position comprises securing the left atrial appendage in a position in which a blood communication between the left atrial appendage and a left atrium is inhibited, eliminated, or substantially eliminated; wherein rotating the left atrial appendage comprises engaging a wall portion on an inside of the left atrial appendage and/or an ostium of the left atrial appendage with a contact member and rotating the contact member; wherein the contact member is positioned on an implant coupled to the delivery system; wherein the contact member is self-expanding, balloon expandable, mechanically expanded, and/or a balloon; wherein rotating the left atrial appendage comprises engaging a wall portion on an inside of the left atrial appendage with one or more tissue anchors, one or more tissue grippers, and/or one or more other tissue holding features; wherein rotating the left atrial appendage comprises advancing a device into the left atrial appendage and rotating at least a component of the device to rotate the left atrial appendage; wherein rotating at least a component of the device comprises rotating at least the component of the device from approximately 90° to approximately 360° in either direction from an initial position; wherein rotating the left atrial appendage comprises rotating a portion of the left atrial appendage about an axis to twist the left atrial appendage; wherein rotating a portion of the left atrial appendage about one or more axes from an initial position comprises rotating the portion of the left atrial appendage from approximately 90° to approximately 360° in either direction from the initial position; wherein rotating a portion of the left atrial appendage comprises rotating the left atrial appendage until an opening of the left atrial appendage is substantially or completely closed; wherein rotating a portion of the left atrial appendage comprises rotating the left atrial appendage until a blood communication between the left atrial appendage and a left atrium is inhibited; wherein rotating a portion of the left atrial appendage comprises rotating the left atrial appendage until a communication of blood or other matter between the left atrial appendage and the left atrium is eliminated or substantially eliminated; wherein securing the left atrial appendage in a rotated position comprises engaging tissue of the heart that has been twisted; wherein engaging tissue of the heart that has been twisted comprises engaging tissue wall with an anchor element or gripping element; wherein securing the left atrial appendage in a rotated position comprises securing a tissue of the heart outside of an occluded portion of the left atrial appendage with an anchor element; wherein securing the left atrial appendage in a rotated position comprises securing a tissue of an occluded portion of the left atrial appendage with an anchor element; and/or wherein the anchor element comprises a plurality of tissue grippers on at least one surface thereof configured to engage with the internal wall of the heart outside of the left atrial appendage.

Some arrangements of the method of reducing an ostium of a left atrial appendage disclosed herein can include twisting tissue of the heart to constrict the ostium of the left atrial appendage and securing tissue that has deformed or constricted as a result of twisting tissue of the heart. Any arrangements of the methods of treating, closing, or occluding the LAA can include, in additional arrangements, one or more of the following features, components, steps, and/or details, in any combination with any of the other features, components, steps, and/or details of any other arrangements disclosed herein: wherein securing the tissue that has deformed or constricted comprises advancing a securing element into the tissue that has deformed or constricted as a result of twisting tissue of the heart; wherein the securing element comprises a tissue anchor or tissue gripper; wherein securing the tissue that has deformed or constricted as a result of twisting tissue of the heart comprises advancing a securing element into the tissue that has deformed or constricted to compress the tissue that has deformed or constricted; and/or wherein securing tissue that has deformed or constricted as a result of twisting tissue of the heart comprises advancing one or more sutures or one or more staples into the tissue that has deformed or constricted as a result of twisting tissue of the heart.

Some arrangements of the method of treating a left atrial appendage disclosed herein can include twisting the left atrial appendage such that the left atrial appendage becomes reduced in volume and securing the left atrial appendage in a reduced volume configuration. In some arrangements, securing the left atrial appendage in a reduced volume configuration can include occluding the left atrial appendage with an implant that is smaller in size than a size of the inside of the left atrial appendage. In some arrangements, the method of treating the left atrial appendage can include unsecuring and untwisting the left atrial appendage.

### BRIEF DESCRIPTION OF THE DRAWINGS

FIG. 1A illustrates a path through the venous system via femoral vein and a transseptal puncture into the left atrium that can be used to access the left atrial appendage (LAA).

FIG. 1B shows a section view of a left atrium, showing a guidewire advancing toward the LAA.

FIG. 1C shows a surgeon's left view of the left atrium.

FIG. 1D shows a surgeon's left view of the left atrium, showing a delivery device advancing toward the LAA.

FIG. 1E shows an example of a device being advanced toward the heart of a patient through an access point in the internal jugular vein.

FIG. 2A shows an arrangement of treatment device having an implant device being advanced through a catheter into the LAA, the implant device being in a collapsed state and restrained within an outer tube of the catheter.

FIG. 2B shows the arrangement of the treatment device of FIG. 2A, showing the contact member being expanded within the LAA.

FIG. 2C shows the arrangement of the treatment device of FIG. 2A, showing the contact member being rotated to twist the LAA and cause a neck or opening of the LAA to constrict around a portion of the implant device.

FIG. 2D shows the arrangement of the treatment device of FIG. 2A, showing the securing element of the arrangement of the implant device being advanced toward the contact member of the implant device.

FIG. 2E shows the securing element of the treatment device of FIG. 2A engaged with the patient's tissue that has constricted as a result of the twisting of the LAA.

FIG. 2F shows the implant device of FIG. 2A disengaged and removed from the catheter.

FIG. 2G shows the arrangement of treatment device of FIG. 2A advanced to the left atrium, the implant device being in a collapsed state and restrained within an outer tube of the catheter.

FIG. 2H shows the arrangement of the treatment device of FIG. 2A, showing the contact member being expanded within the left atrium before being advanced into the LAA.

FIG. 2I shows the arrangement of the treatment device of FIG. 2A, showing the contact member being advanced into the LAA after the contact member has been expanded.

FIG. 2J shows the arrangement of the treatment device of FIG. 2A, showing the contact member being rotated to twist the LAA and cause a neck or opening of the LAA to constrict around a portion of the implant device.

FIG. 2K shows another arrangement of treatment device having an implant device being advanced through a catheter into the LAA, the implant device being in a collapsed state and restrained within an outer tube of the catheter.

FIG. 2L shows the arrangement of the implant device of FIG. 2K engaged with the patient's tissue that has constricted as a result of the twisting of the LAA.

FIG. 3A is an exploded perspective cross-sectional view of another example of an implant device.

FIG. 3B is a side elevation view of the implant device of FIG. 3A in a first state.

FIG. 3C is a side elevation view of the implant device of FIG. 3B in a second state.

FIG. 4A is a perspective view of another example of an implant device.

FIG. 4B is a side elevation view of the implant device of FIG. 4A.

FIG. 4C is an end view of the implant device of FIG. 4A.

FIG. 4D is another perspective view of the implant device of FIG. 4A.

FIG. 4E is a perspective cross-sectional view of the implant device of FIG. 4A in a first state.

FIG. 4F is a perspective cross-sectional view of the implant device of FIG. 4A in a second state.

FIG. 4G is an exploded perspective cross-sectional view of the implant device of FIG. 4A.

FIG. 4H is an exploded perspective view of the implant device of FIG. 4A.

FIG. 4I is a side elevation view of the implant device of FIG. 4A in the first state.

FIG. 4J is a side elevation view of the implant device of FIG. 4A in the second state.

FIG. 5 is a perspective view of a first variation of the implant device of FIG. 4A.

FIG. 6 is a perspective view of a second variation of the implant device of FIG. 4A.

FIG. 7 shows an experimental setup for demonstrating an arrangement of the implant device of FIG. 4A.

FIG. 8A shows an arrangement of a system for closing or occluding an LAA including a delivery system and the implant device of FIG. 4A.

FIG. 8B shows a portion of the arrangement of a system for closing or occluding an LAA shown in FIG. 8B.

FIG. 9A shows a first isometric view of a portion of an arrangement of a delivery catheter that can be used with any arrangements of the implant disclosed herein.

FIG. 9B shows a second isometric view of the arrangement of the delivery catheter shown in FIG. 9A.

FIG. 9C shows a first side view of the arrangement of the delivery catheter shown in FIG. 9A.

FIG. 9D shows a second side view of the arrangement of the delivery catheter shown in FIG. 9A.

FIG. 9E shows a top view of the arrangement of the delivery catheter shown in FIG. 9A.

FIG. 9F shows a bottom view of the arrangement of the delivery catheter shown in FIG. 9A.

FIG. 9G shows a front view of the arrangement of the delivery catheter shown in FIG. 9A.

FIG. 9H shows a back view of the arrangement of the delivery catheter shown in FIG. 9A.

FIG. 10A shows a side view of the arrangement of the implant device of FIG. 4A and distal end portion of the delivery catheter shown in FIG. 10A.

FIG. 10B shows a top view of the arrangement of the implant device of FIG. 4A and distal end portion of the delivery catheter shown in FIG. 10A.

FIG. 10C shows an isometric view of the arrangement of the implant device of FIG. 4A and distal end portion of the delivery catheter shown in FIG. 10A.

FIG. 11A shows a side view of an arrangement of a distal end portion of the outer sheath, wherein the implant device of FIG. 4A is positioned within the outer sheath of the delivery catheter.

FIG. 11B shows a cutaway view of a portion of the arrangement of the distal end portion of the outer sheath shown in FIG. 11A, wherein the implant device of FIG. 4A is positioned within the outer sheath of the delivery catheter.

FIG. 11C shows a side view of the arrangement of the distal end portion of the outer sheath shown in FIG. 11A, wherein the implant device of FIG. 4A is positioned partially past the distal end of the outer sheath such that the securing element is positioned within the outer sheath and the contact member is positioned past the distal end of the outer sheath.

FIG. 11D shows a cutaway view of the arrangement of the distal end portion of the outer sheath shown in FIG. 11A, wherein the implant device of FIG. 4A is positioned partially past the distal end of the outer sheath such that the securing element is positioned within the outer sheath and the contact member is positioned past the distal end of the outer sheath.

FIG. 11E shows a side view of the arrangement of the distal end portion of the outer sheath shown in FIG. 11A, wherein the implant device of FIG. 4A is positioned past the distal end of the outer sheath and the contact member and the securing element are in the expanded state, but are spaced apart.

FIG. 11F shows a side view of the arrangement of the distal end portion of the outer sheath shown in FIG. 11A, wherein the implant device of FIG. 4A is positioned past the distal end of the outer sheath, the contact member and the securing element are in the expanded state and are positioned close together.

FIGS. 12A-12D show a side view of the arrangement of the delivery catheter shown in FIG. 12A and a partial cutaway view of the distal end portion of the outer sheath and the implant device of FIG. 4A in various stages of deployment of the implant.

FIGS. 13A-13E show a side view of the arrangement of the delivery system shown in FIG. 13A and a side view of the outer sheath and the implant device of FIG. 4A in various stages of deployment of the implant.

FIG. 14 shows a flow chart representing an example of a method of using the delivery system of FIG. 13A.

FIG. 15 shows a flow chart representing an example of a set of steps that may be used to deliver the implant device of FIG. 4A, as part of the method of FIG. 14.

FIG. 16A is a schematic side view of a distal portion of the delivery system of FIG. 13A, with a securing element of the implant device of FIG. 4A in a first longitudinal position during a recapture process.

FIG. 16B is a schematic side view of a distal portion of the delivery system of FIG. 13A, with the securing element of the implant device of FIG. 4A in a second longitudinal position during a recapture process.

FIG. 16C is a schematic side view of a distal portion of the delivery system of FIG. 13A, with the securing element of the implant device of FIG. 4A in a third longitudinal position during a recapture process.

FIG. 16D is a schematic side view of a distal portion of the delivery system of FIG. 13A, with the securing element of the implant device of FIG. 4A in a fourth longitudinal position during a recapture process.

FIG. 17 is a top plan view of the securing element of the implant device of FIG. 4A, with the securing element in a flat configuration.

FIG. 18 is an enlarged top plan view of a distal end portion of a strut of the securing element of FIG. 17.

FIG. 19 is a top plan view of an example of an alternative securing that may be incorporated into the implant device of FIG. 4A, with the securing element in a flat configuration.

FIG. 20 is an enlarged top plan view of a distal end portion of a strut of the securing element of FIG. 17.

### DETAILED DESCRIPTION

### I. Overview

Described herein are novel devices, systems, and methods for closing or occluding a left atrial appendage (LAA). Some arrangements of the novel devices, systems, and methods for closing or occluding an LAA disclosed herein include a percutaneous transcatheter device intended to reduce the risk of thromboembolism from the LAA in patients with non-valvular atrial fibrillation (NVAF) who are at increased risk for stroke and systemic embolism and are recommended for anticoagulation therapy.

Some arrangements comprise a method that includes advancing a delivery system to the LAA, advancing and deploying an expandable element (which can be, in some arrangements, covered with barbs, texture, or other tissue engaging features or, alternatively, can be smooth) and which can have a generally spherical or orb shaped shape into the left atrial appendage, allowing the expandable element to engage distally and/or radially with inner wall surfaces of the LAA, applying a rotation to the inner catheter member connected to the expandable element to twist the LAA to close and/or occlude the LAA at or near the ostium. By occluding the LAA, some arrangements disclosed herein can effectively eliminate or significantly or nearly completely eliminate a communication of blood or other matter between the left atrium and the LAA. Any methods of deployment disclosed herein can also include deployment of a securing element (which is also referred to herein as a locking element or anchoring element) that is configured to inhibit or prevent the unwinding of the expandable element relative to the LAA and the left atrium ostial tissue, thereby inhibiting or preventing the untwisting of the LAA.

The devices, systems, and methods disclosed herein can be used, or can be adapted, for other applications within the body or on the surface of the body of any human, animal, reptile, or other living being. Other applications include, without limitation, closing openings in other tissues aside from the LAA, occluding or closing openings, passageways, and/or chambers within the heart or other organs, occluding or closing holes or other slits or openings in vessels and passageways, and/or treating other conditions.

The clinical benefit of some arrangements is a resultant implant which is not in direct blood contact with the left atrial blood or flow except a possible portion of the securing feature. The securing element of any arrangements can be configured to limit the exposure of the securing element to the blood within the left atrium (i.e., to limit the amount of the securing element that projects into the left atrium). In some arrangements, the entire implant can be surrounded by tissue of the LAA tissue so that no portion, or only a minimal portion (for example, less than 10% of the surface area, or less than 40% of the surface area) of the implant is exposed to blood flow within the left atrium. This can have clinical benefits to the patient as there should be post drug regiment required. Any of the devices used in any of the methods described here may be advanced under any of a variety of visualization techniques, e.g., fluoroscopic visualization, ultrasound, etc.

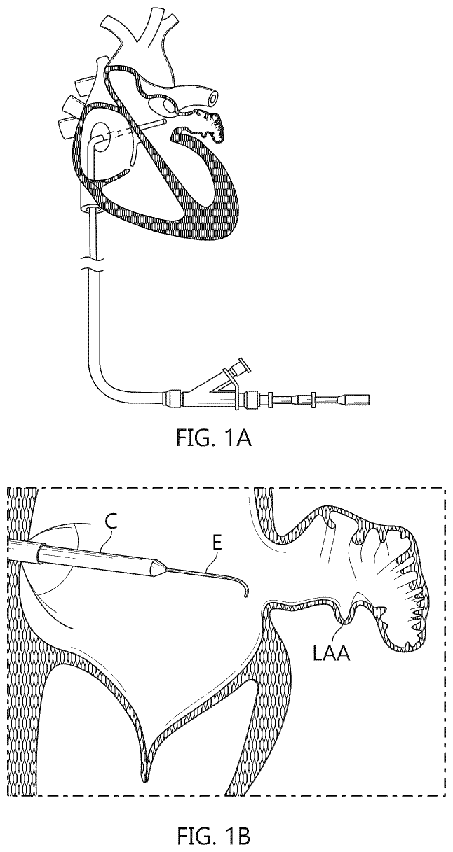

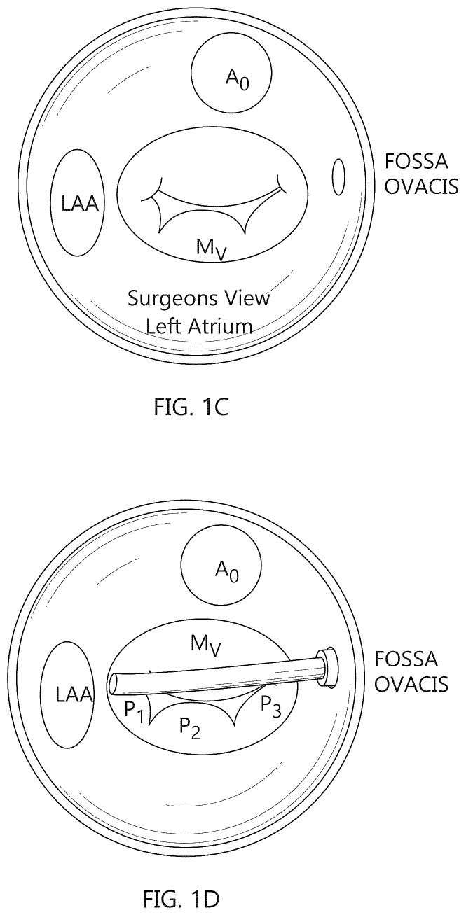

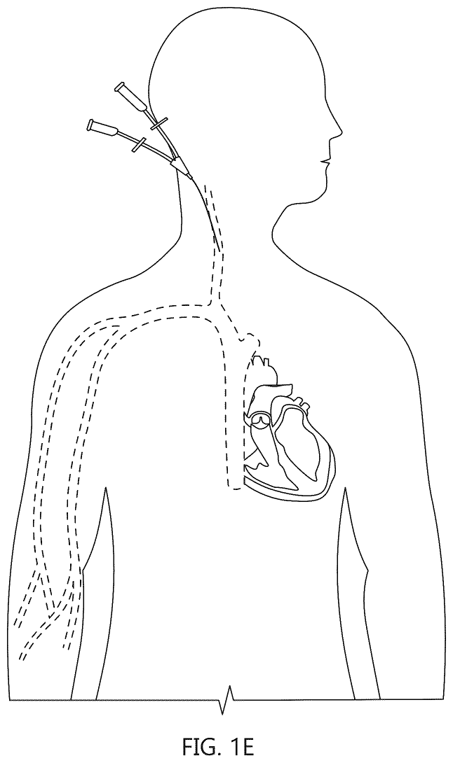

For any of the arrangements disclosed herein, access to the LAA can be gained by any number of suitable means or access points. For example, and without limitation, access to the LAA for some arrangements can be gained by entering through the venous system via femoral vein and a transseptal puncture into the left atrium. Imaging could use both fluoroscopy and echo (TEE, ICE or transthoracic) to image the size, position, and location of the LAA for entry of the prosthesis or device for occlusion. FIGS. 1A-1D show a portion of an example of a path from an access site to the LAA.

Entering through the venous system via femoral vein and a transseptal puncture into the left atrium, access to the left atrial appendage (LAA) for any of the arrangements of the devices, systems, and methods disclosed herein can be gained. Imaging could use both fluoroscopy and echo (TEE, ICE or transthoracic), the size, position, and location of the LAA for entry of the prosthesis for closure. FIGS. 1A-1D show this example of a path from the access site to the LAA. Other access cites for any of the arrangements of the devices, systems, and methods disclosed herein can include access through the internal jugular (IJ) vein, as shown in FIG. 1E.

Further, any device, system, and method arrangements disclosed herein can be delivered to the left atrium/LAA or include delivery to the left atrium/LAA via a transfemoral arterial pathway. In some arrangements, the transfemoral arterial pathway can include advancing the delivery device through the femoral artery, up the aorta, down the aortic valve, up the mitral valve, and into the LAA. Similarly, any device, system, and method arrangements disclosed herein can be delivered to the left atrium/LAA or include delivery to the left atrium/LAA via a transradial pathway, which can include access through a radial artery in the wrist, for example and without limitation. This access pathway is also referred to as transradial access, the transradial approach, or transradial angioplasty.

The implant of any arrangements disclosed herein can have an expandable atraumatic shape with tissue gripping features located on the outer edges of the shape, coupled to a securing and or ratcheting feature which can hold the initial or final closed position of the implant. The implant of any arrangements disclosed herein can be configured to grip the internal tissue of the LAA with radial force as well. In some arrangements a vacuum or suction can be provided by the catheter or any component thereof to draw a tissue portion of the LAA or atrium toward the implant. The implant of any arrangements disclosed herein can have an atraumatic shape that can be spherical, dome shaped, or comprise a coil of wire in the shape of a disk, can have expanded cut pattern in the shape of a stent, or anything else which can have rounded edges. In some arrangements, the barbs (which can be tissue anchors) on the outer edges or surface of the implant can comprise metal hooks, plastic cleats, rough texture of some material or surface features, a coating or activated adhesive which grips the inside surface of the LAA. Additionally, in any arrangements disclosed herein, the tissue anchors can be positioned on or adjacent to an end portion of the implant to engage with an end portion of the LAA. In any arrangements, the barbs can be directional allowing for tissue engagement in one rotational direction and a disengagement in the opposite rotational direction for a possible repositioning, resizing, or removal from the LAA.

The rotation used to twist closed or occluded (completely or substantially) the LAA for any arrangements disclosed herein may be as little as a quarter of a turn (i.e., revolution), a half turn, a complete turn, up to as much as multiple turns for deeper or longer LAAs. The securing feature or element (also referred to herein as an anchoring element) in any arrangements disclosed herein can have a single arm or multiple arms which can be connected to the implant body that is positioned and rotated within the closed or substantially closed LAA. The securing feature or element can also be configured to engage tissue adjacent to the ostium of the LAA. In any arrangements, the securing element can have multiple arms or members, can have an annular ring, can have a disk, or any other suitable shaped surface anchor configured to couple non-twisted tissue to the twisted implant. In some arrangements, the securing element can also have a small diameter ring which can be configured to clamp to or engage with the tissue which contacts to the center hub of the implant (adjacent to the ostium of the LAA) or it can also have a clip which folds and clips the implant to the side of the wall of the left atrium.

In some arrangements disclosed herein, the device can be configured to restrict an opening of the LAA by reducing a cross-sectional area of the opening of the LAA by at least 95%, or by at least 90%, or by from at least approximately 80% to approximately 100% as compared to a cross-sectional area of the opening of the LAA before the device was implanted (including a blockage effect from the device). Further, in some arrangements, the method can include rotating the implant from the first rotational position to the second rotational position to twist the LAA until an ostium of the LAA is at least 95% blocked and/or restricted, or at least 90% blocked and/or restricted, or at least 80% blocked and/or restricted, or from approximately 70% blocked and/or restricted to approximately 100% blocked and/or restricted. Additionally, any arrangements disclosed herein can include implanting two or more implants of any of the implant arrangements disclosed herein in the LAA. For example, and without limitation, any of the implant arrangements disclosed herein can be configured to be deployed or implanted in the LAA to improve the occlusion of implants already implanted in the LAA, including any implants that fit within any of the foregoing ranges of less than complete occlusion. In some arrangements, one or more additional implants or devices can be implanted adjacent to, over, around, or otherwise with an existing implant to improve a level of occlusion of the LAA.

Alternatively, in any arrangements disclosed herein, the securing element can be configured to merely compress the tissue of the left atrium and/or the left atrial appendage that has constricted around an outer surface of a body portion of the implant between a distal surface of the securing element and the contact member to prevent rotation of the implant in the second direction, i.e., after the contact member has been rotated to the second rotational position, without penetrating into such tissue. For example, and without limitation, in any arrangements disclosed herein, the securing element can have a body portion that is smooth an nonobtrusive or nonpenetrating, e.g., so that the securing element does not have any tissue penetrating features on it that extend toward the tissue surfaces. In other arrangements, the arms (or, at least, the portions of the arms that extend in the axial direction when the securing element is in the second state) or other tissue penetrating portions of the securing element can be short, such as from approximately 1 mm to approximately 5 mm in length, or from approximately 1 mm to approximately 3 mm in length, or from approximately 1 mm to approximately 2 mm in length, or of any values or ranges of values between any of the foregoing ranges.

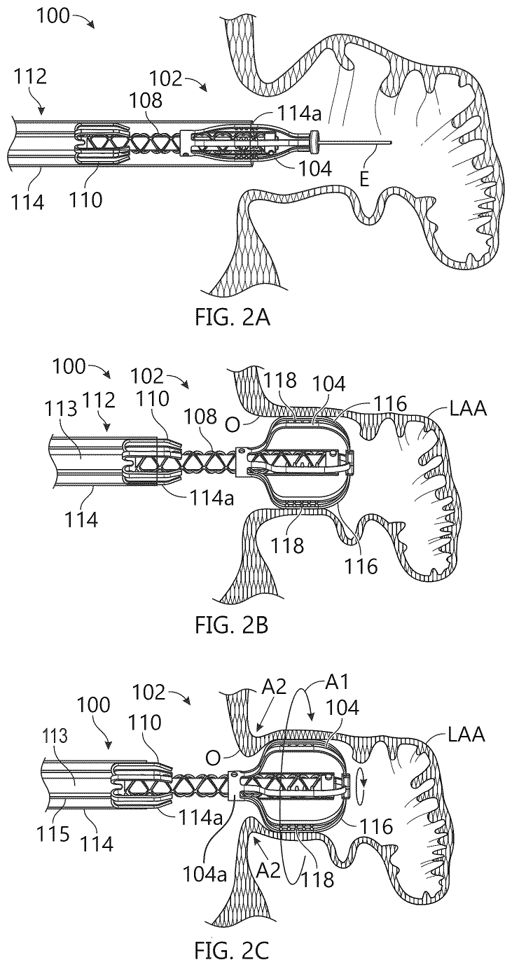

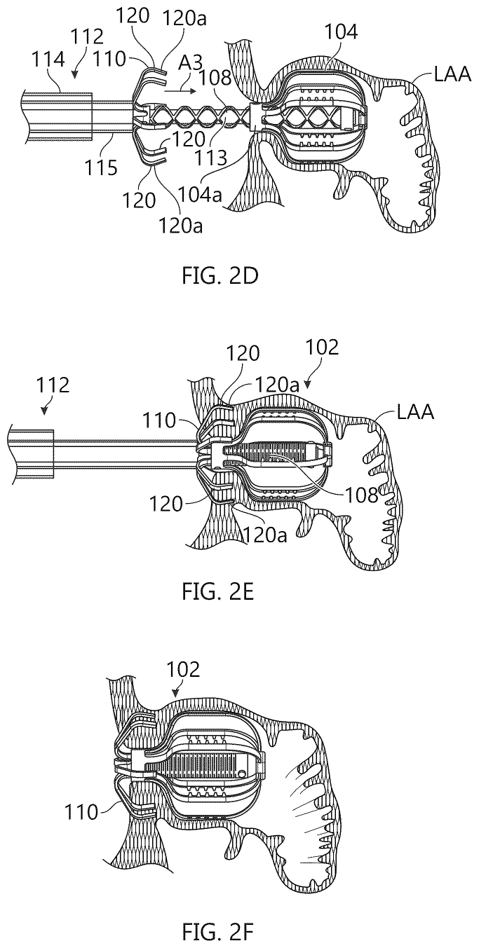

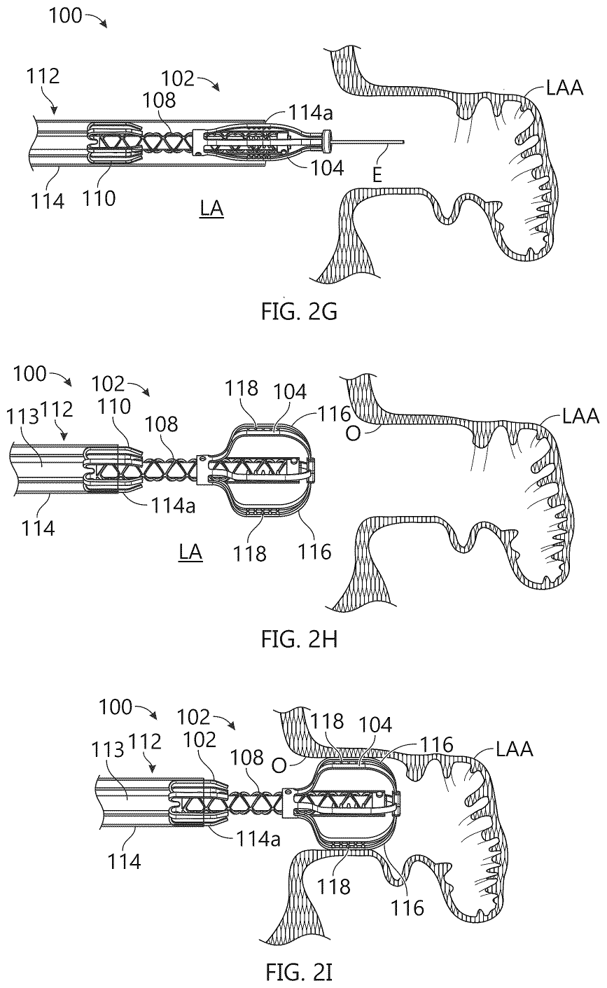

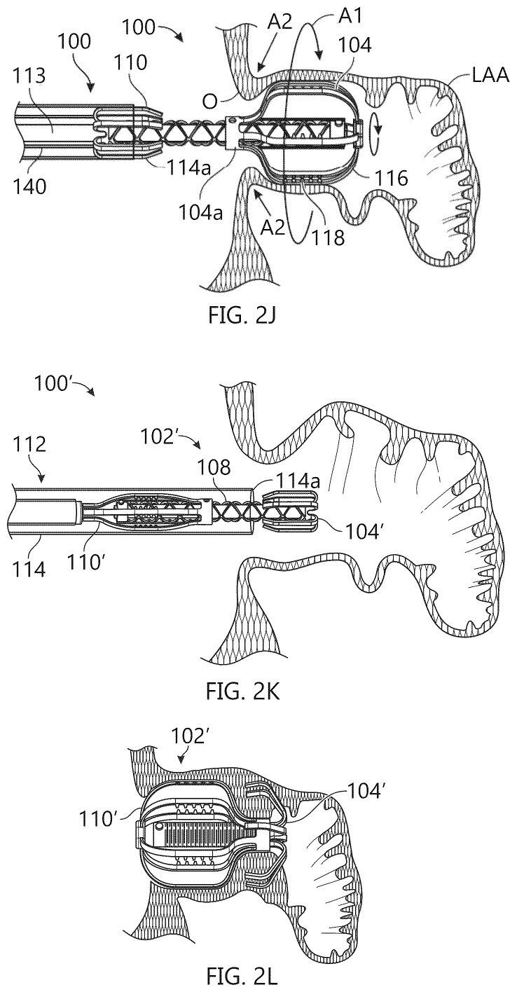

FIGS. 1A and 1B show a section view of a left atrium, showing a guidewire G advancing from a catheter C toward the left atrial appendage LAA. FIG. 2A shows an arrangement of a treatment device 100 for occluding or closing the opening of the LAA (also referred to herein as an occlusion device).

In any arrangements disclosed herein, a rotation of the contact member, implant device, and/or left atrial appendage can comprise rotating the contact member, implant device, and/or left atrial appendage about a longitudinal axis of the contact member and/or implant device. In some arrangements, the axis of rotation can be an axis that extends through the ostium of the LAA towards an internal wall of the LAA, or is an axis that is defined by an insertion path of the implant into the LAA. In some arrangements, the insertion path can be through the ostium of the LAA to a far wall of the LAA. In some arrangements, the axis of rotation can be an axis that extends through the ostium of the LAA towards an internal wall of the LAA and the LAA and/or the implant is rotated about the axis. In some arrangements, the axis of rotation can be an axis that extends through the ostium of the LAA towards an internal wall of the LAA and the LAA and/or the implant is rotated about the axis to twist the LAA.

In any arrangements disclosed herein, the treatment device (including the arrangement of the treatment device 100) can be configured to rotate and twist the LAA so as to cause a neck or a portion of the LAA adjacent to the opening of the LAA to constrict and substantially or fully close about an outside surface of a portion of the implant device, thereby causing the opening of the LAA to be occluded. In any arrangements of the treatment device, including the arrangement of the treatment device 100, the system can have an implant device 102 having a contact member 104 (also referred to in any arrangements disclosed herein as a contact element, a first portion of the implant, or an expandable implant member), a securing member or securing element 110 (also referred to in any arrangements herein as a securing member or a second portion of the implant), and a retention element 108 (also referred to as a retention member). The implant device 102 can be configured to be advanced through a catheter 112 into the LAA. The arrangement of the implant device 102 shown in FIG. 2A is shown in a collapsed state and restrained within an outer sleeve 114 of the catheter 112. As shown, the implant device 102 can be advanced distally out of the catheter 112 past a distal end 114a of the outer sleeve 114 by advancing a portion of or member of the catheter, such as without limitation a core member 113 of the catheter 112, so that the contact member 104 of the implant device 102 can be advanced into the LAA and/or deployed within the LAA.

Alternatively, the catheter 112 having the implant device 102 therein can be advanced into a desired position within the LAA and, while holding the implant device 102 in a stationary axial position by maintaining the core member 113 of the catheter 112 in a stationary axial position, the outer sleeve 114 of the catheter 112 can be retracted or withdrawn so as to expose and/or unrestrain the contact member 104 of the implant device 102. In any arrangements disclosed herein, the contact member 104 can be self-expanding in a radial direction so that, when a restraint is removed from the contact member 104, the contact member 104 can expand against an inner surface or wall of the LAA automatically. In other arrangements, the contact member 104 can be mechanically expandable, such as by a balloon expander, so as to expand against inside surface or wall of the LAA. FIG. 2B illustrates the contact number 104 after it has been expanded against an inside wall of the LAA distal to an ostium or opening O of the LAA.

Alternatively, in any arrangements disclosed herein, the contact member can be configured to remain in a first state within the catheter, during the entire treatment procedure, and/or thereafter. For example, and without limitation, in any arrangements disclosed herein, the contact member can be configured such that the contact member is deployed from the catheter and advanced into contact with a tissue surface of an inside wall of the LAA, engage the tissue surface of the inside wall of the LAA, and cause the LAA to twist when a torque and/or rotation is applied to the contact member, all without changing the state of the contact member. Alternatively, in any arrangements disclosed herein, a contact member can be configured to be advanced into the pericardial space around an outside of the LAA to engage an outside surface of the LAA and to and cause the LAA to twist when a torque and/or rotation is applied to the contact member.

In any arrangements disclosed herein, including the arrangement illustrated in FIG. 2B, the contact member 104 can have a plurality of arms or struts 116 that are each configured to self-expand in a radial direction when a restraint has been removed from an outside surface of the contact member 104. For example without limitation, any arrangements of the contact member disclosed herein can have six struts 116, or between six and ten struts, or from less than six to more than ten struts.

Further, in any arrangements, the contact member 104 can have a plurality of teeth, cleats, barbs, nubs, texture, studs, anchors or other tissue engaging features 118 or other similar features configured to penetrate or engage the tissue of the LAA that are configured to penetrate into a tissue within the LAA when the contact member 104 is expanded against the tissue of the LAA and/or when the contact member 104 is rotated or twisted within the LAA. Note that teeth, cleats, barbs, nubs, texture, studs, anchors and other tissue engaging features or features configured to grip or engage the tissue when torque is applied to the expanded contact member will be collectively referred to herein as tissue anchors, which use of this term is meant to describe and include any of the foregoing features individually and/or any combination of these features.

The tissue anchors 118 can be integrally formed with the struts, on the struts, added to the struts, or otherwise coupled with or supported by the struts. The tissue anchors 118 can be circumferentially facing (as shown, can be radially facing so as to penetrate or engage the tissue at an orthogonal angle relative to the tissue surface of the LAA, at an angle relative to the line that is tangential to the outer surface of the contact member 104, or otherwise. In some arrangements, each strut 116 can support a plurality of tapered tissue anchors facing in a circumferential direction, as illustrated in FIG. 2B. All of the tissue anchors can face in a similar orientation relative to each of the struts, such as in the circumferential direction relative to each strut. In the illustrated arrangement, each strut 104 has five tissue anchors 118. In this arrangement, when the contact member 104 is rotated in a first direction (indicated by arrow A1 in FIG. 2C, which can be in the clockwise or the counterclockwise direction), one or more or all of the struts 116 and one or more or all of the tissue anchors 118 can engage the tissue of the LAA and cause the LAA to twist or rotate in the first direction A1. The twisting or rotation of the LAA in the first direction from a first rotational position to a second rotational position results in the opening or ostium O of the LAA constricting in a radial direction (represented or identified by arrows A2 in FIG. 2C) so that the opening O of the LAA is caused to move or constrict around an outside surface of a proximal portion 104a of the contact member 104. An operator can twist or rotate the contact member 104 by twisting or rotating the core member 113 of the catheter 112. The tightening or constriction of the opening O of the LAA around an outside surface of the proximal portion 104a of the contact member 104 or other portion of the implant device can result in the occlusion, or substantial occlusion, or substantial closing off of the interior portion of the LAA from the remaining chambers within the heart, thereby substantially reducing the health risks associated with an open LAA.

As described in greater detail below, in some instances of use, the physician may rotate contact member 104 through a first range of motion in the first direction (as shown in FIG. 2C), then pull contact member 104 proximally a certain distance, then rotate contact member 104 through a second range of motion in the first direction. This combination of rotational movement and translational movement of contact member 104 may provide a combination of torsional compression and longitudinal compression of the tissue of the LAA. In some such uses, the second range of rotation may be less than the first range of motion. By way of example only, the second range of rotation may be approximately one third of the first range of motion. By way of further example only, contact member 104 may be pulled in the proximal direction along a distance that is approximately equal to the length of contact member between the stage of rotating contact member 104 through the first range of motion and the stage of rotating contact member 104 through the second range of motion.

In some arrangements, as in the illustrated arrangement, the securing element 110 can be maintained in a collapsed or first state such as by being restrained by the outer sleeve 114 of the catheter 112 while the contact member 104 is being deployed and rotated to prevent the securing element 110 from contacting tissue within the heart and potentially lacerating or otherwise damaging such tissue. An intermediary sleeve or tube 115 can be coupled with the securing element 110 and can be used to manipulate and control a position and/or an orientation of the securing element 110, including holding a proximal end portion 110a of the securing element in a fixed axial position while a distally directed force is exerted on the contact member 104 to maintain the retention element in the first, extended state. In any implant device arrangements disclosed herein, the securing element (including, For example, and without limitation, securing element 110) can be keyed, indexed, or otherwise rotationally fixed to the contact member (including, For example, and without limitation, contact member 104) so that the securing element cannot rotate relative to the contact member and the contact member cannot rotate relative to the securing element. In this configuration, the securing element can prevent or substantially prevent or inhibit the contact member and the LAA from rotating back toward the first rotational position.

With reference to FIG. 2D, with the contact member 104 having been rotated to the second rotational position and maintained in the second rotational position such that the opening O of the LAA remains constricted around a proximal portion 104a of the contact member 104 or other portion of the implant device and the LAA is generally occluded from the remainder of the heart chambers, the catheter tube member 115 can then be advanced in a distal direction (represented by arrow A3 as shown in FIG. 2D) or the outer sleeve 114 can be withdrawn in a proximal direction so that the securing element of 110 can be exposed so that it can self-expand from a first, collapsed state (as shown in FIG. 2C) to a second, expanded or open state (as shown in FIG. 2D). In the second state, a plurality of struts or members 120 of the securing element 110 can expand in a generally radial direction so as to open up to a larger overall diameter or profile. Additionally, because each of the one or more members 120 of the securing element 110 can have end portions 120a that extend in a generally distal axial direction (but can be slightly angled inwardly), as the securing element 110 is advanced in the axial direction, the distal portions 120a of each of the one or more members 120 can penetrate into and/or engage with a tissue portion of the heart, as shown in FIG. 2E. The tissue portion that the one or more members 120 can penetrate into or engage with can include portions of the tissue comprising the left atrium and/or portions of the tissue comprising the LAA. As mentioned above, the contact member 104 can be held in generally a stationary axial position using the core member 113 while the securing element 110 is advanced distally toward the contact member 104. The retention element 108 can thereafter be unrestrained so that it can maintain the securing element 110 in the second rotational position wherein the securing element 110 is engaged with the tissue of the heart, as shown in FIG. 2E. In some arrangements, the securing element can be biased toward a smaller size in the axial direction, such as with a spring member or similar. For example, the retention element 108 can be formed by laser cutting openings within a cylindrical tube, such as a hypo tube made of an elastic material, such as Nitinol. Thereafter, with reference to FIG. 2F, the implant device 102 can be disengaged from the catheter 112 and the catheter 112 can be retracted and removed from the patient's body. With the securing element 110 engaged with the patient's tissue, as illustrated in FIG. 2F, the LAA can be prevented from rotating to the first rotational position, which is the untwisted or relaxed position. In this configuration, the implant device 102 can secure and maintain the LAA in a substantially or completely occluded or substantially or completely closed state.

Thereafter, with reference to FIG. 2F, the implant device 102 can be disengaged from the catheter 112 and the catheter 112 can be retracted and removed from the patient's body. With the securing element 110 engaged with the patient's tissue, as illustrated in FIG. 2F, the LAA is prevented or, at least, inhibited or biased from rotating to the first rotational position, which is the untwisted or relaxed position. In this configuration, the implant device 102 can secure and maintain the LAA in a substantially or completely occluded or substantially or completely closed state.

Note that, in any arrangements of the methods and devices disclosed herein, including without limitation any of the methods of treating an LAA, the contact member can be partially or completely expanded in the left atrium (LA) before being advanced into the LAA. For example, and without limitation, FIG. 2G shows the arrangement of treatment device 100 of FIG. 2A advanced the left atrium, the implant device 102 being in a collapsed state and restrained within an outer tube of the catheter. FIG. 2H shows the contact member 104 being partially or completely expanded (or partially or completely moved to the second state) within the left atrium before being advanced into the LAA. As shown in FIG. 2I, the contact member 104 and other components of the treatment device 100 can be advanced into the LAA when the contact member is in an expanded or second state, or when the contact member is partially in an expanded state or is between the first state and the second state. As shown in FIG. 2J, the contact member can be rotated to twist the LAA and cause a neck or opening of the LAA to constrict around a portion of the implant device, just as described above.

Other steps to complete the treatment can be as described above and in other methods disclosed herein. For instance, as noted above, and as described in greater detail below, in some instances of use, the physician may rotate contact member 104 through a first range of motion in the first direction (as shown in FIG. 2J), then pull contact member 104 proximally a certain distance, then rotate contact member 104 through a second range of motion in the first direction. This combination of rotational movement and translational movement of contact member 104 may provide a combination of torsional compression and longitudinal compression of the tissue of the LAA. In some such uses, the second range of rotation may be less than the first range of motion. By way of example only, the second range of rotation may be approximately one third of the first range of motion. By way of further example only, contact member 104 may be pulled in the proximal direction along a distance that is approximately equal to the length of contact member between the stage of rotating contact member 104 through the first range of motion and the stage of rotating contact member 104 through the second range of motion

Note that, as mentioned above, any of the treatment device arrangements disclosed herein can be configured so that the contact member can be partially or completely expanded in the left atrium before the contact member is advanced into the LAA. Similarly, in any of the arrangements of the methods disclosed herein (For example, and without limitation, the arrangements of treating and/or occluding the LAA), the contact member can be partially or completely expanded in the left atrium before the contact member is advanced into the LAA. In certain arrangements, the contact member is not further expanded once positioned within the LAA and, in certain arrangements, the contact member can be further expanded or constricted once positioned within the LAA. In certain arrangements, the contact member could be constricted in the left atrium before entering the LAA and then could remain in a constricted position within the LAA or could be further expanded or constricted once positioned within the LAA.

As noted above, the contact member can be rotated to twist the LAA so as to cause a neck or a portion of the LAA adjacent to the opening of the LAA to constrict and substantially or fully close about an outside surface of a portion of the implant device, thereby causing the opening of the LAA to be occluded. In the illustrated arrangement, the contact member 104 can be rotated about its longitudinal axis to cause the twisting of the LAA. In certain arrangements, the longitudinal axis that the contact member is rotated about can correspond to or be closely aligned with an insertion axis of the securing element 110 as it is advanced towards the contact member 104. Additionally, any of the arrangements of the methods and devices disclosed herein can be configured such that the implant or contact member can be advanced from the delivery catheter and engage a wall of the LAA without the implant or contact member completely or partially expanding, changing size, changing shape, or moving to or toward a second state. For example, in some arrangements, the implant or contact member can be configured to engage and, upon rotation of the implant or contact member, rotate the LAA without the implant or contact member completely or partially expanding, changing size, changing shape, or moving to or toward a second state.

FIG. 2K shows another arrangement of treatment device 100′ having an implant device 102′ being advanced through a catheter into the LAA, the implant device 102′ being in a collapsed state and restrained within an outer tube 114 of the catheter. FIG. 2L shows the arrangement of the implant device 102′ of FIG. 2K engaged with the patient's tissue that has constricted as a result of the twisting of the LAA. In any arrangements, the implant device 102′ can have a contact member 104′, a securing element 110′, and a retention element 108′ extending between the contact member 104′ and the securing element 110′. In some arrangements, the implant device 102′ can be flipped as compared to the implant device 102 described above.

In some arrangements, the contact member 104′ can be configured to treat the LAA the same as any other arrangements of the contact members disclosed herein. For example, and without limitation, the contact member 104′ can be configured to engage a tissue portion inside the LAA and twist the LAA so as to cause a portion of tissue of the LAA to constrict inwardly, just as other arrangements of the contact members disclosed herein. In the illustrated arrangement, the contact member 104′ can have the same or a similar structure, functionality, components, and/or other details as any of the arrangements of the securing elements disclosed herein, For example, and without limitation, the arrangements of the securing elements 110 disclosed herein, while being configured for engaging the tissue inside the LAA and twisting the LAA to constrict and/or occlude the ostium of the LAA.

Further, in some arrangements, the securing element 110′ can be configured to treat the LAA the same as any other arrangements of the securing elements disclosed herein. For example, and without limitation, the securing element 110′ can be configured to engage the tissue that has constricted as a result of the twisting of the LAA so as to inhibit the constricted tissue from untwisting and/or so as to inhibit the constricted opening of the LAA from expanding. In the illustrated arrangement, the securing element 110′ can have the same or a similar structure and functionality as any of the arrangements of the contact members disclosed herein, For example, and without limitation, the arrangements of the contact members 104 disclosed herein.

In other arrangements, the implant device 110′ can have a contact member that is similar to the arrangements of the contact member 104 disclosed herein or other arrangements of contact members disclosed herein (with the exception of the arrangements of the contact member 104′) along with the arrangements of the securing element 110′ disclosed herein, or a securing element that has a structure that is the same or similar to any other arrangements of contact members disclosed herein (with the exception of the arrangements of the contact member 104′). Alternatively, in other arrangements, the implant device 110′ can have a contact member 104′ as disclosed herein and can have a securing element that is similar to any of the other securing elements shown herein, such as any of the arrangements of the securing element 110 disclosed herein.

Any of the components of any of the implant arrangements disclosed herein can be made from Nitinol or any other elastic or super elastic material, including any other shape memory materials, or any mechanically expandable material such as stainless steel or otherwise. In any arrangements disclosed herein, the contact member (such as contact member 104) can have a spherical, cylindrical, or other shape, such as the shape of an elongated bullet, a stent, a mushroom, or other non-round or non-cylindrical shape or any of the shapes described or shown with respect to any of the arrangements disclosed herein. In any arrangements disclosed herein, the contact member may comprise a series of interconnected struts (that can, but are not required to, form a diamond shaped pattern across all or a portion of the surface of the contact member), or may be made from a series of ribs or paddles which form the expandable device.

### II. Examples of LAA Treatment Device

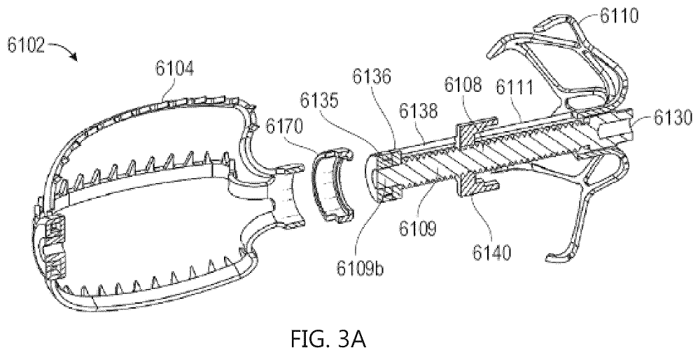

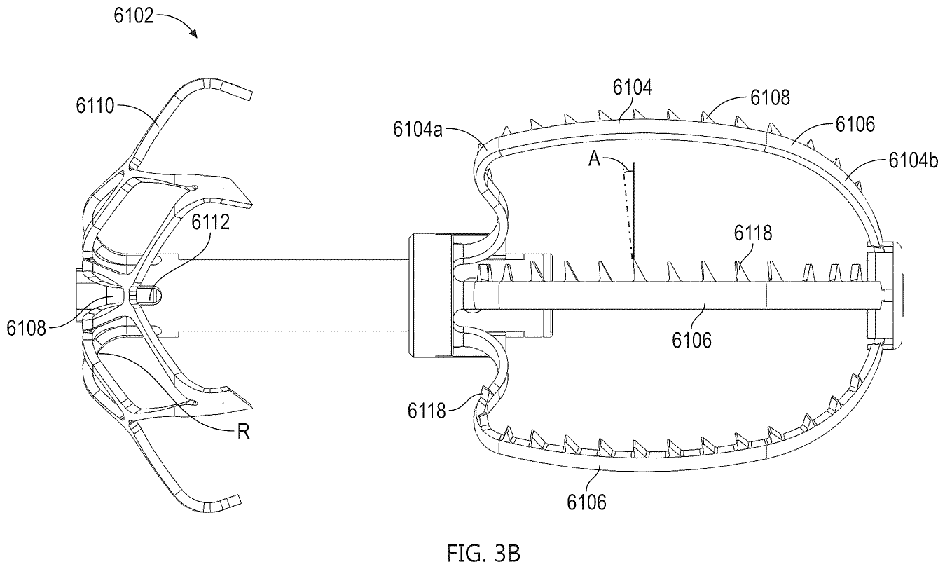

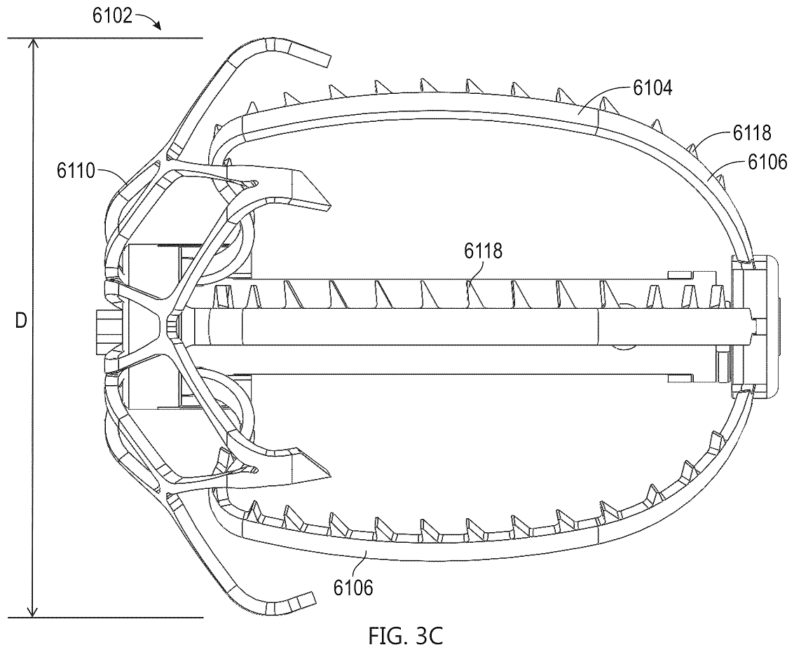

FIGS. 3A-3C show another arrangement of a treatment device 6100 having an implant 6102 having a contact member 6104, and retention element 6108, and securing element 6110. In any arrangements disclosed herein, the implant device 6102 can have any of the components, features, or other details of any other treatment device arrangements or implant device arrangements disclosed herein, including without limitation any of the other arrangements of the treatment devices or systems 100, 140, 4000, 6000 or implant devices 102, 104, 4002, 6002 described herein, in any combination with any of the components, features, or details of the implant device 6102 shown in FIGS. 3A-3C. Similarly, any components, features, or other details of any of the other treatment device arrangements or implant device arrangements disclosed herein can have any of the components, features, or other details of any arrangements of the implant device 6102 disclosed herein in any combination with any of the components, features, or details of the other arrangements of the treatment device and/or implant device disclosed herein.

With reference to FIGS. 3A-3C, any arrangements of the contact member 6104 disclosed herein can have a plurality of tissue anchors or teeth 6118 (also referred to as nubs) or other similar features configured to penetrate or engage the tissue of the LAA. The tissue anchors 6118 can be configured to penetrate into a tissue within the LAA when the contact member 4004 is expanded against the tissue of the LAA and/or when the contact member 4004 is rotated or twisted within the LAA. The tissue anchors 6118 can be positioned on one side of the struts 6106 of the contact member 6104, for example, to point in the direction of intended rotation of the contact member 6104. In other arrangements, the tissue anchors 6118 can be positioned on both sides of the struts 6106 of the contact member.

In any arrangements disclosed herein, the tissue anchors or teeth 6118 of the contact member can be asymmetrical or otherwise be formed at an angle (such as angle A, shown in FIG. 3C). In some arrangements, as shown, the tissue anchors 6118 can be directed toward a proximal end 6104a of the contact member 6104 by an angle A. In this arrangement, in some arrangements, the tissue anchors can be angled toward an ostium. For example, and without limitation, the anchors can have a proximal surface that is angled back toward the proximal end of the contact member by an angle A of 5° or approximately 5°, or from 2° or approximately 2° to 15° or approximately 15° or more, or from 5° or approximately 5° to 10° or approximately 10°, or of any value within the foregoing ranges or to and from any values within the foregoing ranges. In some arrangements, angling the tissue anchors toward the proximal end of the contact member can improve the engagement (e.g., grip) of the tissue anchors in the tissue of the LAA as the retention element and/or the securing element are drawn toward the contact member, which can cause the contact member to be pulled toward the ostium of the LAA. In other arrangements, the tissue anchors 6118 can have a distal surface that can be angled toward a distal end 6104b of the contact member 6104, or can have a mix of tissue anchors having a proximal surface angled toward the proximal end of the contact member, tissue anchors having a distal surface angled toward the distal end of the contact member, and/or symmetrically shaped tissue anchors.

In some arrangements, a length of any of the tissue anchors disclosed herein (For example, and without limitation, the tissue anchors 6118), measured from the base of the tissue anchor to a distal tip of the tissue anchor along a centerline of the tissue anchor, can be 0.6 mm, or approximately 0.6 mm. In some arrangements, the length of the tissue anchors can be 0.5 mm, or from 0.4 mm (or approximately 0.4 mm, or less than 0.4 mm) to 0.8 mm (or approximately 0.8 mm, or more than 0.8 mm), or from 0.5 mm (or approximately 0.5 mm) to 0.7 mm (or approximately 0.7 mm), or of any value or range of values within any of the foregoing ranges.

With reference to FIGS. 3A-3C, in some arrangements, the tissue anchors 6118 can be positioned along a length of the struts 6108 of the contact member 6104 from the proximal end 6104a of the contact member 6104 to the distal end 6104b of the contact member 6104, or near or adjacent to the proximal end 6104a of the contact member 6104 to a point that is adjacent to or near to the distal end 6104b of the contact member 6104. In some arrangements, the tissue anchors 6118 can be positioned along at least 80% of a length of the struts 6108 of the contact member 6104, or from 60% (or approximately 60%, or less than 60%) to 100% (or approximately 100%) of the length of the struts 6108 of the contact member 6104, or of any values or ranges of values within the foregoing ranges.

The securing element 6110 shown in FIGS. 3A-3C is shown in an expanded state (e.g., is in the second state) and spaced apart from the contact member 6104 that is also in an expanded, second state. In some arrangements of the implant 6102, similar to the implant 6002 described above, the securing element 6110 can be axially secured to the retention element 6108. Some arrangements of the retention element 6108 can have a threaded shaft that can be positioned within a body portion of the securing element 6110. The threaded shaft 6109 can be permitted to rotate freely within the body portion 6111 of the securing element 6110. The threaded shaft 6109 can also be threadedly coupled with the contact member 6104. In this configuration, rotating the threaded shaft 6109 in a first direction can cause the securing element 6110 to advance axially toward the contact member 6104. Rotating the threaded shaft 6109 in a second direction which is opposite to the first direction can cause the securing element 6110 to withdraw or move axially away from the contact member 6104. With reference to FIG. 3C, the securing element 6110 can have a bend radius or radius of curvature (represented by R in FIG. 3C) near a base of the struts of the securing element 6110 that can be 1.0 mm (or approximately 1.0 mm) in size, or from 0.8 mm (or approximately 0.8 mm, or less than 0.8 mm) to 1.0 mm (or approximately 1.0 mm, or more than 1.0 mm), or from 0.6 mm (or approximately 0.6 mm, or less than 0.6 mm) to 1.4 mm (or approximately 1.4 mm, or more than 1.4 mm), or of any values or ranges of values within the foregoing ranges. In any arrangements disclosed herein, the securing element (including securing element 6110) can have an overall outside diameter (represented by D in FIG. 3C) of 13 mm (or approximately 13 mm), or from 10 mm (or approximately 10 mm, or less than 10 mm) to 20 mm (or approximately 20 mm, or more than 20 mm), or from 12 mm (or approximately 12 mm) to 17 mm (or approximately 17 mm), or of any values or ranges of values within the foregoing ranges.

In some arrangements, with reference to FIG. 3C, the space 6112 between the struts (also referred to herein as arms) of the securing element 6110 can be configured to reduce the stress in the struts and allow for a better stress and/or strain distribution along a length of the base portion of the struts of the securing element 6110. By increasing the length of the space between the struts of the securing element 6110, the struts are able to bend or flex more in the base portion of the struts, therein optimizing the stress and/or strain distribution along a length of the base portion of the struts.

In some arrangements, the retention element 6108 can have a head 6130 coupled with (e.g., integrally formed with) the threaded shaft 6109, the head 6130 being configured to couple with an end portion of an intermediate member of the catheter (not shown) so that a rotation or torque applied to the intermediate member can cause an equal rotation or torque to be applied to the head 6130 and the threaded shaft 6109 of the retention element 6108. In some arrangements, the retention element 6108 can be axially coupled with the body portion 6111 of the securing element 6110 so that the retention element 6108 and the securing element 6110 move together in either axial direction. For example, and without limitation, in some arrangements, the retention element 6108 can have a first retainer 6135 that can be coupled with (e.g., welded to, press fit, or otherwise attached to) a distal end 6109b of the threaded shaft 6109 and a second retainer 6136 that can be positioned within and be axially constrained within the slot 6138 formed in the body portion 6111 of the securing element 6110.

The first retainer 6135 can prevent a proximal axial movement of the threaded shaft 6109 (i.e., in the proximal direction, away from the contact member 6104) relative to the body portion 6111 of the securing element 6110. Because the second retainer 6136 can have an opening axially therethrough that is smaller than the major diameter of the threaded portion of the threaded shaft 6108, the second retainer 6136 can prevent a distal axial movement of the threaded shaft 6109 (e.g., in the distal direction, toward the contact member 6104) relative to the body portion 6111 of the securing element 6110. In this configuration, any axial movement of the retention element 6108 will cause the simultaneous and equal axial movement of the securing element 6110. A proximal end portion of the contact member 6104 can have a collar member 6170. In some arrangements, the collar member 6170 can be used to constrain the post 6140 to the contact member 6104 so that the post 6140 cannot become disengaged from the contact member 6104.

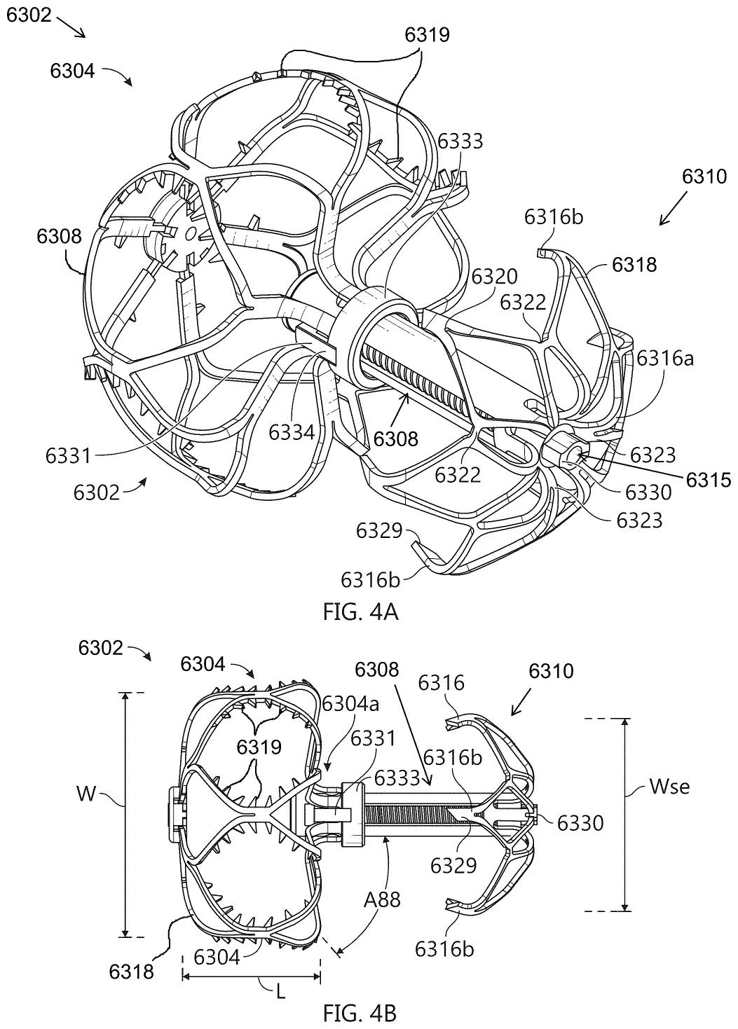

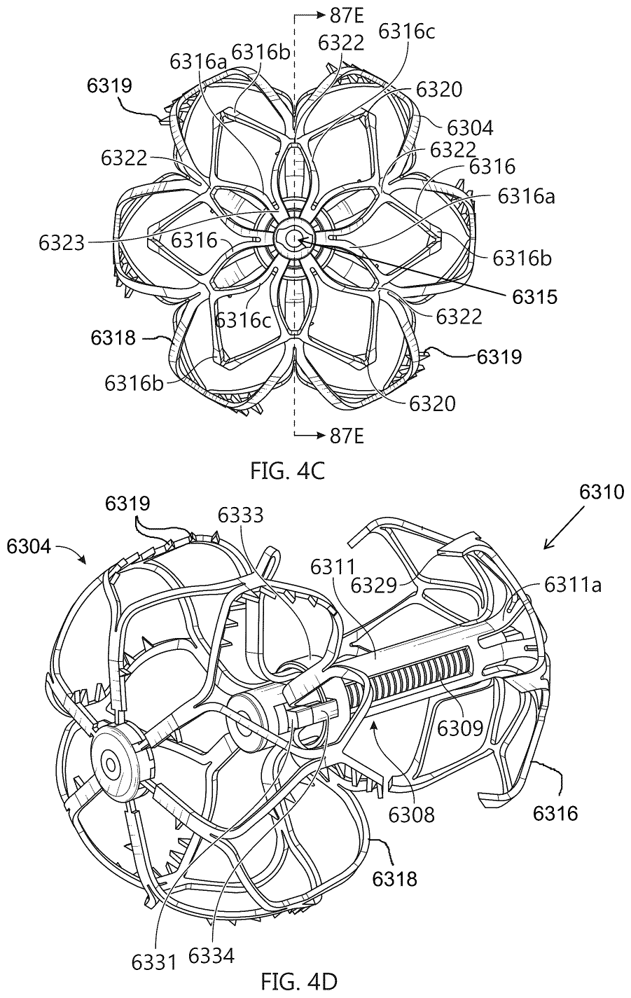

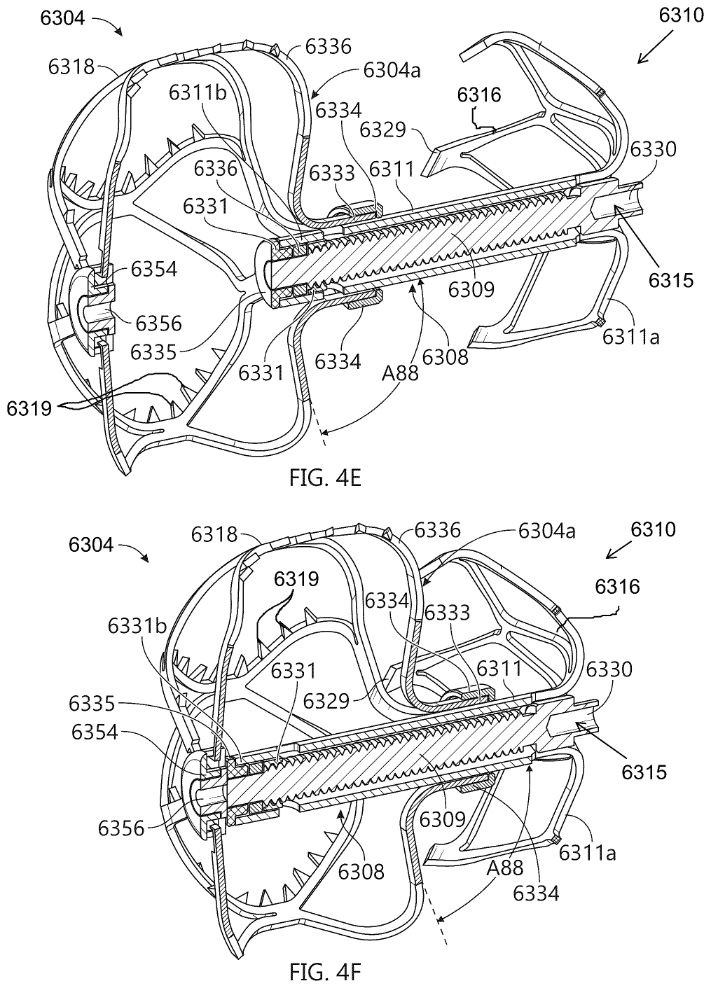

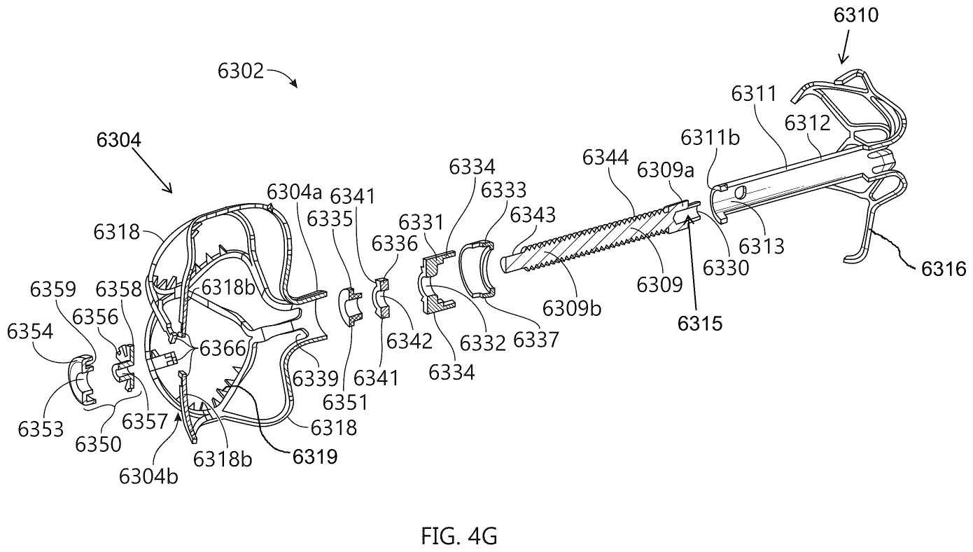

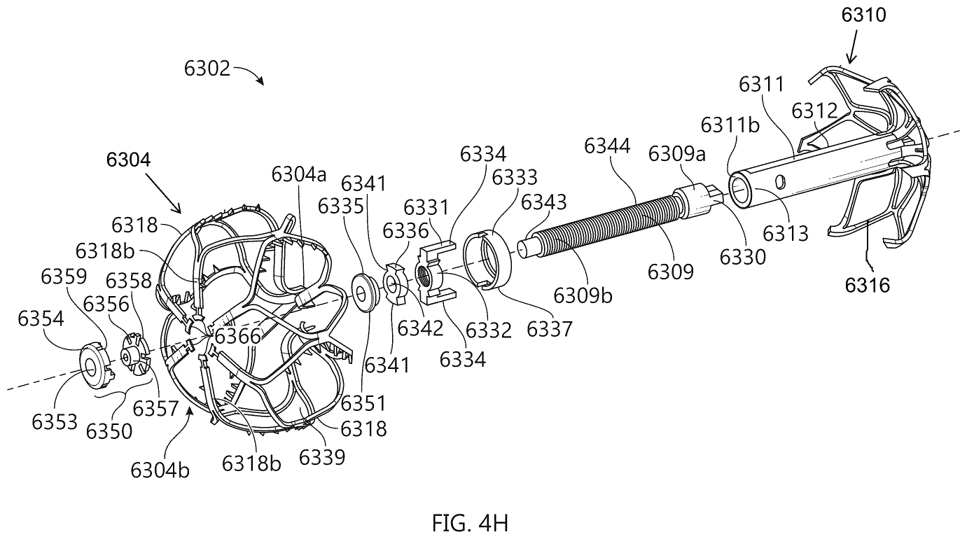

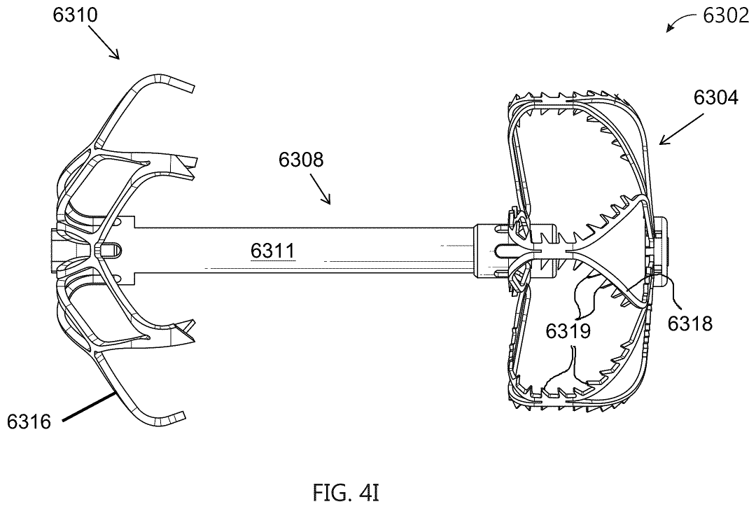

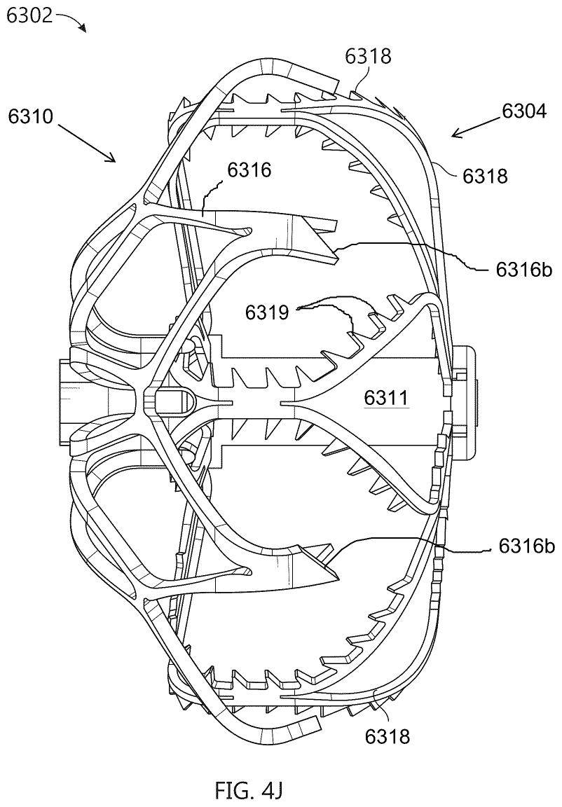

FIGS. 4A-4J show another arrangement of a treatment system 6300 having an arrangement of an implant device 6302 having a contact member 6304, a retention element 6308, and a securing element 6310. In any arrangements disclosed herein, the treatment system 6300 and/or implant device 6302 can have any of the components, features, or other details of any other treatment device arrangements or implant device arrangements disclosed herein, including without limitation any of the other arrangements of the treatment devices 100, 140, 4000, 6000, 6100 and/or implant devices 102, 104, 4002, 6002, 6102 disclosed herein, in any combination with any of the components, features, or details of the treatment system 6300 and/or the implant device 6302 shown in FIGS. 4A-4J. Similarly, any components, features, or other details of any of the other treatment device arrangements or implant device arrangements disclosed herein can have any of the components, features, or other details of any arrangements of the treatment device 6300 and/or implant device 6302 disclosed herein in any combination with any of the components, features, or details of the other arrangements of the treatment device and/or implant device disclosed herein.

FIG. 4A shows an isometric view of the implant device 6302, in which the securing element 6310 that has been expanded (e.g., is in the second state) is spaced apart from the contact member 6304 that is also in an expanded, second state. FIG. 4B shows a side view of the implant device 6302 and the expanded securing element 6310 spaced apart from the implant device 6302. FIG. 4C shows an end view of the implant device 6302.

With reference to FIGS. 4A-4J, in some arrangements of the implant device 6302, the securing element 6310 can be axially coupled with or secured to a retention element 6308. In the present example, retention element 6308 has a threaded shaft 6309 that can be positioned within a body portion 6311 of the securing element 6310 in an operable state. Retention element 6308 may thus be viewed as a combination of threaded shaft 6309 and body portion 6311. The body portion 6311 can have a cylindrical shape with an opening axially therethrough that can be sized and configured to receive the threaded shaft 6309 therein. In some arrangements, the threaded shaft 6309 can be permitted to rotate freely within the body portion 6311 of the securing element 6310 in either direction. The threaded shaft 6309 can also be threadedly coupled with the contact member 6304. In this configuration, rotating the threaded shaft 6309 in a first direction can cause the securing element 6310 to advance axially toward the contact member 6304 and rotating the threaded shaft 6309 in a second, opposite direction can cause the securing element 6310 to move axially away from the contact member 6304.

Threading of threaded shaft 6309 may have a fine pitch, to provide a self-locking relationship between threaded shaft 6309 and body portion 6311 of the securing element 6310, to prevent inadvertent unscrewing of threaded shaft 6309 from body portion 6311 (e.g., in response to movement caused by the patient's heart beating after implant device 6302 has been installed in a patient). By way of further example only, threaded shaft 6309 may have a diameter of approximately 2 mm (0.079 in) or any other suitable diameter.

Retention element 6308 may comprise titanium and/or any other suitable material(s). In some versions, to minimize the risk of galling from part-to-part interactions, retention element 6308 comprises a first bio-compatible metal (e.g., titanium, etc.) while other parts with which retention element 6308 interacts (e.g., contact member 6304, etc.) comprise a one or more other bio-compatible metals (e.g., nitinol, stainless steel, etc.).

FIG. 4F is a section view of the arrangement of the implant device 6302 shown in FIG. 4A, showing the securing element 6310 advanced to a position that is adjacent to the contact member 6304. FIG. 4G is an exploded section view of the arrangement of the implant device 6302 shown in FIG. 4A. FIG. 4J is a side view of the arrangement of the implant shown in FIG. 4A, also showing the securing element 6310 advanced to a position that is adjacent to the contact member 6304. Some arrangements of implant device 6302 may be configured to permit the securing element 6310 to advance to a position that is adjacent to the contact member 6304 such that distal end portions 6316b of struts 6316 of the securing element 6310 extend past the proximal end of the contact member 6304, as shown in FIG. 4J. However, for some arrangements, when implanted in the body, some tissue of the left atrium and/or the LAA may be positioned between the securing element 6310 and the contact member 6304, which may limit the range of movement of the securing element 6310 relative to the contact member 6304. As mentioned, rotating the threaded shaft 6309 in a second direction which is opposite to the first direction can cause the securing element 6310 to retract proximally or move axially away from the contact member 6304. FIGS. 4E and 4H show the securing element 6310 advanced to a position that is spaced apart from the contact member 6304. In some arrangements, without limitation, the first direction can be clockwise.

In the present example, securing element 6310 has a plurality of arms or struts 6316 extending away from a proximal end portion 6311a of the body portion 6311 of the securing element 6310. In some arrangements, the plurality of struts 6316 can each initially bend radially outwardly at a proximal end portion 6316a thereof and can each have a distal end portion 6316b that can be, in the second or expanded state of the securing element 6310, closer to the contact member 6304 than the proximal end portion 6316a of the struts 6316. Each of the struts 6316 can have a middle section 6316c that, in a second or expanded state, can angle outwardly and forward toward the contact member. In some arrangements, the middle section 6316c can angle forward at an angle that is 45° (or approximately 45°) relative to an axial or longitudinal axis of the body portion 6311 of the securing element 6310, or from 35° (or approximately 35°, or less than 35°) to 60° (or approximately 60°, or more than 60°) relative to the axial or longitudinal axis of the body portion 6311.

With reference to FIG. 4J, in some arrangements, at least a distal end portion 6316b of some of the plurality of struts 6316 can overlap with the contact member 6304 when the securing element 6310 is advanced in the distal direction (e.g., when the securing element 6310 is moved to a position that is adjacent to the contact member 6304). For example, and without limitation, in any arrangements of implant device 6302 disclosed herein, at least a distal end portion 6316b of some of the plurality of struts 6316 can overlap a proximal end of the contact member by 1 mm, approximately 1 mm, or less than 1 mm, or 2 mm or approximately 2 mm, or 2 mm or approximately 2 mm, or from 1 mm or less than 1 mm to 3 mm or more than 3 mm. As another nonlimiting example, in any arrangements of the implant disclosed herein, at least a distal end portion 6316b of some of the plurality of struts 6316 can overlap a proximal end of the contact member 6304 such that at least 20% or approximately 20%, or at least 40% or approximately 40%, or at least 50% or approximately 50%, or from 20% or approximately 20% to 50% or approximately 50% of an overall length of at least a distal end portion 6316b of some of the plurality of struts 6316 in a deployed state can extend past a proximal end portion of the contact member. In some arrangements, implant device 6302 can be configured such that struts 6316 of securing element 6310 can extend into the spaces or voids between the arms or struts 6318 of the contact member 6304 when securing element 6310 has been advanced toward the contact member 6304.

In any arrangements of the securing element 6310 disclosed herein, each of the struts 6316 can have one or more interconnections 6320 with adjacent struts 6316 along a length of each of the struts 6316, or a plurality of the struts 6316. For example, and without limitation, with respect to FIG. 4C, each of the struts 6316 can have a first interconnection 6320 at an end portion 6316b of each of the struts 6316, wherein the first interconnection 6320 is an interconnection between the distal end portions 6316b of two adjacent struts 6316. Additionally, in some arrangements, each of the struts 6316 can also have a second interconnection 6322 in a middle portion 6316c of each of the struts 6316, wherein the second interconnection 6322 is an interconnection between the middle portions 6316c of two adjacent struts 6316. In this configuration, each of the struts 6316 of the securing element 6310 can have a first and a second interconnection along a length thereof with an adjacent strut 6316. Additionally, any arrangements of the securing element 6310 can have one or more interconnections 6323 at the proximal end portions 6316a of the struts 6316, or at the proximal end portions 6316a of a plurality of the struts 6316.

The interconnections 6320, 6322, 6323 can provide additional rigidity and strength to the entire securing element 6310. Additionally, in some arrangements, each of the interconnections 6320, 6322, 6323 can also provide an additional point of securement of each of the struts 6316 to the securing element 6310 so that, if a strut becomes fractured or broken, the first interconnection 6320 and/or the second interconnection 6322 can couple or secure the broken or fractured strut 6316 to the securing element 6310 and prevent the broken or fractured strut 6316 from breaking loose and moving into the patient's heart or blood stream.

Additionally, in some arrangements, each of the struts 6316 can have a free distal end portion 6316b where, in some arrangements, the distal end portion 6316b can have two struts 6316 that are coupled together at the distal end portion 6316b of struts 6316. In some arrangements, the distal end portion 6316b can have a sharp point that is designed to penetrate tissue. In some arrangements, the distal end portion 6316b can have a point that is configured to grab or engage the tissue without penetrating the tissue. Additionally, in some arrangements, the distal end portion 6316b of each of the struts 6316 can have a sloped or angled surface 6329 that can assist with the penetration of the distal end portion 6316b into the tissue. In some such versions, the slope or angle of surface 6329 is oriented to provide resistance to untwisting/unwrapping of the LAA after implant device 6302 has been deployed as described in greater detail below.

In some arrangements, the first and/or second interconnections 6320, 6322 can increase a rigidity of the securing element 6310, at least torsionally. In some arrangements, with the more torsionally rigid configuration having interconnections, the struts 6316 can be made thinner in cross-sectional size, which can improve tissue ingrowth into the securing element 6310 in some arrangements and/or reduce a weight of the securing element 6310. In some arrangements, without limitation, the cross-sectional area or size of the struts 6316 can be the same as or approximately the same as a 2-0 suture.

Contact member 6304 of the present example comprises a plurality of struts 6318. Struts 6318 include a plurality of tissue anchors 6319. Tissue anchors 6319 of this example may function similar to tissue anchors 6118 described above, such that tissue anchors 6319 may engage the wall of the LAA while contact member 6304 is rotated within the LAA when contact member 6304 is in an expanded state, such that rotation of contact member 6304 within the LAA will cause the LAA to twist as described herein; and such that proximal retraction of contact member 6304 within the LAA will cause the LAA to longitudinally compress as described herein.

As best seen in FIGS. 4A-6, each tissue anchor 6319 extends from its respective strut 6318 with an orientation having a tangential aspect. Such tangential aspect of the tissue anchor 6319 orientation is tangential relative to the central longitudinal axis of body portion 6311; and tangential relative to the axis about which contact member 6304 is rotated during operation as described in greater detail below. Such tangential aspect of the tissue anchor 6319 orientation promotes engagement between contact member 6304 and the tissue of the inner wall of the LAA as contact member 6304 is rotated during operation as described in greater detail below. Moreover, several of the tissue anchors 6319 extend from their respective strut 6318 with an orientation having a proximal aspect (in addition to having the tangential aspect). Such proximal aspect of the orientation of these tissue anchors 6319 promotes engagement between contact member 6304 and the tissue of the inner wall of the LAA as contact member 6304 is retracted proximally during operation as described in greater detail below.

In the present example, anchors 6319 have a generally triangular shape, yet anchors 6319 are not so sharp as to necessarily penetrate the tissue of the inner wall of the LAA during operation. Instead, anchors 6319 simply provide enough traction to engage tissue in a manner sufficient to provide the twisting/wrapping and longitudinal compression of the LAA as described herein. Anchors 6319 may have any suitable configuration, such as barbs, hooks, cleats, rough texture, other surface features or protrusions, a coating or activated adhesive, etc.

Any arrangements of the contact member 6304 disclosed herein can have one or more interconnections between adjacent struts 6318 along a length of each of the struts 6318, or a plurality of the struts 6318. For example, and without limitation, with respect to FIG. 4C, each of the struts 6318 can have a first interconnection at or adjacent to an end portion 6318b of each of the struts 6318, wherein the first interconnection is an interconnection between the distal end portions 6318b of two adjacent struts 6318. Additionally, in some arrangements, each of the struts 6318 can also have a second interconnection in a middle portion 6318c of each of the struts 6318, wherein the second interconnection is an interconnection between the middle portions 6318c of two adjacent struts 6318. In this configuration, each of the struts 6318 of the contact member 6304 can have a first and a second interconnection along a length thereof with an adjacent strut 6318. Additionally, any arrangements of the contact member 6304 can have one or more interconnections at the proximal end portions 6318a of the struts 6318, or at the proximal end portions 6318a of a plurality of the struts 6318.

The interconnections can provide additional rigidity and strength to the entire contact member 6304. Additionally, in some arrangements, each of the interconnections can also provide an additional point of securement of each of the struts 6318 to the contact member 6304 so that, if a strut becomes fractured or broken, the first interconnection, the second interconnection, and/or the interconnections that can be at the proximal end portion of the contact member 6304 can couple or secure the broken or fractured strut 6318 to the contact member 6304 and prevent the broken or fractured strut 6318 from breaking loose and moving into the patient's heart or blood stream.

In the present example, the retention element 6308 has a head 6330 coupled with the threaded shaft 6309, the head 6330 being configured to couple with an end portion 8098b of second inner catheter 8098 of delivery catheter 8010 so that a rotation or torque applied to second inner catheter 8098 can cause an equal rotation or torque to be applied to the head 6330 and the threaded shaft 6309 of the retention element 6308. In some arrangements, the retention element 6308 can be axially coupled with the body portion 6311 of the securing element 6310 so that the retention element 6308 and the securing element 6310 move together in either axial direction. For example, and without limitation, in some arrangements, the threaded shaft 6309 of the retention element 6308 can threadedly engage with a threaded drive element 6331 that can have a threaded opening 6332 axially therethrough that can be configured to receive the threaded shaft 6309 therethrough. As will be described, the drive element 6331 can be configured to be supported by the other components of the implant device 6302 so that the drive element 6331 is prevented from rotating. Further, some arrangements of the implant device 6302 can be configured to permit the drive element 6331 to move axially along the length of the body portion 6311 of the securing element 6310 as the threaded shaft 6309 is rotated. In this configuration, the drive element 6331 can be advanced axially in either direction by rotating the threaded shaft 6309.

A retainer cap 6333 can be used to couple the drive element 6331 to a proximal end 6304a of the contact member 6304. In some arrangements, the retainer cap 6333 can be coupled with (e.g., welded to, press fit with, or otherwise attached to) the drive element 6331 with a portion of the proximal end 6304a of the contact member 6304 trapped or secured between the drive element 6331 and the retainer cap 6333. For example, and without limitation, the drive element 6331 can be positioned within an opening 6339 in the proximal end 6304a of the contact member 6304, distal to an annular ring of the proximal end 6304a of the contact member 6304, and the retainer cap 6333 can be coupled with the drive element 6331 with the proximal end 6304a of the contact member 6304 captured between the drive element 6331 and the retainer cap 6333. In some arrangements, the drive element 6331 can have posts or tabs 6334 that can extend into the slots 6312 of the body portion 6311 of the securing element 6310 to inhibit (e.g., prevent) the drive element 6331 from rotating when the threaded shaft 6309 is rotated into the drive element 6331. Additionally, in some arrangements, the tabs 6334 can engage with recesses 6337 formed in the retainer cap 6333 to provide an overlap between the drive element 6331 and the retainer cap 6333 and thereby improve the connection between the drive element 6331 and the retainer cap 6333.

A first retainer 6335 can be coupled with (e.g., welded to, press fit, or otherwise attached to) a distal end 6309b of the threaded shaft 6309 and a second retainer 6336 can be used to capture the distal end portion 6311b of the body portion 6311 of the securing element 6310 between the first retainer 6335 and the second retainer 6336. The second retainer 6336 can have posts or tabs 6341 that can extend into the slots 6312 of the body portion 6311 of the securing element 6310 to prevent the second retainer 6336 from moving in a distal direction relative to the distal end portion 6311b of the body portion 6311. This can be achieved by sizing the opening 6342 axially through the second retainer 6336 to be slightly oversized as compared to the cylindrical end portion 6343 at the distal end portion 6309b of the threaded shaft 6309, while sizing the opening 6342 to be smaller than the threaded portion 6344 of the threaded shaft 6309 so that the threaded portion 6344 of the threaded shaft 6309 cannot extend through the opening 6342 in the second retainer 6336. In this arrangement, with the second retainer 6336 positioned around the cylindrical end portion 6343 at the distal end portion 6309b of the threaded shaft 6309, the distal end portion 6311b of the body portion 6311 of the securing element 6310 positioned between the first retainer 6335 and the second 6336, and the first retainer 6335 non-removably coupled with the cylindrical end portion 6343 of the threaded shaft 6309, the threaded shaft 6309 can be non-removably coupled with the securing element 6310.

As mentioned, the first retainer 6335 can prevent a proximal axial movement of the threaded shaft 6309 (i.e., in the proximal direction, away from the contact member 6304) relative to the body portion 6311 of the securing element 6310. Because the second retainer 6336 can have an opening 6342 axially therethrough that is smaller than the major diameter of the threaded portion of the threaded shaft 6309, the second retainer 6336 can prevent a distal axial movement of the threaded shaft 6309 (i.e., in the distal direction, toward the contact member 6304) relative to the body portion 6311 of the securing element 6310. An outside diameter or a flange 6351 of the first retainer 6335 can be greater than the diameter of the opening 6313 at the distal end portion 6311b of the body portion 6311 of the securing element 6310 to prevent the first retainer 6335, which is coupled with an end portion 6309b of the threaded shaft 6309, from passing through the opening 6313. In this configuration, any axial movement of the retention element 6308 (e.g., caused by a rotation of the threaded shaft 6309) will cause the simultaneous and equal axial movement of the securing element 6310 relative to the contact member 6304.

In this configuration, as the threaded shaft 6309 is rotated in a first direction, the drive element 6331 can move axially in a first direction (e.g., a distal axial direction) relative to the securing element 6310 and so that, as the threaded shaft 6309 is rotated in a second direction, which is opposite to the first direction, the drive element 6331 can move axially in a second direction (e.g., a proximal axial direction) that is opposite to the first direction relative to the securing element 6310. As mentioned, the drive element 6331 can be coupled with a proximal end portion 6304a of the contact member 6304 such that, as the drive element 6331 is moved axially relative to the securing element 6310, the securing element 6310 can simultaneously and equally move in an axial direction relative to the contact member 6304. In this configuration, the securing element 6310 can be moved toward or away from the contact member 6304 by rotating the head portion 6330 of the threaded shaft 6309 of the retention element 6308.

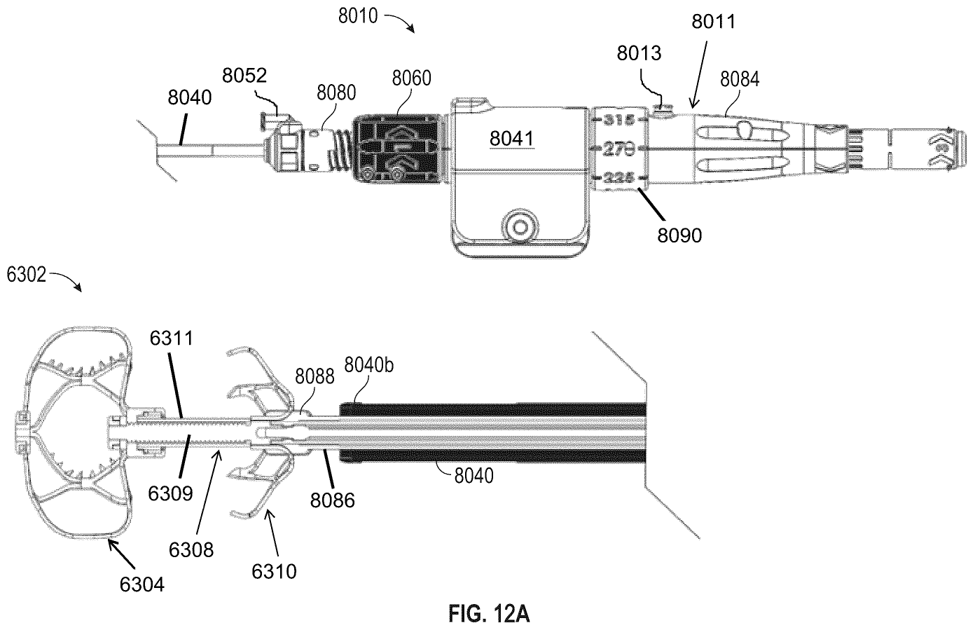

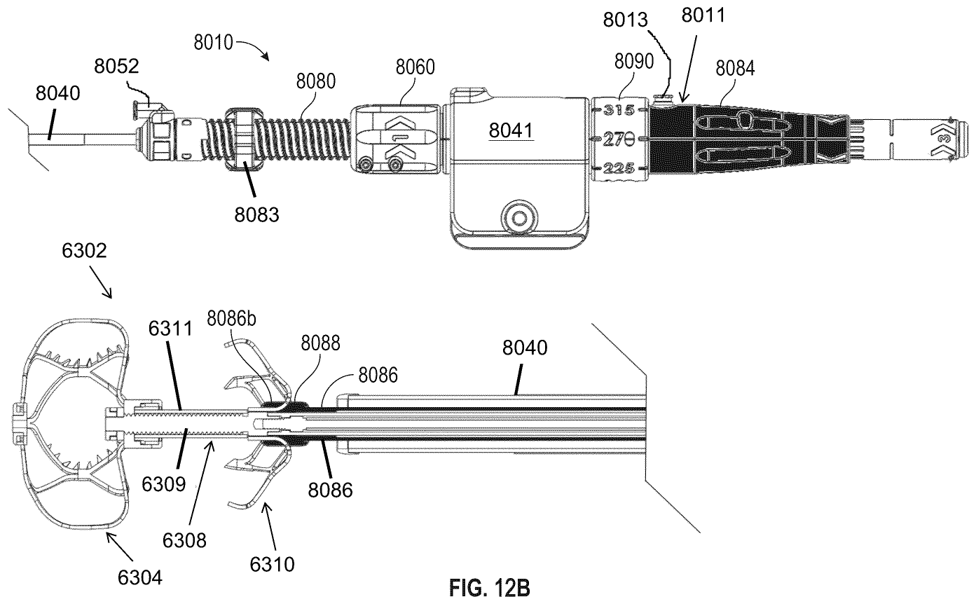

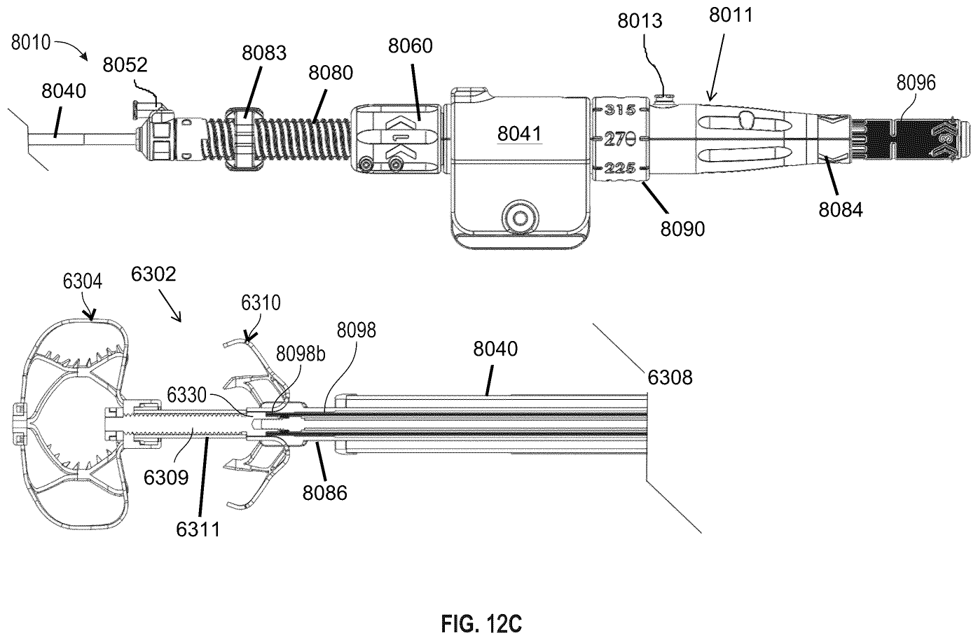

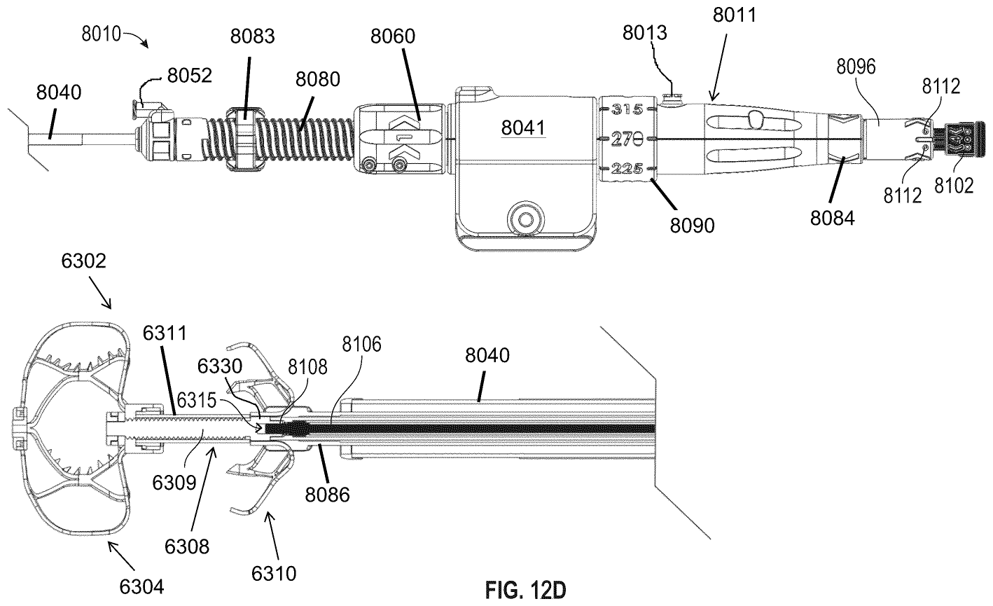

The proximal end of retention element 6308 further includes a threaded opening 6315 having internal threads. Threaded opening 6315 is formed in head 6330 of retention element. As described in greater detail below with reference to FIG. 12D, threaded opening 6315 is configured to threadedly engage with a threaded end portion 8108 of an inner core 8106 that is positioned inside of the second inner catheter 8098 to thereby longitudinally secure implant device 6302 with delivery catheter 8010. As also described below, threaded end portion 8108 is unthreaded from threaded opening 6315 to thereby release implant device 6302 from delivery catheter 8010. Before release of implant device 6302 from delivery catheter 8010 is desired, engagement between head and end portion 8098b of second inner catheter 8098 of delivery catheter 8010 prevents threaded end portion 8108 from being inadvertently unthreaded from threaded opening 6315. In some versions, threaded opening 6315 and threaded end portion 8108 are configured to handle tensile loads of at least approximately 20 pounds; or at least approximately 50 pounds.

With reference to FIG. 4G, some arrangements of the contact member 6304 can have a hub 6350 at a distal end portion 6304b of the contact member 6304 that can couple with the distal end portions 6318b of the struts 6318. The hub 6350 can couple with the distal end portions 6318b of the struts 6318 so as to secure the hub 6350 with the distal end portions 6318b of the struts 6318 in an axial direction. The hub 6350 can be configured to permit the distal end portions 6318b of the struts 6318 to rotate relative to the hub 6350. In some arrangements, the hub 6350 can include a first retention element 6354 and a second retention element 6356. The first retention element 6354 and a second retention element 6356 can have openings 6353 and 6357, respectively, axially therethrough for a guidewire or other instrument to pass through and can be configured to constrain the distal end portions 6318b of the struts 6318 in an assembled state. For example, and without limitation, the first retention element 6354 can have an annular recess therein that can be sized and configured to receive the distal end portions 6318b of the struts 6318 therein and the second retention element 6356 can have slots 6358 formed in the second retention element 6354 that extend in a radial direction and are sized and configured so that the thinned or narrow portion of the distal end portions 6318b of the struts 6318 can pass therethrough, but the wider portion at the distalmost end portion of the distal end portions 6318b of the struts 6318 cannot pass therethrough. In other words, the slots 6358 can be configured to engage with the tabs or other T-shaped features 6366 formed on the distal end portion 6318b of the struts 6318 and to permit the distal end portions 6318b to rotate relative to the hub 6350. The second retention element 6356 can also have slots or recesses therein that are configured to engage with tabs formed on the first retention element 6354 to provide a more secure coupling between the first retention element 6354 and the second retention element 6356.

In this configuration, as the threaded shaft 6309 is rotated in a first direction, the securing element 6310 can be advanced toward the contact member 6304 so as to engage with and/or compress any tissue that has constricted or closed as a result of the twisting of the contact member 6304 or the LAA and/or any tissue that is adjacent to the tissue that has constricted or closed as a result of the twisting of the contact member 6304 or the LAA. The distal tips 6316b of the arms 6316 of the securing element 6310 can penetrate into the tissue that has been compressed or otherwise inhibit (e.g., without limitation, prevent) the tissue that has constricted around a body of the implant device 6302 from opening back up or expanding.

In some arrangements and as described above, the proximal end portion 6304a of the contact member 6304 and distal end portion 6304b of the contact member 6304 can be formed by struts. In some arrangements, For example, and without limitation, the proximal portion of the contact member 6304 or any contact member disclosed herein can be configured to bias any folds or overlapped tissue of the LAA that has formed around the contact member 6304 as a result of the twisting of the contact member 6304 to slide off or move away from the contact member 6304, so that only a minimal amount of folds or overlapped tissue, if any, will be formed around the outside of the contact member 6304. For example, and without limitation, in some arrangements, the proximal end portion 6304a of the contact member 6304 or any struts thereof can be angled (see angle A88 shown in FIG. 4B) at 85° or approximately 85° relative to a longitudinal axis of the contact member 6304, or at 90° or approximately 90° relative to a longitudinal axis of the contact member 6304, or from 80° or approximately 80° to 100° or approximately 100° relative to a longitudinal axis of the contact member 6304. In any arrangements, the proximal end portion 6304a of the contact member 6304 or any struts thereof and/or the distal end portion 6304b of the contact member 6304 or any struts thereof can be angled within 30° or approximately 30°, or within from 10° or approximately 10° to 30° or approximately 30°, of an angle that is normal to the longitudinal axis or longitudinal centerline of the contact member 6304. Further, in any arrangements, the mid portion of the contact member 6304 and/or any struts thereof can be approximately parallel with the longitudinal centerline axis of the contact member 6304, or within 30° or approximately 30°, or within from 10° or approximately 10° to 30° or approximately 30°, of parallel with the longitudinal axis of the contact member 6304

Accordingly, in some arrangements, the contact member 6304 can be configured so as to bias the folds or overlapping tissue to occur around the outside of the proximal end portion 6304a of the contact member 6304 and/or the body portion 6311 of the retention element 6308, at least after a threshold degree of rotation of the contact member 6304 relative to the LAA (e.g., after 90° or approximately 90° of rotation of the contact member 6304, or from 70° (or approximately 70°) to 110° (or approximately 110°) of rotation of the contact member 6304. In some arrangements, this can be achieved by using a contact member 6304 that has a lateral width or diameter that is equal to or only a little less than an inside size or diameter of the LAA at or adjacent to an end portion of the LAA. For example, and without limitation, in any arrangements of the implants disclosed herein, the contact member can have an outer size or diameter that is 80% or approximately 80%, or at least 80%, of an inside size or diameter of the LAA at or adjacent to an end portion of the LAA, or from 70% or approximately 70% to 100% or approximately 100% or greater than 100% of the inside size or diameter of the LAA at or adjacent to an end portion of the LAA, or from 80% or approximately 80% to 90% or approximately 90% of the inside size or diameter of the LAA at or adjacent to an end portion of the LAA.

As noted above, some versions of contact member 6304 comprise nitinol, though any other suitable material(s) may be used. By way of further example only, contact member 6304 may have a wall thickness ranging from approximately 0.15 mm (0.0059 in) to approximately 0.40 mm (0.0157 in); or more particularly, from approximately 0.20 mm (0.0079 in) to approximately 0.35 mm (0.0138 in). Such wall thickness may have sufficient structural integrity to provide the results described herein while also minimizing risk of plastic deformation while contact member 6304 is contained within delivery catheter assembly 8002. By way of further example only, securing element 6310 may also have a wall thickness as described above with respect to contact member 6304. Similarly, securing element 6310 may comprise nitinol and/or any other suitable material(s).

In any arrangements of the contact member 6304 disclosed herein, a length of the contact member 6304 along an outer surface of the contact member 6304 can be significantly less than a width or diameter of an outer surface of the contact member 6304. Having a shorter length, in some arrangements, can enable the body of the implant (e.g., the body portion of the retention element 6308, for example and without limitation) to have a greater length about which the tissue of the LAA can constrict to occlude or substantially occlude the LAA. In some arrangements, for example, and without limitation, a length of the contact member 6304 along an outer surface of the contact member 6304 (e.g., without limitation, as represented by length L in FIGS. 4B, 5, and 6) can be 50%, or approximately 50%, or less than 50% of a width or a diameter of an outer surface of the contact member 6304 (e.g., without limitation, as represented by width W in FIGS. 4B, 5, and 6), or from 40%, or approximately 40% to 60% or approximately 60% of a width or a diameter of an outer surface of the contact member 6304.

In some arrangements, the width or diameter of the outer surface of the contact member 6304 (e.g., without limitation, as represented by outer width W in FIGS. 4B, 5, and 6) can be greater than a width or a diameter of an outer surface of the securing element 6310 (e.g., without limitation, as represented by outer width Wse in FIGS. 4B, 5, and 6). For example, and without limitation, in some arrangements, the outer width W of the contact member 6304 can be from 50% or approximately 50% to 100% or approximately 100% greater than the outer width Wse of the securing element 6310. By way of further example only, the ratio of the outer width W of the contact member 6304 to the outer width Wse of the securing element 6310 may range from approximately 50% to approximately 175%; from approximately 60% to approximately 160%; from approximately 70% to approximately 145%; or from approximately 77% to approximately 138%.

In any arrangements disclosed herein, the outer width W of the contact member 6304 can be 12 mm (0.47 in) or approximately 12 mm (0.47 in), or can be 16 mm (0.63 in) or approximately 16 mm (0.63 in), or from 10 mm (0.39 in), approximately 10 mm (0.39 in), or less than 10 mm (0.39 in) to 18 mm (0.71 in), approximately 18 mm (0.71 in), or more than 18 mm (0.71 in), or any value or range of values in the foregoing range. In any arrangements disclosed herein, the outer width Wse of the securing element 6310 can be 13 mm (0.51 in) or approximately 13 mm (0.51 in), or can be from 10 mm (0.39 in), approximately 10 mm (0.39 in), or less than 10 mm (0.39 in) to 16 mm (0.63 in), approximately 16 mm (0.63 in), or more than 16 mm (0.63 in), or range from approximately 10 mm (0.39 in) to approximately 20 mm (0.79 in), or any value or range of values in the foregoing range.

It should also be understood that a securing element 6310 having the same outer width Wse may be used with different contact members 6304 having different respective outer widths, such that the outer width Wse of the securing element 6310 does not need to vary with the outer width W of the contact member 6304. For instance, a securing element 6310 having an outer width Wse of approximately 13 mm (0.51 in) may be used with a contact member 6304 having an outer width W of approximately 10 mm (0.39 in), with a contact member 6304 having an outer width W of approximately 12 mm (0.47 in), with a contact member 6304 having an outer width W of approximately 16 mm (0.63 in), or with a contact member 6304 having an outer width W of approximately 18 mm (0.71 in).

In some cases, the outer width Wse of the securing element 6310 may be substantially insensitive to the outer width W of the contact member 6304 because the securing element 6310 is only directly contacting the tissue of the left atrium left atrium outside the left atrial appendage LAA; whereas the contact member 6304 is only directly contacting the tissue within the left atrial appendage LAA. In other words, the securing element 6310 is not directly contacting the contact member 6304 in some versions, and there is otherwise no need for the outer width Wse of the securing element 6310 to equal or approximate the outer width W of the contact member 6304 in some such versions.

While the foregoing describes how the outer width Wse of securing element 6310 need not necessarily vary based on the outer width W of contact member 6304, in some versions of a securing element 6310, the lengths of struts 6316 may vary based on the outer width W of contact member 6304. For instance, the lengths of struts 6316 may be increased to account for an increase in the outer width W of contact member 6304. Similarly, in some cases, the length of retention element 6308 may vary with the outer width W of contact member 6304. For instance, the length of retention element 6308 may be increased to account for an increase in the outer width W of contact member 6304. This increase in length of retention element 6308 may accommodate additional tissue that may tend to wrap around retention element 6308 when a larger contact member 6304 is used in the procedures described herein. The increase in length of retention element 6308 may also provide a greater gap between distal end portions 6316b of struts 6316 and contact member 6304 to capture tissue.

There may also be some versions where the outer width Wse of the securing element varies based on the outer width W of the contact member 6304. By way of example only, the ratio of the outer width Wse of the securing element 6310 to the outer width W of the contact member 6304 may be approximately 1:1. By way of further example only, the ratio of the outer width Wse of the securing element 6310 to the outer width W of the contact member 6304 may range from approximately 1:2 to approximately 2:1. For instance, a contact member 6304 may have an outer width W of approximately 10 mm while a complementary securing element 6310 may have an outer width Wse of approximately 20 mm; or a contact member 6304 may have an outer width W of approximately 20 mm while a complementary securing element 6310 may have an outer width Wse of approximately 10 mm.

By way of further example, the length L of the expanded portion of the contact member 6304 may be 8 mm (0.31 in) or approximately 8 mm (0.31 in), or can be 9 mm (0.35 in) or approximately 9 mm (0.35 in), or from 8 mm (0.31 in), approximately 8 mm (0.31 in), or less than 8 mm (0.31 in) to 9 mm (0.35 in), approximately 9 mm (0.35 in), or more than 9 mm (0.35 in), or from approximately 5 mm (0.20 in) to approximately 15 mm (0.59 in), or any value or range of values in the foregoing range. Such lengths L of the expanded portion of the contact member 6304 may strike an appropriate balance between accommodating different LAA depths without having tissue wrap around retention element 6308 too quickly or too slowly when contact member 6304 is rotated within the LAA as described herein.

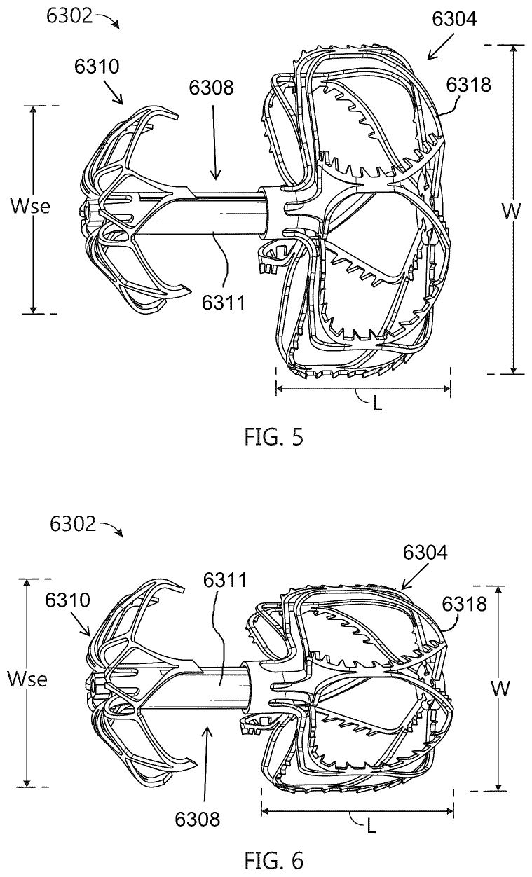

FIG. 5 shows an arrangement of a contact member 6304 having a lateral width or diameter that is a little less than (e.g., 10% or approximately 10% less than) an inside size or diameter of the LAA at or adjacent to an end portion of the LAA and a length along an outer surface of the contact member 6304 that is significantly less than the width or diameter of the outer surface of the contact member 6304. FIG. 4 shows the tissue of the LAA gathered around a body portion 6362 of the implant after the implant has been rotated approximately 360°. The simulated LAA has multiple folds 6366 in it around the body portion 6362 of the implant device 6302, but has far less folds, if any, in the simulated LAA tissue around the contact member 6304. In this arrangement, folds can begin forming in the LAA tissue in this arrangement or similarly sized and configured arrangements before the contact member 6304 is rotated 180°.

In any arrangements disclosed herein, the implant can be configured so that rotating the contact member 90° or approximately 90°, or from 60° or approximately 60° to 100° or approximately 100°, or from 70° or approximately 70° to 90° or approximately 90° from a first or initial position of the contact member can cause the contact member to contact and rotate at least an end portion of the tissue of the left atrial appendage (in some arrangements, For example, and without limitation, without forming any overlaps or folds or without forming a significant number of overlaps or folds in the tissue of the left atrial appendage around the contact member) and so that rotating the contact member from 90° or approximately 90° to 180° or approximately 180°, or from between 60° or approximately 60° and 100° or approximately 100° to between 160° or approximately 160° and 200° or approximately 200°, will cause the tissue of the left atrial appendage to twist around the implant at least between the contact member and the securing element. In some arrangements, the implant can be configured so that rotating the contact member past 180° or past approximately 180°, or past between 160° or approximately 160° to 200° or approximately 200°, can cause the tissue of the left atrial appendage to continue to twist and constrict around the implant mainly between the contact member and the securing element.

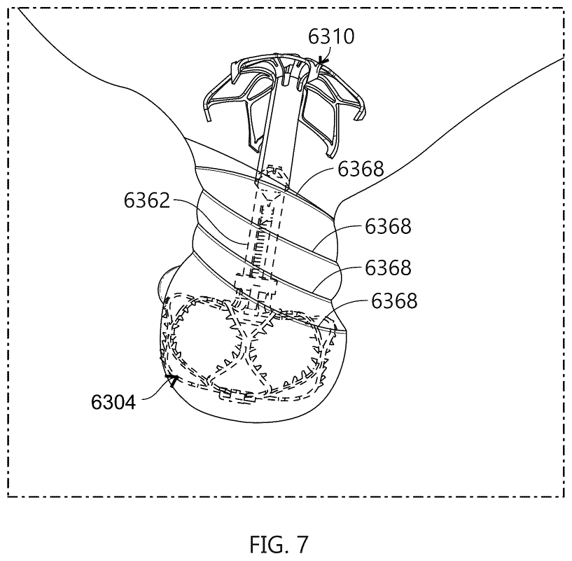

With some arrangements of the implant disclosed herein and, in some procedures, as the tissue is wrapped around the contact member 6304 and retention element 6308, the tissue can form one or more helical patterns 6368 of tissue, which can present one or more potential leak channels. This potential “folding” of the tissue is illustrated in FIG. 7, as discussed above. In some arrangements or procedures, the helical patterns 6368 can present leak channels after the wrapped tissue has been collapsed in an axial direction, which can cause the tissue to loosen around the outside surface of the implant device 6302. These leak channels, if present, and the angle of the leak channels relative to the axis of the contact member 6304 and retention element 6308 and delivery catheter, can be further compressed and reduced, respectively, with axial motion to change the angle of the channels to be closer to perpendicular relative to the longitudinal axis of the contact member 6304 and the delivery catheter and/or additional rotation of the contact member 6304 to further tighten the tissue around the outside surface of the contact member 6304 and retention element 6308. The additional rotation can cause the tissue to create a tighter seal around the contact member 6304 and retention element 6308. A nonlimiting example arrangement of steps that can be taken to achieve tighter compression of the tissue and a reduced angle of the folds relative to the longitudinal axis includes at least the following steps.

In any arrangements of the method of treatment or delivery disclosed herein, the procedure can include all or any of the following steps, in combination with any of the other steps or procedures disclosed herein. After the contact member 6304 has been advanced axially to the desired position within the LAA, the contact member 6304 can be rotated in a first direction by a first predetermined angle. For some arrangements, this can be done by rotating a second dial 8084 on the handle 8011 of the delivery catheter 8010. In any arrangements disclosed herein, the first predetermined angle can be greater than or equal to 180 degrees, or greater than approximately 180 degrees, or can be greater than or equal to 180 degrees, or greater than approximately 180 degrees, or can be 270 degrees, approximately 270 degrees, from 200 degrees or less than 200 degrees to 330 degrees or more than 330 degrees, from 230 degrees to 300 degrees, from 250 degrees to 290 degrees, or any value or range of values in any of the foregoing ranges. The user can then proximally withdraw (e.g., pull back) the contact member 6304 by a first predetermined distance. In some arrangements, the predetermined distance can be greater than or equal to 0.5 cm, or can be 1 cm, approximately 1 cm, from 0.25 cm to 1.75 cm, from 0.5 cm to 1.5 cm, from 0.75 cm to 1.25 cm, or any value or range of values in any of the foregoing ranges.

After the contact member 6304 has been withdrawn as described above, the contact member 6304 can then be rotated in the first direction by a second predetermined angle. For example, and without limitation, the second predetermined angle can be greater than or equal to 15 degrees, or can be greater than or equal to 30 degrees, or can be 65 degrees, approximately 65 degrees, from 30 degrees or less than 30 degrees to 90 degrees or more than 90 degrees, from 45 degrees to 150 degrees, from 30 degrees to 120 degrees, from 30 degrees to 90 degrees, or any value or range of values in any of the foregoing ranges. Thereafter, the securing element can be deployed and advanced toward the contact member.

Alternatively, in some arrangements, after rotating the contact member 6304 in the first direction by the second predetermined angle but before deploying the securing element 6310, the user can proximally withdraw (e.g., pull back) the contact member 6304 by a second predetermined distance. For example, and without limitation, the second predetermined distance can be 1 cm, approximately 1 cm, from 0.25 cm to 1.75 cm, from 0.5 cm to 1.5 cm, from 0.75 cm to 1.25 cm, or any value or range of values in any of the foregoing ranges. By way of further example only, the distance to which contact member 6304 is retracted proximally may be approximately the same as the length of contact member 6304. In some arrangements, the securing element 6310 can then be deployed and advanced toward the contact member 6304.

Alternatively, after withdrawing the contact member 6304 by the second predetermined distance and before deploying the securing element 6310, the contact member 6304 can then be rotated in the first direction by a third predetermined angle. For example, and without limitation, the third predetermined angle can be 30 degrees, approximately 30 degrees, from 15 degrees or less than 15 degrees to 60 degrees or more than 60 degrees, from 15 degrees to 100 degrees, from 15 degrees to 60 degrees, from 15 degrees to 45 degrees, or any value or range of values in any of the foregoing ranges. Thereafter, the securing element 6310 can be deployed and advanced toward the contact member.

### III. Example of LAA Treatment Device Delivery System

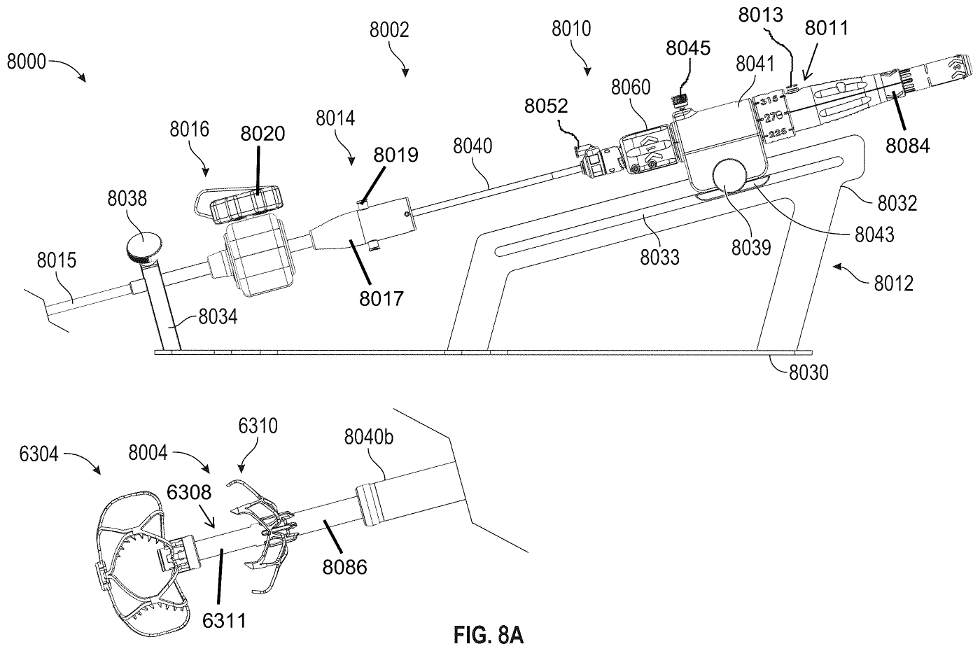

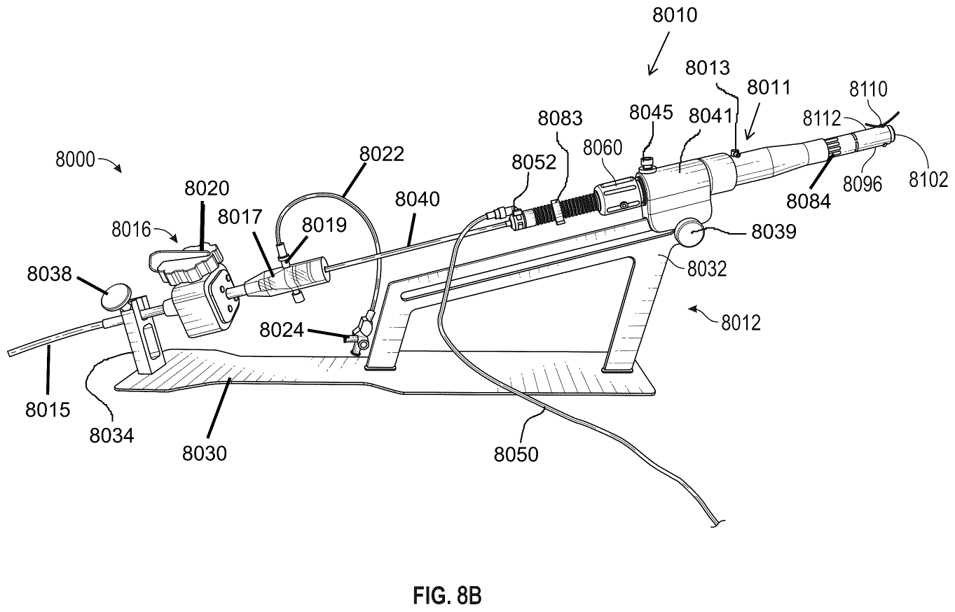

FIG. 8A shows an arrangement of a system 8000 for closing or occluding an LAA, including an arrangement of a delivery catheter assembly 8002 and an arrangement of an implant device 6302. FIG. 8B shows the arrangement of the delivery catheter assembly 8002 shown in FIG. 8A, including additional components for saline and contrast medium injection, etc. In any arrangements of the system 8000 disclosed herein, the implant device 6302 can be the same as or can have any combination of the features as any other arrangements of any of the implants disclosed herein, including without limitation implant 6102. For reference, the arrangement of the implant device 6302 is illustrated in FIG. 8A. As shown in FIGS. 8A and 8B, delivery catheter assembly 8002 of the present example includes a delivery catheter 8010, a support stand 8012, a guide catheter 8014, and a dilator (not shown). In the present example, the guide catheter 8014 includes a steerable guide sheath 8015 and a steering device 8016 having a steering knob 8020 to steer the steerable guide sheath 8015. In other arrangements, delivery catheter assembly 8002 can include a non-steerable guide catheter or sheath.

As also shown in FIGS. 8A-8B, the proximal end of the guide catheter 8014 of the present example includes a hub 8017. Hub 8017 includes a pair of luer ports 8019. Each luer port 8109 is in fluid communication with the interior of guide sheath 8015. As shown in FIG. 8B, a fluid conduit 8022 may be coupled with one of these luer ports 8019. In this example, the other end of fluid conduit 8022 includes a fitting 8024. Fitting 8024 of this example includes a set of three additional luer ports and a three-way stopcock valve to selectively couple each of these additional luer ports with fluid conduit 8022.

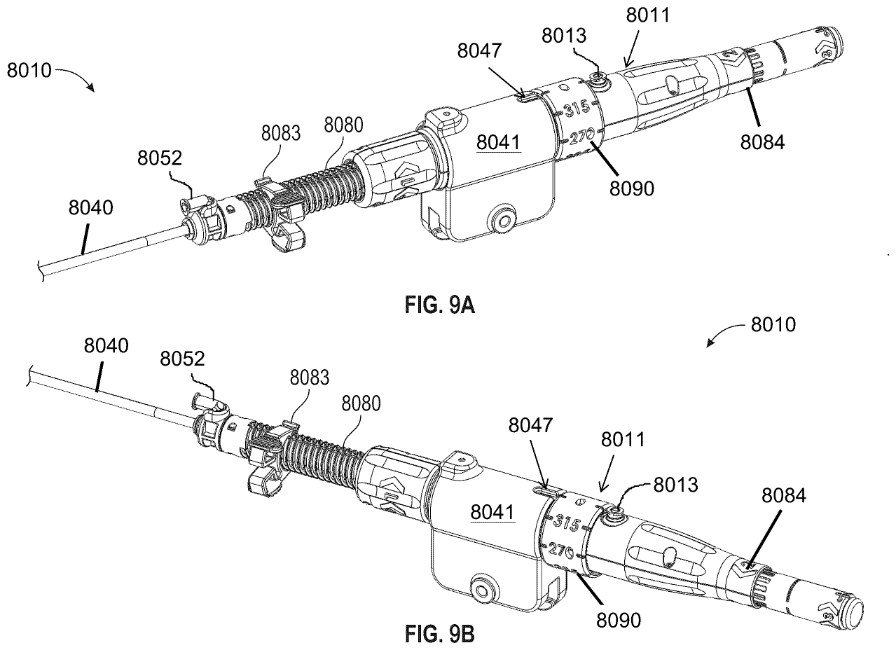

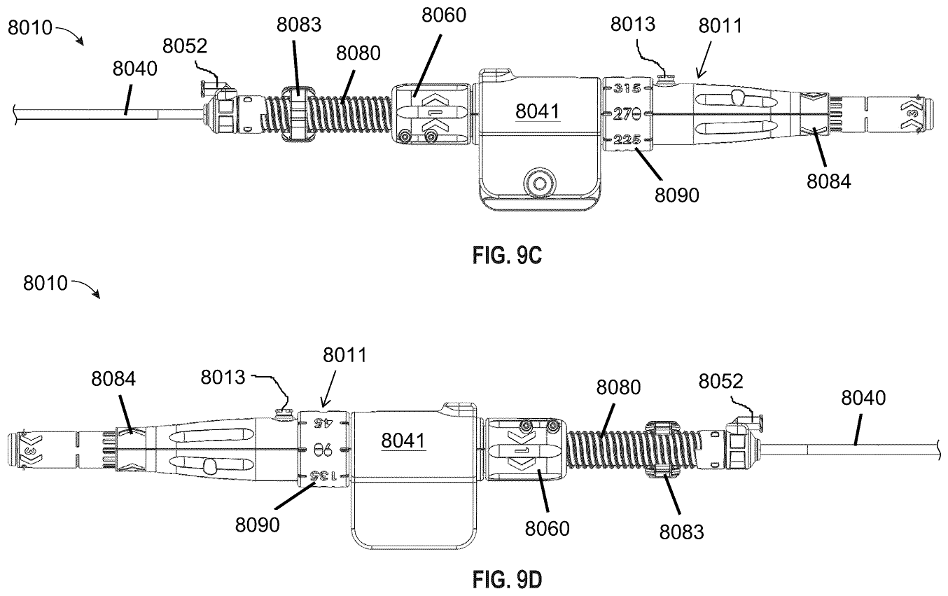

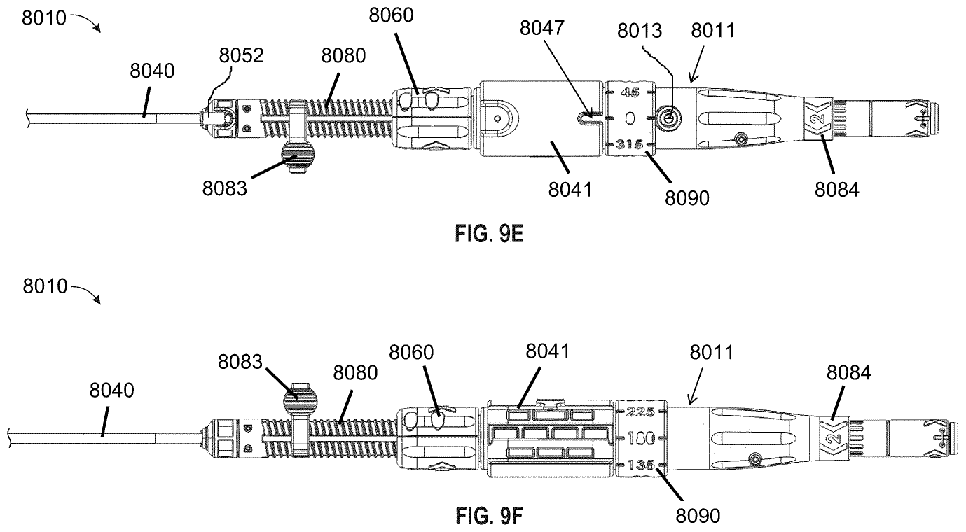

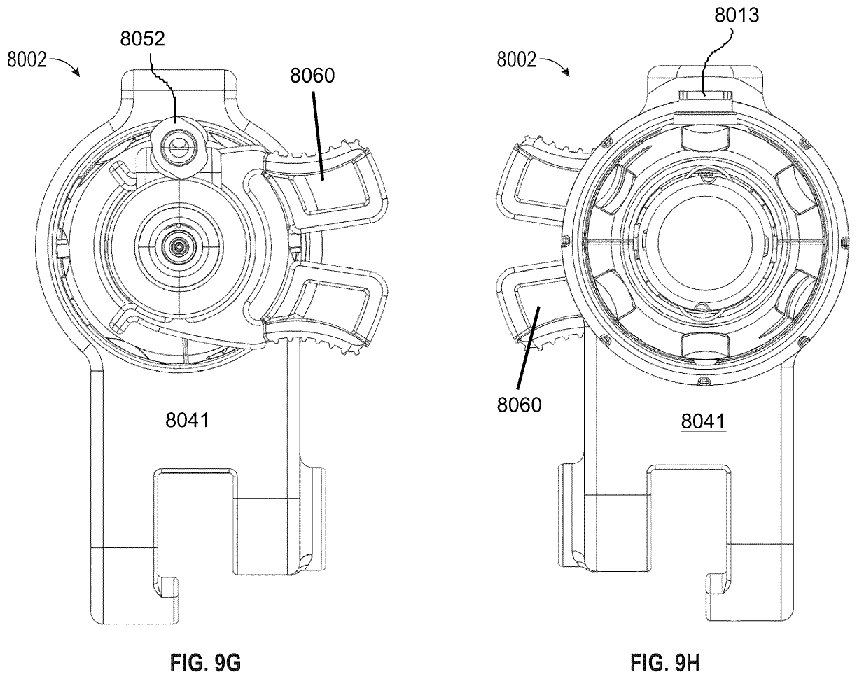

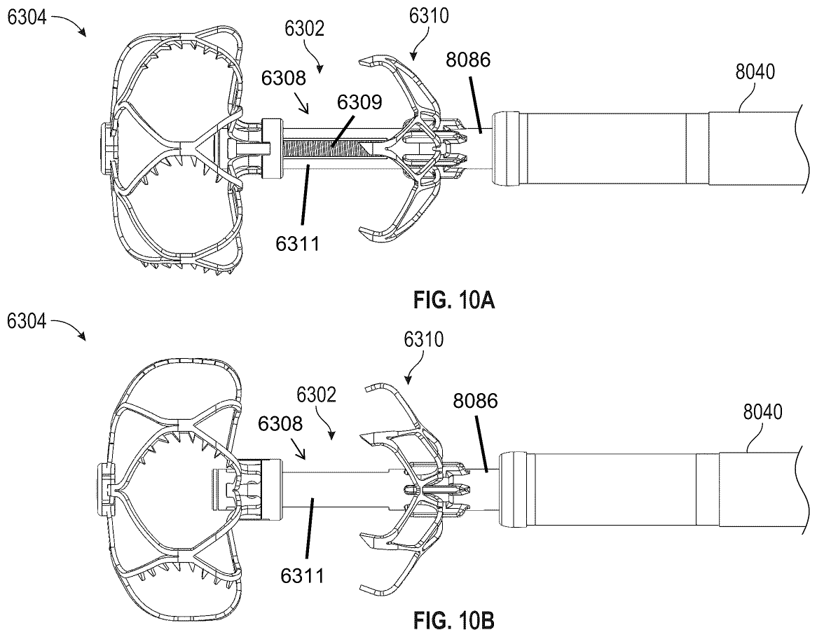

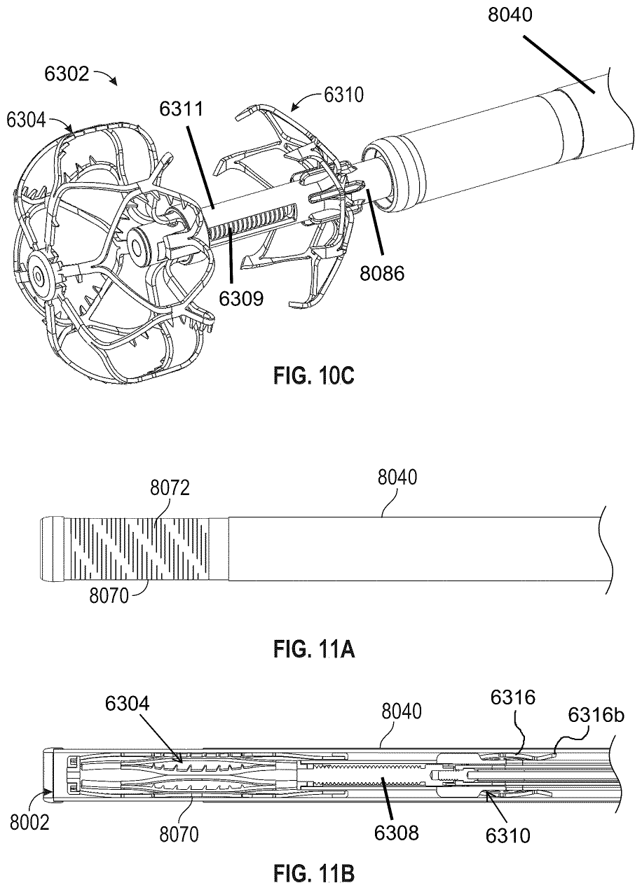

FIGS. 9A-9H shows a portion of an arrangement of delivery catheter 8010 that can be used with any arrangements of the implant disclosed herein. As described in greater detail below, and as shown in FIGS. 8A-8B, delivery catheter 8010 may be advanced to position in the patient via guide catheter 8014. FIGS. 10A-10C show a side view of the arrangement of the implant 6304 and distal end portion of delivery catheter 8010, showing the outer sheath 8040 partially withdrawn and the contact member 6304 and the securing element 6310 each in an expanded state.

With reference to FIG. 8A, the support stand 8012 can be positioned on a support surface, such as a bed or table, or can be positioned on a patient's body. In some arrangements, the support stand 8012 can have a low slip surface on the bottom thereof; or can be positioned on a low slip material or surface such as a low-slip mat. As will be discussed, the support stand 8012 can be used to removably support the delivery catheter 8010 (also referred to herein as a procedure catheter). Some arrangements of the support stand 8012 can include a base portion 8030, a first support portion 8032 having a slot 8033 therein, and a second support 8034 having a first locking element 8038 coupled therewith. Some arrangements of the support stand 8012 can have a scale used to measure or provide a measurement reference to a user of the system to determine how far delivery catheter 8010 is moved along the slot 8033. The scale can provide millimeter and/or centimeter markings. For example, and without limitation, this can assist the user in determining how far the implant is being moved relative to the user's anatomy during the procedure. In the present example, a clamp element 8041 is provided about a handle 8011 of delivery catheter 8010. Clamp element 8041 has a tab or projection 8043 extending therefrom that is sized and configured to extend into the slot 8033 to help maintain an alignment of the delivery catheter 8010 to the first support portion 8032.

The second support portion 8034 can be configured to support the guide catheter 8014 and dilator (not shown), and the first locking element 8038 can be selectively adjusted or tightened to secure a guide sheath 8015 of the guide catheter 8014 in a desired axial and/or rotational position after the guide sheath 8015 has been advanced to the target location using the steering device 8016. Using standard percutaneous techniques, a guidewire (e.g., a 0.035 in guidewire) and an introducer (e.g., a 6-8 Fr introducer) can be advanced into the patient's vasculature (e.g., the femoral vein, contralateral femoral artery, or a radial artery) and the guidewire and dilator can be advanced into the left atrium. An accepted or suitable transeptal procedure can be performed to achieve transeptal access.

The steerable guide catheter 8014 can be advanced over the guidewire and supported by or coupled with the second support 8034. In some arrangements, the metal tubing of the guide catheter 8014 can be secured to the second support 8034. A second locking element 8039 can be used to secure the delivery catheter 8010 in the desired position relative to the guide catheter 8014 and relative to the support stand 8012. With the dilator removed, a distal end of the outer sheath 8040 of the delivery catheter 8010 can be inserted into the guide sheath and advanced to the target location. FIG. 8 illustrates this system after the outer sheath 8040 of the delivery catheter 8010 has been advanced into the guide catheter 8014. Angiograms or other suitable imaging techniques can be performed at any step in the process for baseline purposes and for visualization during the procedure.

The outer sheath 8040 of the delivery catheter 8010 can be advanced and withdrawn axially relative to the guide sheath 8015 by sliding the delivery catheter 8010 distally and proximally, respectively, along the slot 8033 in the first support portion 8032 of the support stand 8012. By distally translating the delivery catheter 8010 toward the guide catheter 8014, the outer sheath 8040 of the delivery catheter 8010 can be advanced past a distal end of the guide sheath 8015. FIG. 8 shows the implant device 6302 after the implant device 6302 has been advanced past a distal end 8040b of the outer sheath 8040. As described, by proximally withdrawing the outer sheath 8040 relative to the implant device 6302, the contact member 6304 and/or the securing element 6310 of the implant device 6302 can self-expand, depending on how far the outer sheath 8040 is withdrawn relative to the implant device 6302. The outer sheath 8040 can be advanced or withdrawn by rotating a first dial 8060 of the delivery catheter 8010 in either a first direction (e.g., clockwise) or a second direction (e.g., counterclockwise), respectively. Alternatively, in any arrangements, the contact member 6304 and/or the securing element 6310 of the implant device 6302 can be advanced past a distal end 8040b of the outer sheath 8040 by distally advancing the implant device 6302 relative to the outer sheath 8040. FIG. 8 shows both the contact member 6304 and the securing element 6310 advanced past the distal end 8040b of the outer sheath 8040 and in an expanded state.

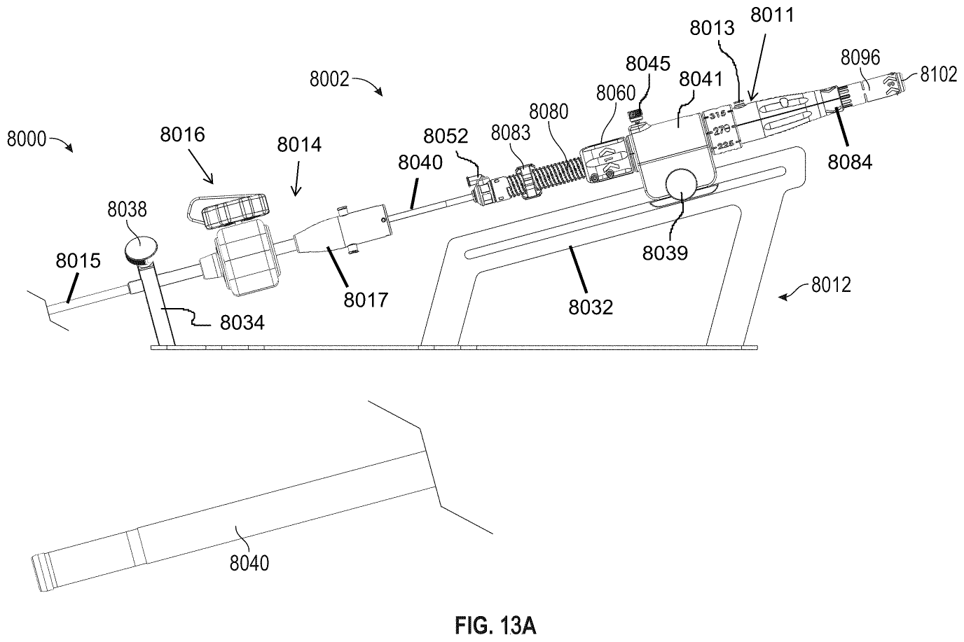

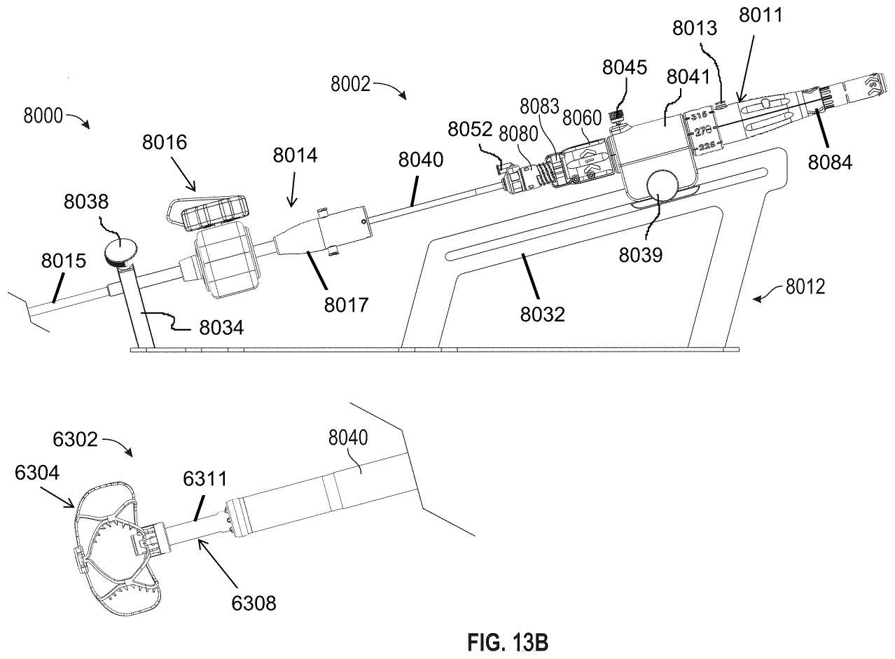

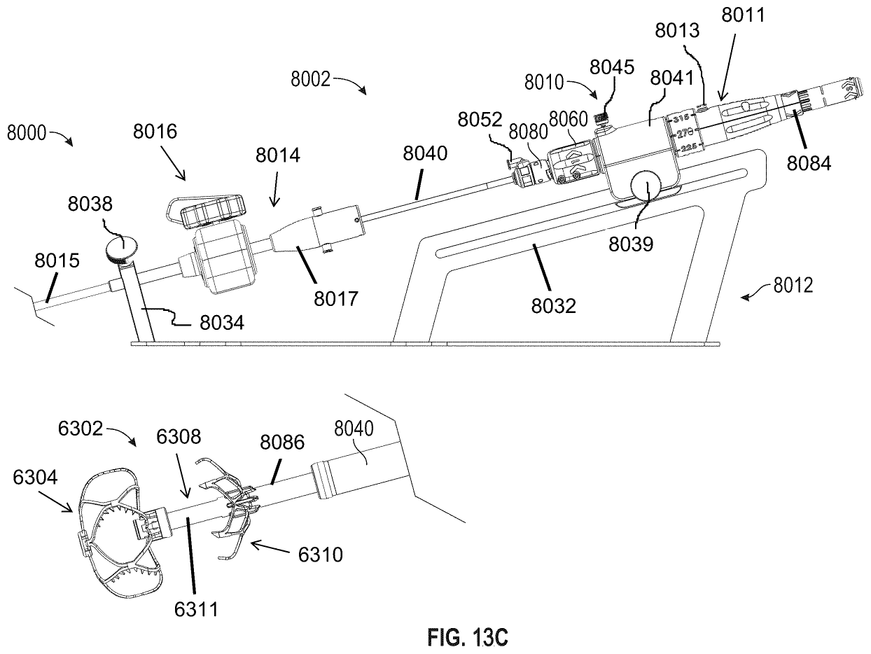

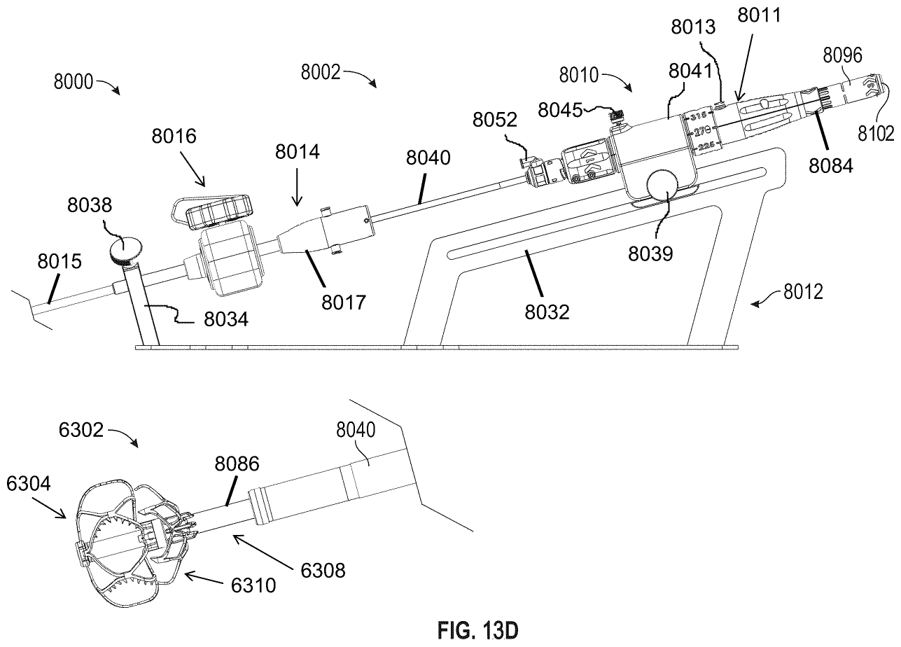

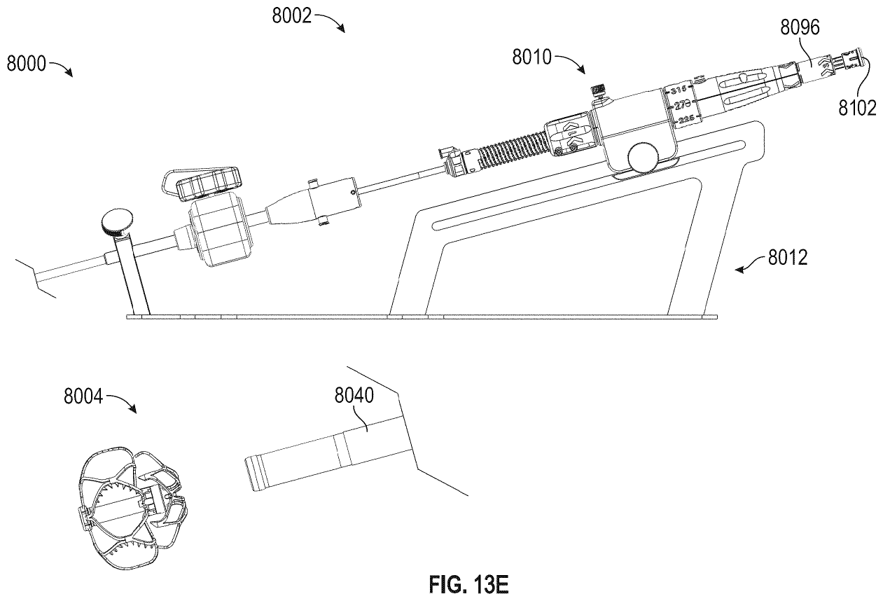

FIGS. 12A through 12D show structural relationships between various features in the proximal portion of delivery catheter 8010 and the distal portion of delivery catheter 8010. For instance, FIG. 12A shows the outer sheath 8040 and the first dial 8060 which is rotationally coupled with the outer sheath 8040, in solid black shading. In some arrangements, delivery catheter 8010 can be configured such that a rotation of the first dial 8060 in a first direction (e.g., clockwise) can cause a threaded connector 8080 that can be coupled with the outer sheath 8040 to thread into a threaded opening in the first dial 8060 (e.g., moving the threaded connector 8080 from a first position as shown in FIG. 13A to a second position as shown in FIG. 13B), thereby causing the outer sheath 8040 to move in the proximal direction. Similarly, delivery catheter 8010 can be configured such that a rotation of the first dial 8060 in a second direction (e.g., counterclockwise) can cause a threaded connector 8080 that can be coupled with the outer sheath 8040 to thread out of the opening in the first dial 8060, thereby causing the outer sheath 8040 to move in the distal direction. This movement of the outer sheath 8040 can result in the distal end 8040b of the outer sheath 8040 moving relative to the implant device 6302, to thereby unrestrain the implant device 6302.

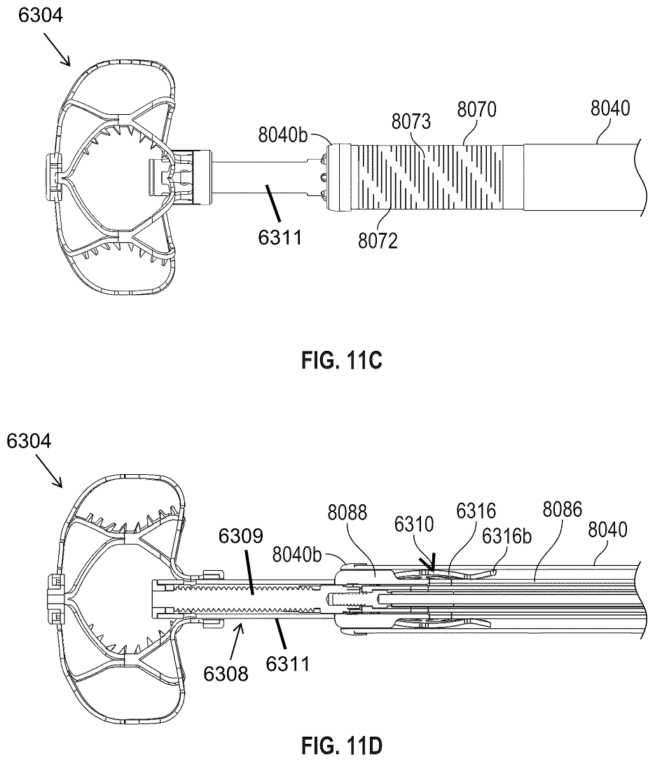

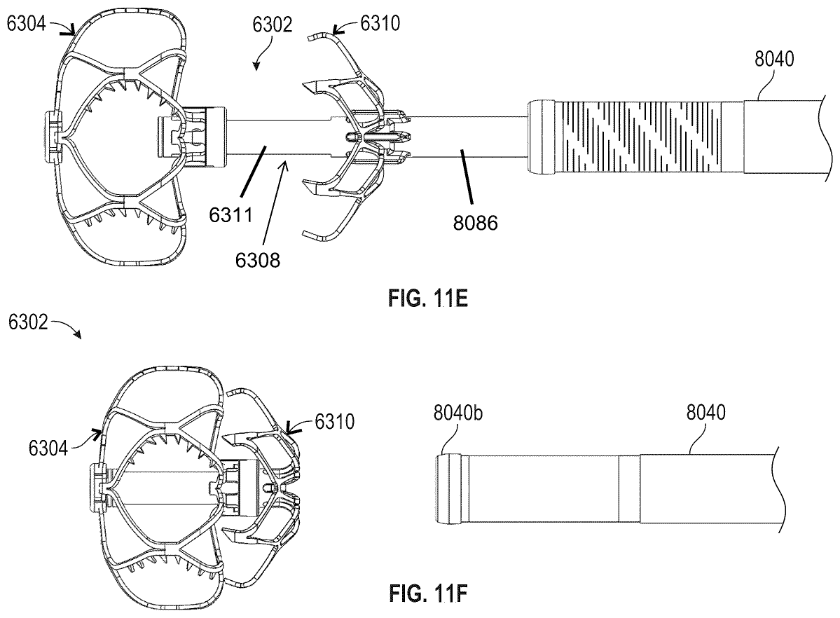

In any arrangements disclosed herein, delivery catheter 8010 can be configured to selectively limit an amount of rotation of the first dial 8060 relative to the threaded connector 8080. This can be done to inhibit (e.g., prevent) the user from inadvertently releasing the securing element 6310 from the outer sheath 8040 before the user is ready to. For example, and without limitation, some arrangements of delivery catheter 8010 can have a stop element (e.g., a clip) 8083 that can be removably coupled with the threaded connector 8080 at any desired position on the threaded connector 8080. The stop element 8083 can be coupled with the threaded connector 8080 at a position such that, when the stop element 8083 abuts a distal end of the first dial 8060, the first dial 8060 is inhibited (e.g., prevented) from rotating further in the direction that would cause the threaded connector 8080 to thread into the first dial 8060, thereby preventing further retraction of the outer sheath 8040 until the clip is moved distally or, more commonly, removed from the outer sheath 8040. In some arrangements, stop element 8083 positioned as shown in FIG. 9A can abut the distal end of the first dial 8060 when the outer sheath 8040 is positioned as shown in FIG. 11D (where the securing element 6310 is positioned completely or nearly completely within the outer sheath 8040). The stop element 8083 can be removed from the threaded connector 8080 to continue rotation of the first dial 8060 in the direction that causes further retraction of the outer sheath 8040, as shown in FIG. 13C.

FIG. 12B illustrates delivery catheter 8010 and the implant device 6302, showing an inner catheter 8086 (also referred to herein as a first inner catheter or inner catheter member) having one or a plurality of fins 8088 at a distal end 8086b of the inner catheter 8086 (also referred to herein as an inner tube), and a second dial 8084 which is rotationally coupled with the inner catheter 8086, in solid black shading. An axial translation in a first direction and/or a rotation of the second dial 8084 (along with the entire handle 8011 of delivery catheter 8010) in a first direction will cause a commensurate axial translation and/or rotation of the inner catheter 8086, respectively, which will cause a commensurate axial translation and/or rotation of the implant device 6302, respectively, when the implant device 6302 is engaged with the inner catheter 8086. In some arrangements, the inner catheter 8086 can have one or more or a plurality of fins 8088 extending in a radial direction at a distal end 8086 of the inner catheter 8086. The fins 8088 can be configured to fit between the struts 6316 of the securing element 6310 at least when the securing element 6310 is in a restrained position within the outer sheath 8040. In this arrangement, the user can rotate the contact member and/or the securing element by rotating the second dial 8084 and, hence, the inner catheter 8086.

In some arrangements, delivery catheter 8010 can have an angle gauge 8090 configured to rotate with the second dial 8084 so that a user can measure or track an angle of rotation of the second dial 8084. This can provide visual feedback to the user of generally how much the implant is being rotated during the procedure. In some arrangements, the angle gauge 8090 can be configured to be adjustable or selectively rotatable relative to the second dial 8084. For example, and without limitation, in some arrangements, the user can disengage the coupling mechanism that selectively rotationally couples the angle gauge 8090 to the second dial 8084 so that the user can rotate the angle gauge 8090 relative to the second dial 8084—e.g., to reset the angle gauge 8090 to a zero reading relative to an indicator on delivery catheter 8010. In some arrangements, the coupling mechanism can be a ball and detent or detents, or a plurality of balls and detents, that can be configured to bias the angle gauge 8090 to be rotationally coupled with the second dial 8084 but can be overcome by exerting a threshold torque on the angle gauge 8090 relative to the second dial 8084. The angle gauge 8090 can have readings at 45 degrees, or 30 degrees, or 15 degrees. The optional ball and detent mechanism can be configured to have a detent every 45 degrees, or 30 degrees, or 15 degrees that can be sized and configured to receive the ball so that the user can selectively rotate the angle gauge 8090 relative to the second dial 8084 and then recouple the angle gauge 8090 to the second dial 8084 at such increment (e.g., at 45 degrees, or 30 degrees, or 15 degrees). Other selective rotational coupling mechanisms can also be used to bias or selectively rotationally couple the angle gauge 8090 to the second dial 8084.

With reference to FIG. 12C, after the contact member 6304 has been rotated and/or translated as desired such that the tissue of the LAA and/or the left atrium has constricted around the contact member 6304, the user can then advance the securing element 6310 toward the contact member 6304 by rotating a linking element 8096 (also referred to herein as a sleeve) of delivery catheter 8010. The sleeve 8096 can be rotationally coupled with or otherwise keyed with a third dial 8097 (not shown in FIG. 12C) and a fourth dial 8102 with one or a plurality of splines, teeth, and/or other engagement features when the sleeve 8096 is in a first position (as shown in FIG. 12C). The third dial 8097 can be axially and rotationally coupled with a second inner catheter 8098 (also referred to herein as a second inner catheter member) positioned inside the inner catheter 8086. The sleeve 8096 and the second inner catheter 8098 are shown in solid black shading in FIG. 12C for reference.

A distal end 8098b of the second inner catheter 8098 can be rotationally coupled with the head 6330 that is coupled with the threaded shaft 6309 of the retention element 6308 when the distal end 8098b of the second inner catheter 8098 is engaged with the head 6330 such that, when the sleeve 8096 is in the first position, a rotation of the sleeve 8096 will cause the simultaneous and equal rotation of the third dial 8097 and the second inner catheter 8098 so as to rotate the retention element 6308. In this state, a rotation of the sleeve 8096 in a first direction can cause the retention element 6308 to rotate in a first direction and move the securing element 6310 toward the contact member 6304 (e.g., to a position as shown in FIG. 13D) and a rotation of the sleeve 8096 in a second, opposite direction can cause the retention element 6308 to rotate in a second direction and move the securing element 6310 away from the contact member 6304. By rotating the sleeve 8096 in the first direction, the user can advance the securing element 6310 toward the tissues of the LAA and/or left atrium that has been constricted by the rotation of the contact member 6304 to bias the tissue of the LAA and/or the left atrium in the closed or occluded state.

Some arrangements of the delivery catheter 8010 can have a fourth dial 8102 that can be used to release the implant from delivery catheter 8010, as shown in FIG. 13E. The fourth dial 8102 can be positioned at a proximal end of handle 8011 of delivery catheter 8010 and can be coupled with an inner core 8106 (also referred to herein as a third inner catheter member) that is positioned inside of the second inner catheter 8098. The fourth dial 8102 and the inner core 8106 are shown in solid black shading in FIG. 12D for reference. The inner core 8106 of this example has a threaded end portion 8108 having external threads thereon that can be threadedly engaged with internal threads of threaded opening 6315 at the proximal end of retention element 6308. The threaded end portion 8108 can be used to selectively couple the inner core 8106 with the retention element 6308. The threaded portion 8108 can be removed from the retention element 6308 by rotating the threaded portion 8108 relative to the retention element 6308 in a first direction (e.g., counter clockwise) that causes the threaded portion 8108 to unthread from the threaded opening 6315 at the proximal end of retention element 6308. In some arrangements, the retention element 6308 can be held in a fixed rotational position with the inner catheter 8086, e.g., while the fourth dial 8102 is turned to remove the inner core 8106 from the retention element 6308. Thus, in some arrangements, when the user is ready to release the implant from delivery catheter 8010, the user can unthread the threaded portion 8108 from threaded opening 6315 of retention element 6308 and then axially withdraw delivery catheter 8010, with the implant device 6302 remaining in the deployed position within the LAA/left atrium.

With reference to FIG. 12D, in some arrangements, the sleeve 8096 can be selectively axially coupled with the fourth dial 8102. The sleeve 8096, when axially coupled with the fourth dial 8102, can prevent the independent rotation of the fourth dial 8102 relative to other components of delivery catheter 8010, including without limitation the third dial 8097. In some arrangements, the sleeve 8096 can be rotationally coupled with a fourth dial 8102 with one or a plurality of splines, teeth, and/or other engagement features when the sleeve 8096 is in the first position (as shown in FIG. 12C)—e.g., when the sleeve 8096 is overlapping the fourth dial 8102 sufficient to engage the complementary splines, teeth, and/or other engagement features of the sleeve 8096 and the fourth dial 8102. When the sleeve 8096 is slid in an axial position so that the splines, teeth, and/or other engagement features of the sleeve 8096 are not engaged with the complementary splines, teeth, and/or other engagement features of the fourth dial 8102 (e.g., to a second position, as shown in FIG. 12D), the fourth dial 8102 can then be rotated in the first direction to remove the threaded portion 8108 from the retention element 6308, as discussed above.

In some arrangements, the sleeve 8096 can be selectively inhibited (e.g., selectively prevented) from moving axially from the first position of the sleeve 8096 to the second position of the sleeve 8096. For example, and without limitation, a removable locking element 8110 can be used to selectively inhibit the sleeve 8096 from moving axially from the first position to the second position. Locking element 8110 may also provide concomitant rotation of fourth dial 8102 with sleeve 8096, which may further provide concomitant rotation of inner core 8106 with second inner catheter 8098. In some arrangements, the removable locking element 8110 can be a suture, as shown in FIG. 8B that passes through one, two, or more openings 8112 in the sleeve 8096 that are aligned with one, two, or more openings in the fourth dial 8102 that are aligned with the one, two, or more openings 8112 in the sleeve 8096 when the sleeve 8096 is in the first position. In this state, the removable locking element 8110 prevents the axial movement of the sleeve 8096 relative to the fourth dial 8102. In this state, with the sleeve 8096 coupled with the fourth dial 8102, delivery catheter 8010 can therefore prevent the user from inadvertently releasing the implant from the delivery catheter. In some arrangements, the user can remove the removable locking element 8110 to permit the sleeve 8096 to be moved to the second position of the sleeve 8096 and to thereafter rotate the fourth dial 8102 to release the implant. For example, and without limitation, where the removable locking element 8110 is a suture, the user can cut and withdraw the suture from delivery catheter 8010, move the sleeve 8096 in the axial direction to the second position, and then rotate the fourth dial 8102 to release the implant from delivery catheter 8010.

FIGS. 11A-11D show an arrangement of a distal end portion of the outer sheath 8040. In any arrangements of the system, the outer sheath 8040 can be used to restrain the implant device 6302, including the contact member 6304 and the securing element 6310. Additionally, in any arrangements disclosed herein, the contact member 6304 and the securing element 6310 can be self-expanding such that when the contact member 6304 and/or the securing element 6310 are advanced past distal 8040b end of outer sheath 8040, the contact member 6304 and the securing element 6310 can each expand from a first, compressed state to a second, enlarged or expanded state. In some such versions, contact member 6304 and/or securing element 6310 are also temperature sensitive, such that contact member 6304 and/or securing element 6310 will not expand to the enlarged/expanded state until contact member 6304 and/or securing element 6310 exceed a certain temperature threshold. In some such cases, room temperature is below the temperature threshold; while the temperature of the body of the patient (e.g., approximately 98.6° F.) is above the temperature threshold. By way of further example only, the material providing such properties may include nitinol. Alternatively, any other suitable material(s) may be used to form contact member 6304 and/or securing element 6310; and any suitable temperature threshold may be provided to transition contact member 6304 and/or securing element 6310 from the first state to the second state. In still other variations, a temperature threshold is not needed to transition contact member 6304 and/or securing element 6310 from the first state to the second state.

In the initial configuration of some arrangements of the system 8000, to ensure that struts 6316 of the securing element 6310 can expand and ensure that distal end portions 6316b of the struts 6316 of the securing element 6310 are not within tissue that has constricted or collapsed around the implant device 6302 or the outer sheath 8040, the securing element 6310 can be arranged in the system so that distal end portions 6316b of struts 6316 are pointing in the proximal direction when the securing element 6310 is restrained within the outer sheath 8040 in the initial configuration. An example of this structural relationship and orientation of struts 6316 is shown in FIGS. 11B and 11D.

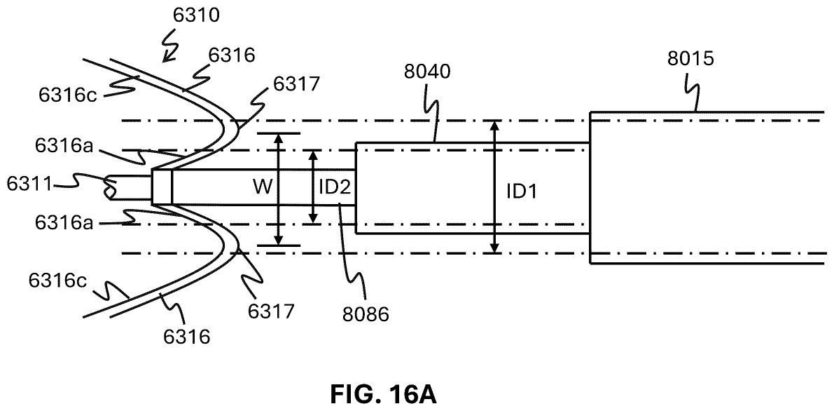

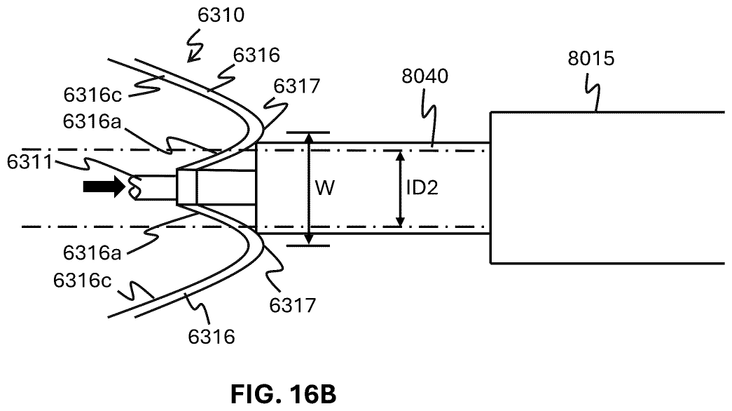

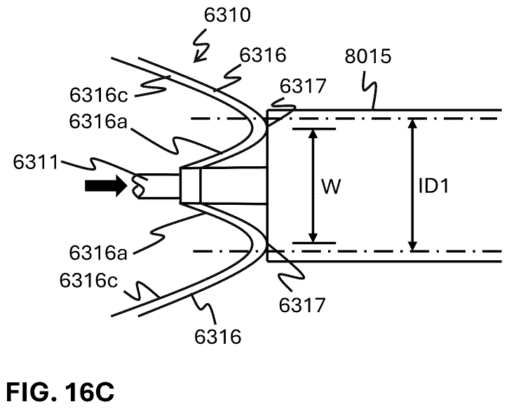

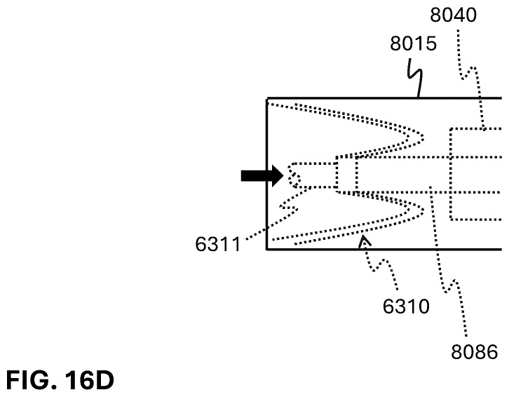

Any arrangements of the implant device 6302 can be configured such that, after the contact member 6304 and the securing element 6310 have been expanded to the second, expanded state, the contact member 6304 and/or the securing element 6310 can be collapsed or returned to the collapsed state within the outer sheath 8040 by distally advancing the outer sheath 8040. In this configuration, distally advancing the outer sheath 8040 can cause the securing element 6310 to collapse such that the distal end portions 6316b of the struts 6316 of the securing element 6310 are pointing in a distal direction. Further advancement of the outer sheath 8040 can also cause the distal end 8040b of the outer sheath 8040 to move against the contact member 6304 and cause the contact member 6304 to collapse to the initial collapsed state within the outer sheath. This can be beneficial for recapturing the implant or repositioning the contact member 6304 and/or the securing element 6310. In some variations, as described below with reference to FIGS. 16A-16D, outer sheath 8040 is not capable of transitioning securing element 6310 from a deployed, expanded state to a contained, compressed state. Instead, guide sheath 8015 is capable of transitioning securing element 6310 from a deployed, expanded state to a contained, compressed state.

In the present example, the distal end portion of the outer sheath 8040 has a distal section 8070 having a plurality of openings 8072 therein. In some arrangements, the openings 8072 can be laser cut into the distal section 8070. In some arrangements, the openings 8072 can be 0.001 inch in width, or approximately 0.001 inch in width. The openings 8072 can be configured to permit a fluid (e.g., a contrast medium) to flow therethrough when a pressure differential between the inside the outer sheath 8040 and the outside of the outer sheath 8040 reach a predetermined or threshold value. In the present example, the size and spacing of openings 8072 is selected to strike an appropriate balance. Specifically, in cases where openings 8072 are too large, there may be a risk of distal end portions 6316b of struts 6316 getting caught in openings 8072 during operation of delivery catheter 8010 and thereby snagging on outer sheath 8040. Moreover, openings 8072 may undesirably increase friction between implant device 6302 and distal section 8070 of outer sheath 8040 if openings 8072 are too large. On the other hand, if openings 8072 are too small, openings 8072 might not provide a desired flow of contrast medium through openings 8072. Thus, openings 8072 of the present example are large enough to provide the desired flow of contrast medium while avoiding the risk of snagging distal end portions 6316b of struts 6316 and undesirable friction between implant device 6302 and outer sheath 8040.

In the event that distal end 8040b of outer sheath 8040 is blocked by tissue when fluid (e.g., contrast medium) is injected into outer sheath 8040, or in the event that some other phenomenon is providing sufficient backpressure within outer sheath, openings 8072 may provide a vent or pressure relief pathway by allowing such fluid to leak out from outer sheath 8040. Similarly, openings 8072 can be positioned to permit a user to determine a location of the tissue of the LAA and/or the left atrium that has been constricted by the implant device 6302. For example, and without limitation, when the implant device 6302 has been rotated and the tissue of the LAA and/or the left atrium has been wrapped around a portion of the implant device 6302, it may be helpful for the physician to determine where such wrapped tissue is positioned or where an end portion of the tissue is on the implant device 6302. The tissue may be difficult or impossible to view in fluoroscopy. In this situation, contrast medium can be advanced through the outer sheath 8040 and through the openings 8072 to the extent that the openings 8072 are not covered by tissue. When the tissue covers at least some of the openings 8072, the tissue can block a passage of or inhibit a passage of a fluid (e.g., the contrast medium) through such openings 8072. Therefore, if contrast medium flows through at least some of the openings 8072, such flow may be observed to determine the extent to which openings 8072 are blocked by tissue, which may further indicate the length to which tissue is wrapped around distal section 8070 of outer sheath 8040. In some arrangements, the openings 8072 can in this manner be used to determine generally a position of the tissue relative to the distal end 8040b of the outer sheath 8040, which can also be configured to be visible in fluoroscopy.

In addition to the foregoing, a radiopaque band, coating, marker, or other feature(s) can be used to provide visibility of at least a portion of distal section 8070 of outer sheath 8040 in fluoroscopy. It should also be understood that openings 8072 may enhance the echogenicity of distal section 8070 of outer sheath 8040, thereby facilitating visualization of distal section 8070 under ultrasound imaging.

In some arrangements, the securing element 6310 can also be configured to be visible in fluoroscopy and therefore can be visualized by a user. Therefore, a user can use the openings 8072 to determine a position of the constricted tissue that surrounds the outer sheath 8040 relative to the securing element 6310 to determine if the securing element 6310 can be expanded and deployed. If the distal end portion 6316b of each of the struts 6316 of the securing element 6310 is distal to the constricted tissue that surrounds the outer sheath 8040 when the implant is axially positioned as shown in FIG. 11D (e.g., so that a distal end 8086b of the inner catheter 8086 is positioned adjacent or within a predetermined distance from a distal end 8040b of the outer sheath 8040), then the securing element 6310 is not in an optimal position for deployment by withdrawal of the outer sheath 8040. The user may release and reengage the LAA or otherwise adjust a position of the implant relative to the constricted tissue to ensure that at least the distal end portion 6316b of each of the struts 6316 of the securing element 6310 is distal to the constricted tissue that surrounds the outer sheath 8040 when the implant is axially positioned as shown in FIG. 11D.

In some arrangements, the openings 8072 may be a plurality of circumferentially extending slits that extend circumferentially around the distal section 8070. For example without limitation, each of the slits can have a length that is 10% or approximately 10% of a circumference of the distal section 8070, or from 10%, approximately 10%, or less than 10% to 50%, approximately 50%, or more than 50%, or from 15% or approximately 15% to 40% or approximately 40%, or from 15% or approximately 15% to 20% or approximately 20% of the circumference of the distal section 8070, or any value or range of values within any of the foregoing ranges. In some arrangements, the distal section 8070 can have a space 8073 between each of the openings 8072 in the circumferential direction. In some arrangements, the space 8073 between each of the openings 8072 can form a helical or angled pattern on the distal section 8070.

In any arrangements disclosed herein, any of the tubes, catheters, or sheaths (including, without limitation, the outer sheath 8040, the inner catheter 8086, and the second inner catheter 8098 described in greater detail below) may comprise metal, which may include stainless steel. For instance, some versions of outer sheath 8040 may have a polymeric exterior portion with a metallic lining inside the polymeric exterior portion. By way of further example only, such a metallic lining may be beneficial by promoting sliding engagement between outer sheath 8040 and implant device 6302 (which is metallic in the present example), where such sliding contact will not cause skiving, gouging, or other potentially undesirable effects on outer sheath 8040. Utilizing a metallic lining in sheath 8040 to promote sliding engagement between outer sheath 8040 and implant device 6302 may also mitigate risk of damage to implant device 6302 that might otherwise occur in the event that implant device 6302 would snag on non-metallic material in outer sheath 8040 while outer sheath 8040 slides relative to implant device 6302 (or while implant device 6302 slides relative to outer sheath 8040), particularly to the extent that implant device 6302 is resiliently biased to expand outwardly and thereby bear against the inner sidewall of outer sheath 8040.

In any arrangements, the tubes, catheters, or sheaths can be laser cut, which can improve flexibility of the tube, catheter, or sheath. Some arrangements of the outer sheath 8040 can include a laser cut, stainless steel tube and a polymer (e.g., Pebax) jacket over at least a portion thereof. For example, and without limitation, for the outer sheath 8040, a polymer jacket can extend from a proximal end of the outer sheath 8040 to a proximal end of the distal section 8070 so as to not cover the openings 8072 in the distal section 8070. In some arrangements, the openings 8072 can extend along an entire length or substantially an entire length of the metal layer of the outer sheath 8040 and the polymer jacket can extend from a proximal end of the outer sheath 8040 to a proximal end of the distal section 8070 so as to not cover the openings 8072 in the distal section 8070. In some arrangements, a distal end portion of the jacket can be heat melted to the metal tube. For this purpose, so that the jacket does not flow through the openings 8072, a band or section without openings can be formed that can be aligned with the distal end of the jacket.

In any arrangements of the system 8000, a size, pattern, and angular orientation of the openings can vary along a length of the metal tube to provide variations in the flexibility of the tube, catheter, or sheath. For example, an angle of the spaces 8073 between the openings 8072 relative to a longitudinal centerline axis of the sheath can be greater at a distal end of the sheath to provide greater flexibility at the distal end thereof. A proximal end portion of the sheath can be designed to be stiffer, such as by angling the spaces between the openings at a lower angle relative to the longitudinal axis of the sheath, and a medium stiffness portion can extend between the proximal and distal portions.

In some arrangements, the delivery catheter 8010 can have one or more biasing elements (e.g., spring or springs) to bias some of the components of the delivery catheter 8010 to move relative to one another or to exert a force on one or more of the components of the delivery catheter 8010. For example, and without limitation, in some arrangements, one or more springs or other biasing element(s) can be used to pull the fourth dial 8102 and the inner core 8106 in a proximal direction relative to the second inner catheter 8098. This can bias the second inner catheter 8098 to remain in contact and engagement with the retention element 6308 and/or the head 6330 of the retention element 6308. Additionally, in some arrangements, one or more springs or other biasing element(s) can be used to pull the fourth dial 8102, the inner core 8106, and/or the second inner catheter 8098 in a proximal direction relative to the inner catheter 8086. This can bias the inner catheter 8086 to remain in contact and engagement with the securing element 6310. To the extent that one or more springs or other biasing element(s) are used to maintain positive engagement between components of delivery catheter 8010, as described above or otherwise, this maintaining of positive engagement may mitigate manufacturing tolerances of and between such components.

In addition, as described in greater detail below with respect to the release of implant device 6302 from delivery catheter 8010, one or more springs or other biasing element(s) may cause inner core 8106 and fourth dial 8102 to suddenly move proximally through a certain range of longitudinal motion, which may provide visual indication to the physician that implant device 6302 has been released from delivery catheter 8010. In addition, by urging inner core 8106 to travel through a certain range of proximal motion upon release of implant device 6302 from delivery catheter 8010, one or more springs or other biasing element(s) may be sufficient to cause threaded portion 8108 to be positioned proximally relative to distal end 8040b of outer sheath 8040 upon release of implant device 6302 from delivery catheter 8010. In such versions, threaded portion 8108 will not protrude distally past distal end 8040b of outer sheath 8040 after implant device 6302 is fully released from delivery catheter 8010. This may in turn prevent threaded portion 8108 from snagging on tissue or instrument components during removal of delivery catheter 8010 from the patient after delivery of implant device 6302.

### IV. Example of Method of Using LAA Treatment Device Delivery System

### A. Example of Installing Implant in the Patient

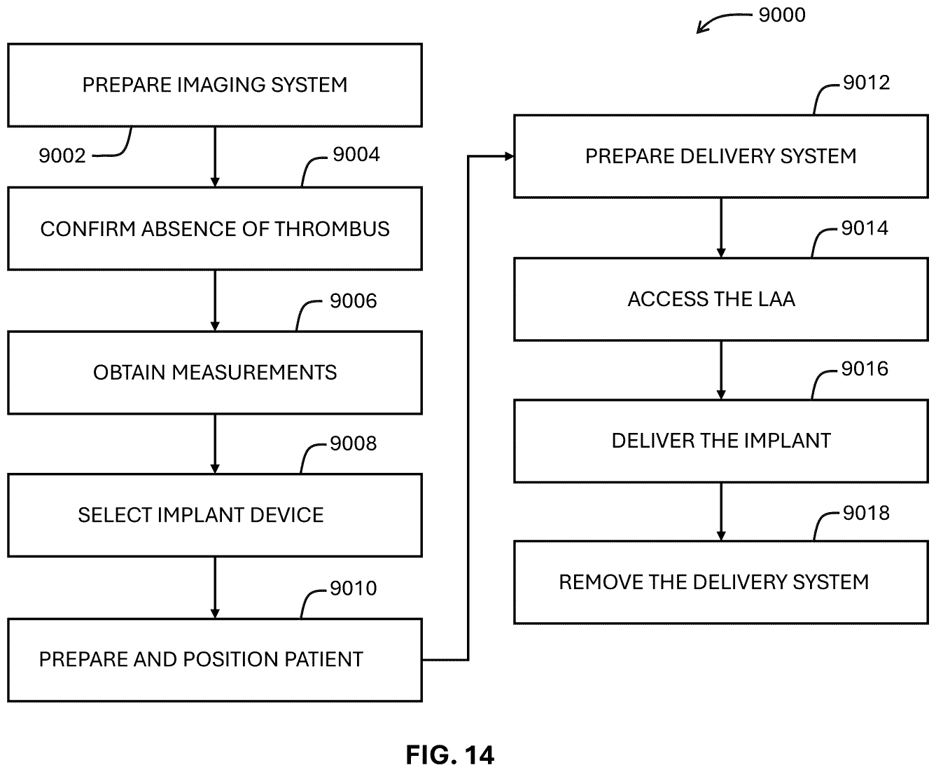

As noted above, FIGS. 13A through 13D show the system 8000 at various stages of use according to certain examples. The following description provides another example of a use case in which system 8000 may be employed. The use case of the present example is illustrated as a method 9000 in FIG. 14. As an initial matter, the physician may first prepare components of one or more imaging systems (block 9002), such as a fluoroscopy imaging system, a transesophageal echocardiogram (TEE) imaging system, and/or any other suitable kind(s) of imaging system(s). The physician may also confirm the absence of an intracardiac thrombus (block 9004). The physician may then measure (block 9006) the width of the LAA ostium and the contact depth of the LAA of the patient (e.g., via TEE, etc.). By way of example only, the LAA ostium width may be measured along the axis between the left circumflex artery (LCx), on the mitral side, and approximately 1.5 centimeters down from the tip of the limbus. By way of further example only, the LAA contact depth may be measured perpendicularly from the center of the LAA ostium diameter line to the back of the LAA in the 0°, 90°, and 135° views. However, in the 45° view, the LAA contact depth may be measured from the aortic valve edge (left-most side) of the LAA ostium to the back of the LAA at an angle of 45° (upper left to lower right). Alternatively, the width of the LAA ostium and the contact depth of the LAA may be measured in any other suitable fashion. The physician may further assess the shape of the LAA, the number of lobes of the LAA, and the location of lobes relative to the LAA ostium.

The physician may then select (block 9008) an appropriately sized implant device 6302 from various sized implant devices 6302, based at least in part on the patient-specific LAA data described above. As noted above, in some cases, the size of implant device 6302 may vary based on the outer width W of the contact member 6304. For instance, an array of implant devices 6302 may include contact members 6304 having outer widths W of approximately 10 mm, approximately 12 mm, approximately 16 mm, and approximately 18 mm. As also noted above, in some cases, the size of the securing element 6310 may be the same among the array of implant devices 6302 with contact members 6304 having different respective outer widths W, such that the size of the securing element 6310 need not necessarily vary with the size of the contact member 6304. However, in some other cases, the lengths of struts 6316 of the securing elements 6310 may vary based on the outer width W of contact member 6304.

By way of example only, a physician may select an implant device 6302 having a contact member 6304 with an outer width W of approximately 10 mm in cases where the LAA ostium diameter ranges from approximately 11 mm to approximately 14 mm, with an LAA contact depth ranging from approximately 10 mm to approximately 16 mm. A physician may select an implant device 6302 having a contact member 6304 with an outer width W of approximately 12 mm in cases where the LAA ostium diameter ranges from approximately 13 mm to approximately 19 mm, with an LAA contact depth ranging from approximately 12 mm to approximately 18 mm. A physician may select an implant device 6302 having a contact member 6304 with an outer width W of approximately 16 mm in cases where the LAA ostium diameter ranges from approximately 18 mm to approximately 24 mm, with an LAA contact depth ranging from approximately 14 mm to approximately 20 mm. A physician may select an implant device 6302 having a contact member 6304 with an outer width W of approximately 18 mm in cases where the LAA ostium diameter ranges from approximately 23 mm to approximately 26 mm, with an LAA contact depth ranging from approximately 16 mm to approximately 20 mm. These parameters are merely illustrative examples, and different contact members 6304 with different outer widths W may be selected based on any other suitable criteria.

With the appropriate implant device 6302 selected, the patient may be prepared (block 9010) for transseptal catheterization and positioned relative to the support stand 8012. In some cases, the patient's leg is placed on a lift (not shown), with the support stand 8012 also being placed on the lift. In some such cases, the lift may be oriented at an angle such that a proximal portion of the lift is lower than a distal portion of the lift, such that the patient's leg is supported at a corresponding angle.

Once the patient has been sufficiently prepared, delivery system 8000 may then be prepared (block 9012) for use by first removing delivery catheter 8010, guide catheter 8014, and a dilator (not shown) from their sterile packaging. After these components are removed from their sterile packaging, a basin or other container may be filled with a volume (e.g., 500 cc) of heparinized saline. A syringe (e.g., a 66 cc syringe) may be coupled with the dilator and used to de-air the dilator with heparinized saline. The dilator may then be set aside with the syringe still attached, and with some saline remaining in the syringe. Fluid conduit 8022 may be coupled with luer port 8019 of hub 8017 of guide catheter 8014, with fitting 8024 being secured to the opposite end of fluid conduit 8022. Fitting 8024 may also be coupled directly with the other luer port 8019 of hub 8017 of guide catheter 8014. With hub 8017 being oriented vertically, the chamber between hemostasis valves within hub 8017 may be de-aired with heparinized saline via a syringe. An empty syringe may then be coupled with an open port of fitting 8024.

To de-air guide catheter 8014, the distal tip of guide sheath 8015 may be submerged in the basin of heparinized saline. While the distal tip of guide sheath 8015 of guide catheter 8014 submerged in the basin of heparinized saline, hub 8017 of guide catheter 8014 may be raised at an angle (e.g., approximately 30 degrees) up from the preparation table. While tapping hub 8017 and the length of guide sheath 8015, a volume (e.g., 60 cc) of the heparinized saline may be aspirated with the syringe, thereby de-airing hub 8017 of guide catheter 8014. A valve of fitting 8024 may then be closed and the syringe may be removed. Hub 8017 may then be raised to a vertical position and hub 8017 may be checked for leaks for a desired duration (e.g., 30 seconds). In any air is seen entering hub 8017, guide catheter 8014 may be rejected and another guide catheter 8014 prepared. If no air is seen entering hub 8017, hub 8017 may be lowered down to the preparation table. A syringe containing heparinized saline may then be connected to fitting 8024. Fitting 8024 may then be de-aired and opened.

While the dilator is being flushed with heparinized saline, the dilator may be advanced into the hemostasis valves of hub 8017 until the tip of the dilator enters guide sheath 8015 of guide catheter 8014. The flushing of dilator may then cease, leaving a syringe attached to the dilator, and the dilator may continue to be advanced until a certain visual indicator (e.g., black band) on the shaft of the dilator is aligned with the proximal end of hub 8017. The distal tip of guide sheath 8015 may then be removed from the basin of heparinized saline, and the syringe may be removed from the dilator. Fitting 8024 may then be transitioned to a closed state and the syringe may be removed from hub 8017. Next, the distal end of guide sheath 8015 may be resubmerged in the basin of heparinized saline and the deflection of guide sheath 8015 may be tested by turning steering knob 8020 (e.g., 90 degrees clockwise) and returning to the neutral position. If this test is passed, the combination of guide catheter 8014 and the dilator may be removed from the tub of heparinized saline and set aside.

Delivery catheter 8010 may then be removed from its tray, and locking elements 8038, 8039, 8045 are removed from their tray. A threaded shaft of locking element 8045 is inserted into a corresponding opening at the top of clamp element 8041 and rotated until the threaded shaft of locking element 8045 seats against a body of handle 8011, without being fully tightened. A threaded shaft of locking element 8039 is inserted into a corresponding opening at the side of clamp element 8041 until the threading is flush with clamp element 8041. A fluid conduit 8050 (see FIG. 8B) is then coupled with a luer port 8052 at the distal end of handle 8011. A three-way stopcock (not shown) is then coupled with the other end of fluid conduit 8050. A syringe containing heparinized saline is further to proximal luer 8013 of handle 8011, and delivery catheter 8010 is then flushed with the heparinized saline while tapping handle 8011 and the length of outer sheath 8040 until the heparinized saline exits the open 3-way stopcock attached to the fluid conduit 8050. The three-way stopcock may then be closed while the flushing of delivery catheter 8010 continues, along with the tapping of handle 8011 and the length of outer sheath 8040.

The syringe may then be removed from proximal luer 8013. A syringe containing heparinized saline may then be coupled with the 3-way stopcock, which may be opened to allow flushing of delivery catheter 8010 while tapping handle 8011 and the length of outer sheath 8040. With saline left in the syringe, distal end 8040b of outer sheath 8040 may be plugged (e.g., with a finger, etc.) to ensure flow of fluid through openings 8072. The three-way stopcock may then be closed. The syringe may then be refilled and reconnected to the three-way stopcock. The three-way stopcock may then be re-opened. The “0 degrees” indicator on angle gauge 8090 may then be aligned with proximal luer 8013 and a corresponding notch 8047 (see FIGS. 9A-9B and 9E) atop clamp element 8041. Locking element 8045 may then be tightened to secure the angular position of handle 8011 relative to clamp element 8041.

In some versions, a stop clip (not shown) is secured to threaded connector 8080, at the distal end of first dial 8060, before delivery catheter 8010 is placed in its packaging for shipment. In such versions, this stop clip may be removed at this stage. Delivery catheter 8010 may then be set aside, with distal end 8040b of outer sheath 8040 slightly elevated. Support stand 8012 may then be removed from its packaging and placed on the sterile preparation table. Support stand 8012 may be inspected for corrosion. A threaded shaft of locking element 8038 may be inserted into support 8034 until the threading is flush with support 8034. The steps described above for preparing delivery system 8000 for use are just examples. Other methods may be used to prepare delivery system 8000 for use.

With delivery system 8000 thus prepared, delivery system 8000 may be used to access the LAA (block 9014). For instance, conventional percutaneous techniques may be employed to achieve femoral access. In cases where a baseline coronary angiography is performed, contralateral femoral arterial access or radial arterial access may be obtained using conventional techniques. A transseptal puncture may be performed using any suitable transseptal puncture instrumentation and any suitable conventional techniques under any suitable imaging guidance (e.g., fluoroscopy and echo). In some cases, the transseptal puncture is oriented inferior to posterior. Once transseptal puncture is achieved, the therapeutic activated clotting time may be ensured to be longer than a certain duration (e.g., longer than 250 seconds). A guidewire (not shown) of any suitable size (e.g., approximately 0.035 inches) may be advanced into the left upper pulmonary vein or looped within the left atrium. Angiographic views of the LAA may then be obtained.

Next, the combination of the dilator and guide catheter 8014 may be advanced along the guidewire into the left atrium. To the extent that the underside of support stand 8012 does not include an integral anti-slip surface, a sterile anti-slip pad may be placed over the lift on which the patient's leg is placed. Support stand 8012 may then be placed on the anti-slip pad, such that support stand 8012 and the patient's leg are both supported by support stand 8012. Guide catheter 8014 may then be inserted through a slot of support 8034, and locking element 8038 may be actuated to secure guide catheter 8014 relative to support 8034. The tip of the guidewire may then be retracted into the tip of the dilator, and then the combination of the guidewire and dilator may be removed from guide catheter 8014 while gentle aspiration is provided via guide catheter 8014. In some cases, the physician may use imaging guidance to ensure that the distal tip of guide sheath 8015 is not positioned against the wall of the left atrium or other tissue during aspiration.

After the combination of the guidewire and dilator have been removed from guide catheter 8014, delivery catheter 8010 may be transferred from the device preparation table to the operating table. During such transit, distal end 8040b of outer sheath 8040 and handle 8011 may be maintained at a similar height to avoid introduction of air into delivery catheter 8010. In addition, delivery catheter 8010 may be continually flushed with an attached syringe (e.g., at a trip rate of 1 to 2 drops per second) during this transfer. It may be acceptable for outer sheath 8040 to have a slight bend during this transfer. Delivery catheter 8010 may then be advanced into guide catheter 8014 using a fluid-to-fluid technique. The flushing of delivery catheter 8010 may cease once distal end 8040b of the outer sheath 8040 is sufficiently disposed in hub 8017 of guide catheter 8014. In some cases, hub 8017 may be positioned at the same vertical level as the patient during this process to allow blood of the patient to fill the lumen of guide catheter 8014.

The physician may continue to advance delivery catheter 8010 into guide catheter 8014 until a certain visual indicator (e.g., black band) on outer sheath 8040 of delivery catheter 8010 is flush with the proximal end of hub 8014. Clamp element 8041 may then be secured loosely to support portion 8032 of support stand 8012. Under imaging guidance (e.g., fluoroscopy), distal end 8040b of outer sheath 8040 may be advanced at least a certain distance (e.g., at least 2 cm) beyond the distal end of guide sheath 8015. At this stage, the position of distal end 8040b of outer sheath 8040 may be adjusted as needed through a process of iterative adjustments. Such iterative adjustments may include adjustments of the position of the distal end of guide sheath 8015 (e.g., via steering device 8016). Such iterative adjustments may also include rotation of guide sheath 8015 (e.g., via steering device 8016). Such iterative adjustments may also include translation of handle 8011 along support portion 8032 to position distal end 8040b of outer sheath 8040 within the center of the LAA ostium. Once distal end 8040b of outer sheath 8040 has been suitably positioned in the center of the LAA ostium, all three locking elements 8038, 8039, 8045 may be tightened to thereby fix the positions of guide sheath 8015 and outer sheath 8040 relative to the patient. At this stage, the components of delivery system 8000 may appear as shown in FIGS. 8B and 13A.

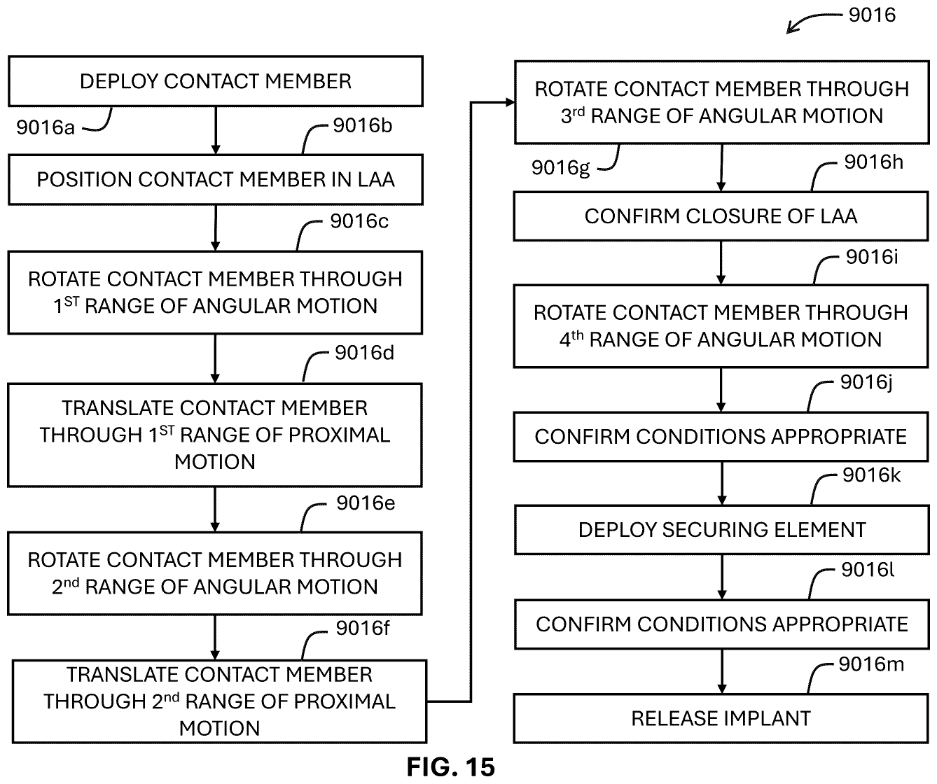

Once the LAA has been suitably accessed, with distal end 8040b of outer sheath 8040 being suitably positioned in the center of the LAA ostium, the implant device 6302 may be delivered (block 9016). Examples of steps of the implant delivery process 9016 are shown in FIG. 15. As a first step of the implant delivery process, contact member 6304 may be deployed (block 9016a) relative to outer sheath 8040. To deploy contact member 6304, under imaging guidance (e.g., fluoroscopy and/or TEE, etc.), the physician may rotate first dial 8060 clockwise until contact member 6304 and body portion 6311 are fully deployed and first dial 8060 contacts stop element 8083. The resulting arrangement may appear similar to what is shown in FIGS. 11C-11D and 13B. As described above with reference to FIG. 12A, first dial 8060 and threaded connector 8080 cooperate to provide longitudinal movement of outer sheath 8040, such that rotation of first dial 8060 clockwise provides proximal retraction of outer sheath 8040 to thereby uncover contact member 6304 and body portion 6311. In other words, contact member 6304 and body portion 6311 remain longitudinally stationary while outer sheath 8040 retracts proximally in response to rotation of first dial 8060. Nevertheless, in some cases, the entire delivery catheter 8010 may be advanced distally while outer sheath 8040 is being retracted proximally, to thereby ensure that contact member 6304 does not deploy within guide sheath 8015 of guide catheter 8014.

In some cases, first dial 8060 may contact stop element 8083 before body portion 6311 is fully deployed from guide sheath 8015. In such scenarios, stop element 8083 may be removed from threaded connector 8080, and the physician may continue to slowly rotate first dial 8060 until body portion 6311 is fully deployed from guide sheath 8015. At this stage, stop element 8083 may be again secured to threaded connector 8080, against the distal face of first dial 8060, to restrict further longitudinal movement of threaded connector 8080 (and, hence, outer sheath 8040) relative to first dial 8060. During the stage where stop element 8083 is removed from threaded connector 8080, it may be appropriate for the physician to ensure that securing element 6310 is not prematurely deployed from guide sheath 8015.

Once contact member 6304 and body portion 6311 have been suitably deployed from guide sheath 8015, first dial 8060 may be rotated counterclockwise to a neutral position. This may prevent further deployment of implant device 6302 during subsequent rotation and translation steps. The position of contact member 6304 may be further adjusted under imaging guidance (e.g., fluoroscopy and/or TEE, etc.) through a process of iterative adjustments. Such iterative adjustments may include adjustments of the position of the distal end of guide sheath 8015 (e.g., via steering device 8016). Such iterative adjustments may also include rotation of guide sheath 8015 (e.g., via steering device 8016). Such iterative adjustments may also include translation of handle 8011 along support portion 8032 to position contact member 6304 within the center of the LAA ostium and orthogonal to the plane of the LAA ostium.

Next, locking element 8039 may be loosened to allow repositioning of delivery catheter 8010 along support portion 8032 to thereby position contact member 6304 in the LAA (block 9016b). Under imaging guidance (e.g., fluoroscopy and/or TEE, etc.), the physician may advance delivery catheter 8010 to position contact member 6304 into the LAA until contact member 6304 makes contact with the inner wall of the LAA. In some cases, at this stage of operation, the position of contact member 6304 within the LAA may appear similar to what is shown in FIG. 2H. When contact member 6304 makes sufficient contact with the inner wall of the LAA, locking element 8039 may be tightened to secure the position of delivery catheter 8010 along support portion 8032. The sufficiency of the contact between contact member 6304 and the inner wall of the LAA may again be confirmed with one or more imaging modalities (e.g., fluoroscopy and/or TEE, etc.). At this stage, in cases where TEE imaging is used, the TEE imaging may be set to a three-dimensional en-face view or a three-dimensional multiplanar reconstruction view. Angle gauge 8090 may be rotated to indicate a zero-degree angular position, aligning with notch 8047 of clamp element 8041.

Next, contact member 6304 may be rotated within the LAA through a first range of angular motion (block 9016c). To accomplish this, locking element 8045 may be loosened to allow rotation of delivery catheter 8010 relative to clamp element 8041. Under imaging guidance (e.g., fluoroscopy and/or TEE, etc.), the physician may rotate second dial 8084 counterclockwise. This clockwise rotation of second dial 8084 may rotate contact member 6304 via inner catheter 8086 as described above with reference to FIG. 12B. As contact member 6304 rotates within the LAA, and due to the tangential orientation aspect of tissue anchors 6319 as described above, tissue anchors 6319 engage the wall of the LAA, such that rotation of contact member 6304 causes the LAA to twist/wrap as described above with reference to FIGS. 2C, 2J, and 7. In the event that tissue anchors 6319 are not sufficiently engaged with the wall of the LAA to cause twisting/wrapping of the LAA when contact member 6304 is rotated within the LAA, contact member 6304 may be repositioned. In such scenarios, contact member 6304 may first be retracted proximally from the LAA, then repositioned while contact member 6304 is within the left atrium, then be re-advanced into the LAA after being repositioned within the contact member 6304. In other words, the repositioning of contact member 6304 may occur within the left atrium (and not in contact with any anatomical structures) rather than occurring within the LAA.

In cases where tissue anchors 6319 are sufficiently engaged with the wall of the LAA to cause twisting/wrapping of the LAA when contact member 6304 is rotated within the LAA, second dial 8084 may be rotated counterclockwise through the first range of angular motion (e.g., an angular range of anywhere from approximately 180 degrees to approximately 360 degrees; or more specifically, approximately 270 degrees), and then locking element 8045 may be tightened to secure the angular position of second dial 8084 (and, hence, contact member 6304). The range of angular motion of second dial 8084 (and, hence, contact member 6304) may be tracked through visual observation of angle gauge 8090 in relation to notch 8047 of clamp element 8041. Locking element 8045 may then be tightened after the first range of angular motion has been completed, to thereby secure the angular position of contact member 6304.

With the first range of angular motion complete, contact member 6304 may be translated through a first range of proximal motion (block 9016d). To accomplish this, locking element 8039 may be loosened to allow longitudinal movement of delivery catheter 8010 along first support portion 8032 of support stand 8012. Delivery catheter 8010 (and, hence, contact member 6304) may then be retracted proximally through the first range of proximal motion (e.g., a range of anywhere from approximately 1 mm to approximately 15 mm; from approximately 1 mm to approximately 6 mm; or more specifically, from approximately 2 mm to approximately 3 mm). As contact member 6304 is retracted proximally, and due to the proximal orientation aspect of tissue anchors 6319 as described above, tissue anchors 6319 engage the wall of the LAA, such that proximal retraction of contact member 6304 causes the LAA to compress longitudinally. After such proximal retraction, a contrast medium may be injected via openings 8072 of outer sheath 8040 as described above to ensure that contact member 6304 continues to engage the LAA. In some cases, some of the contrast medium may still enter the LAA even if contact member 6304 sufficiently engages the LAA at this stage. Locking element 8039 may again be tightened to secure the longitudinal position of delivery catheter 8010 (and, hence, contact member 6304) relative to first support portion 8032 of support stand 8012.

After completing the first range of proximal motion, contact member 6304 may be rotated through a second range of angular motion (block 9016c). To accomplish this, second dial 8084 may be grasped with a first hand; and locking element 8045 may be loosened with a second hand while the first hand grasps second dial 8084. Second dial 8084 may then be rotated to drive counterclockwise through the second range of angular motion (e.g., an angular range of anywhere from approximately 15 degrees to approximately 90 degrees; or more specifically, approximately 45 degrees). This may cause further twisting/wrapping of the LAA due to engagement of the tissue anchors 6319 with the wall of the LAA. Locking element 8045 may then be tightened after the second range of angular motion has been completed, to thereby secure the angular position of contact member 6304.

With the second range of angular motion complete, contact member 6304 may be translated through a second range of proximal motion (block 9016f). To accomplish this, locking element 8039 may be loosened to again allow longitudinal movement of delivery catheter 8010 along first support portion 8032 of support stand 8012. Delivery catheter 8010 (and, hence, contact member 6304) may then be retracted proximally through the second range of proximal motion (e.g., a range of anywhere from approximately 1 mm to approximately 15 mm; from approximately 1 mm to approximately 6 mm; or more specifically, from approximately 2 mm to approximately 3 mm). This may cause further longitudinal compression of the LAA due to engagement of the tissue anchors 6319 with the wall of the LAA. After completing the second range of proximal motion, locking element 8039 may again be tightened to secure the longitudinal position of delivery catheter 8010 (and, hence, contact member 6304) relative to first support portion 8032 of support stand 8012.

After completing the second range of proximal motion, contact member 6304 may be rotated through a third range of angular motion (block 9016g). To accomplish this, second dial 8084 may be grasped with a first hand; and locking element 8045 may be loosened with a second hand while the first hand grasps second dial 8084. Second dial 8084 may then be rotated to drive counterclockwise through the third range of angular motion (e.g., an angular range of anywhere from approximately 15 degrees to approximately 90 degrees; or more specifically, approximately 45 degrees). This may cause further twisting/wrapping of the LAA due to engagement of the tissue anchors 6319 with the wall of the LAA. Locking element 8045 may then be tightened after the second range of angular motion has been completed, to thereby secure the angular position of contact member 6304.

After completing the third range of angular motion, the physician may confirm closure of the LAA (bock 9016h). To accomplish this, contrast medium may be injected via openings 8072 of outer sheath 8040 to ensure sufficient twisting/wrapping of the LAA. For instance, if any contrast medium enters the LAA at this stage, such entry of contrast medium into the LAA may indicate that the LAA has not yet been sufficiently twisted/wrapped, as sufficient twisting/wrapping of the LAA would result in no fluids entering the LAA from the left atrium at this stage. In addition to using contrast medium, one or more other imaging modalities (e.g., TEE) may be used to evaluate whether the LAA has been sufficiently twisted/wrapped by contact member 6304 at this stage. In the event that contrast medium testing and/or imaging reveals that the LAA has not been sufficiently twisted/wrapped by contact member 6304 at this stage, at least some of the steps described above may be repeated until contrast medium testing and/or imaging reveals that the LAA has been sufficiently twisted/wrapped by contact member 6304 (e.g., such that no contrast medium or other fluid enters the twisted/wrapped LAA).

In the event that the LAA needs to be untwisted/unwrapped, locking element 8045 may be loosened, and second dial 8084 may be rotated clockwise until angle gauge 8090 indicates zero-degree positioning in relation to notch 8047 of clamp element 8041. At this stage, contrast medium may be injected via openings 8072 of outer sheath 8040 to verify patency of the LAA. Contact member 6304 may then be retracted proximally out of the LAA and then repositioned within the left atrium, as described above. The physician may then re-advance contact member 6304 into the LAA and repeat the above-described sequence of rotation, retraction, etc.

In the event that the physician confirms that contact member 6304 has sufficiently twisted/wrapped the LAA, contact member 6304 may be rotated through a fourth range of angular motion (block 9016i). To accomplish this, second dial 8084 may be grasped with a first hand; and locking element 8045 may be loosened with a second hand while the first hand grasps second dial 8084. Second dial 8084 may then be rotated to drive counterclockwise through the fourth range of angular motion (e.g., an angular range of anywhere from approximately 15 degrees to approximately 90 degrees; or more specifically, approximately 45 degrees). This may cause further twisting/wrapping of the LAA due to engagement of the tissue anchors 6319 with the wall of the LAA. Locking element 8045 may then be tightened after the second range of angular motion has been completed, to thereby secure the angular position of contact member 6304. Even though the LAA may be sufficiently closed before contact member 6304 is rotated through the fourth range of angular motion, this additional rotation may further ensure that the LAA remains closed for an appropriate period of time after the implantation process is complete. As noted above with reference to FIG. 7, the sequence of rotating contact member 6304 through several ranges of angular motion and retracting contact member 6304 through several ranges of proximal motion may promote sufficient, lasting closure of the LAA, eliminating a risk of leak channels being formed by helical patterns 6368 in the twisted LAA tissue through a combination of torsional compression and longitudinal compression.

In some cases, contact member 6304 and/or a portion of delivery catheter 8010 may buckle or otherwise deform while contact member 6304 is rotated through the first, second, third, or fourth range of angular motion. Such deformation may occur before any tissue of the LAA or other anatomical structure is damaged, such that contact member 6304 may be configured to deform without damaging tissue of the LAA in the event that contact member 6304 is rotated to a point at which such deformation occurs. By way of example only, contact member 6304 may begin to deform when torsional forces on contact member 6304 exceed approximately 70 N-mm (10 in-ozf). Alternatively, contact member 6304 may begin to deform when torsional forces on contact member 6304 exceed any other suitable threshold. In the event that contact member 6304 is rotated to the point where contact member 6304 deforms, it may be appropriate to reverse at least some of the foregoing steps, reposition the approach of contact member 6304 into the LAA, and/or reposition the location of contact member 6304 into the LAA.

The physician may wish to further confirm that the deployment of contact member 6304 in the LAA, that the closure of the LAA is acceptable at this stage, and that conditions are otherwise appropriate for moving forward with the implantation process (block 9016j). For instance, the physician may check the coronaries for patency by performing angiography after contact member 6304 has been rotated through the fourth range of angular motion and held in place. The physician may also utilize imaging (e.g., fluoroscopy) to evaluate whether contact member 6304 is sufficiently symmetrical, with little to no deformation. The physician may also use TEE to confirm that the LAA is sufficiently closed. In addition, or in the alternative, the physician may use angiography to confirm that the LAA is sufficiently closed. In some such cases, the physician may inject contrast medium via openings 8072 of outer sheath 8040 and use angiography to observe whether such contrast medium enters the LAA. Similarly, the physician may rely on angiography and injection of contrast medium via openings 8072 to verify that tissue is not wrapped around distal end 8040b of outer sheath 8040. If the physician attempts to drive contrast medium out through openings 8072 and such contrast medium is not observed via angiography, this may indicate that tissue is undesirably wrapped around distal end 8040b of outer sheath 8040. If the physician determines that tissue is undesirably wrapped around distal end 8040b of outer sheath 8040, the physician may reverse at least some of the foregoing steps, reposition the approach of contact member 6304 into the LAA, and/or reposition the location of contact member 6304 into the LAA.

After confirming that the conditions are appropriate for moving forward with the implantation process, securing element 6310 may be deployed (block 9016k). To accomplish this, stop element 8083 may be removed from threaded connector 8080 and set aside. Under imaging guidance (e.g., fluoroscopy and/or TEE), first dial 8060 may be rotated clockwise to deploy securing element 6310. Specifically, rotation of first dial 8060 may provide proximal retraction of outer sheath 8040 via threaded connector 8080 as described above with reference to FIG. 12A. The resulting arrangement may appear similar to what is shown in FIGS. 8A, 10A-10C, 11E, 12A, and 13C. Imaging (e.g., fluoroscopy and/or TEE) may be used to visually confirm that all struts 6316 of securing element 6310 are fully deployed.

Next, sleeve 8096 may be rotated clockwise to advance securing element 6310 distally toward contact member 6304 until securing element 6310 and/or sleeve 8096 reaches the end of its travel. As described above with reference to FIG. 12C, rotation of sleeve 8096 causes rotation of second inner catheter 8098. As also described above, rotation of second inner catheter 8098 causes rotation of retention element 6308, which drives securing element 6310 toward contact member 6304. The resulting arrangement may appear similar to what is shown in FIG. 13D. At this stage of operation, distal end portions 6316b of struts 6316, and adjacent regions of struts 6316, should engage tissue (e.g., of the left atrium wall adjacent to the LAA ostium). During and after this stage of operation, the physician may wish to utilize imaging (e.g., fluoroscopy and/or TEE) confirm that none of the struts 6316 are bent rearwardly past a predetermined threshold angle (e.g., approximately 135 degrees) relative to the central longitudinal axis of body portion 6311 of securing element 6310. If the rearward bend angle of any strut 6316 exceeds the threshold value, the physician may rotate sleeve 8096 counterclockwise to retract securing element 6310 proximally away from contact member 6304 until none of the struts 6316 are bent rearwardly past the predetermined threshold angle. Alternatively, the physician may reverse at least some of the foregoing steps, reposition the approach of contact member 6304 into the LAA, and/or reposition the location of contact member 6304 into the LAA.

After confirming that none of the struts 6316 are bent rearwardly past the predetermined threshold angle, first dial 8060 may be rotated counterclockwise, to thereby advance outer sheath 8040 distally via threaded member 8080, until distal end 8040b of outer sheath 8040 is close to securing element 6310. As with other steps described herein, this step may be performed under imaging guidance (e.g., fluoroscopy and/or TEE). Locking element 8045 may then be loosened to evaluate the stability of implant device 6302 by allowing second dial 8084 to rotate freely in its neutral state. If stability of implant device 6302 is confirmed, locking element 8045 may be re-tightened.

At this stage, the physician may again confirm that various implantation conditions are satisfactory (block 90161). For instance, the physician may utilize imaging (e.g., fluoroscopy) to evaluate whether contact member 6304 is sufficiently symmetrical, with little to no deformation. The physician may also use TEE to confirm that the LAA is sufficiently closed. In addition, or in the alternative, the physician may use angiography to confirm that the LAA is sufficiently closed. In some such cases, the physician may inject contrast medium via openings 8072 of outer sheath 8040 and use angiography to observe whether such contrast medium enters the LAA. The physician may also use imaging (e.g., fluoroscopy and/or TEE) to visually confirm that securing element 6310 is sufficiently uniform and that the alignment or orientation of body portion 6311 is suitable. Confirming that securing element 6310 is sufficiently uniform may include confirming that securing element 6310 is suitable symmetric and/or that securing element 6310 engages tissue (of the left atrium wall adjacent to the LAA ostium) in a sufficiently uniform manner.

After confirming that the conditions are appropriate for moving forward with the implantation process, implant device 6302 may be released from delivery catheter 8010 (block 9016m). As noted above, a removable locking element 8110 such as a suture may be used to couple sleeve 8096 with fourth dial 8102; and locking element 8110 may prevent axial movement of sleeve 8096 relative to fourth dial 8102. Locking element 8110 may also provide concomitant rotation of fourth dial 8102 with sleeve 8096. After the physician has confirmed that various implantation conditions are satisfactory, the physician may remove locking element 8110. For instance, in versions where locking element 8110 comprises a suture, the suture may be cut and disposed of.

In some scenarios, use of a suture to provide locking element 8110 may provide some benefits. One such benefit may include the cutting of the suture having little to no impact on the position of delivery catheter 8010 to enable the release of implant device 6302 from delivery catheter 8110. Another benefit of using a suture to provide locking element 8110 may include making it difficult to re-lock the position of fourth dial 8102 relative to sleeve 8096. In other words, once the physician removes locking element 8110, the removal of locking element 8110 represents a commitment to complete the procedure. This may force the physician to take extra care to ensure that all the conditions are appropriate for moving forward with the procedure before locking element 8110 is removed. Yet another benefit of using a suture to provide locking element 8110 may include making it difficult to inadvertently unlock the position of fourth dial 8102 relative to sleeve 8096, such that the unlocking must be deliberate. While locking element 8110 is in the form of a suture in this example, locking element 8110 may alternatively take any other suitable form.

After locking element 8110 is removed, sleeve 8096 may be advanced distally until sleeve 8096 engages second dial 8084, exposing fourth dial 8102. The resulting arrangement of sleeve 8096 and fourth dial 8102 may appear similar to what is shown in FIG. 12D, though it should be understood that FIG. 12D also shows securing element 6310 in a proximal position relative to contact member 6304 when securing element 6310 would in fact be positioned further distally toward securing element 6310 in the sequence of operation presently described.

With fourth dial 8102 exposed, the physician may grasp sleeve 8096 with one hand and rotate fourth dial 8102 with the other hand while holding sleeve 8096 stationary. By way of example only, fourth dial 8102 may be rotated counterclockwise through approximately 6 revolutions. This counterclockwise rotation of fourth dial 8102 provides corresponding counterclockwise rotation of inner core 8106 as described above. As also described above, retention element 6308 is held in a fixed rotational position with the inner catheter 8086 while fourth dial 8102 and inner core 8106 are rotated. Thus, as inner core 8106 rotates counterclockwise, threaded portion 8108 of inner core 8106 unthreads from retention element 6308 of implant device 6302.

As further described above, some versions of delivery catheter 8010 may include a biasing element that resiliently urges fourth dial 8102 and inner core 8106 proximally. In some such versions, when inner core 8106 has been rotated counterclockwise sufficiently to disengage threaded portion 8108 from retention element 6308, the biasing element may cause inner core 8106 and fourth dial 8102 to suddenly move proximally through a certain range of longitudinal motion. This sudden proximal movement may provide visual confirmation to the physician that inner core 8106 has been rotated counterclockwise sufficiently to disengage threaded portion 8108 from retention element 6308. In addition, the range of proximal travel of inner core 8106 may be sufficient to cause threaded portion 8108 to be positioned proximally relative to distal end 8040b of outer sheath 8040. In such versions, threaded portion 8108 will not protrude distally past distal end 8040b of outer sheath 8040 after implant device 6302 is fully released from delivery catheter 8010. This may in turn prevent threaded portion 8108 from snagging on tissue or instrument components during removal of delivery catheter 8010 from the patient as described in greater detail below.

After inner core 8106 has been rotated counterclockwise sufficiently to disengage threaded portion 8108 from retention element 6308 (e.g., as indicated by the sudden proximal movement of inner core 8106 and fourth dial 8102), first dial 8060 may be rotated in the counterclockwise direction to drive outer sheath 8040 distally via threaded connector 8080, which may cause outer sheath 8040 to gently push implant device 6302 distally away from delivery catheter 8010. Locking element 8039 may then be loosened, and delivery catheter 8010 may then be moved proximally along first support portion 8032 of support stand 8012. The deployed implant device 6302 may come to its neutral state. The physician may again use imaging (e.g., TEE, etc.) to verify that the TEE remains fully closed.

After the implant device 6302 has been delivered and the physician has verified that the TEE remains fully closed, delivery system 8000 may be removed from the patient (block 9018). To accomplish this, with minimal steering on guide catheter 8014, guide catheter 8014 and delivery catheter 8010 may be removed together from the patient. Alternatively, delivery catheter 8010 may be removed first; and then guide catheter 8014 may be removed after delivery catheter 8010 has been removed. If delivery catheter 8010 and guide catheter 8014 are removed in a sequence, aspiration may be provided to guide catheter 8014 (e.g., via a luer port 8019 of hub) while delivery catheter 8010 is being removed from guide catheter 8014. After delivery catheter 8010 is removed from guide catheter 8104, guide catheter 8014 may be removed from the patient. Regardless of whether delivery catheter 8010 and guide catheter 8104 are removed from the patient simultaneously or in a sequence, the access site (e.g., incision through which guide sheath 8015 was inserted) may be closed using any suitable technique. This may mark the end of the method 9000 represented in FIG. 14, though it should be understood that various other steps may be added to method 9000 at any suitable stage(s).

While the method 9000 described above includes use of implant device 6302 to achieve closure of an LAA and maintain closure of an LAA, variations of the method 9000 may include use of implant device 6302 to implant device 6302 to achieve closure or occlusion of any other anatomical cavity or passageway, maintain closure or occlusion of the anatomical cavity, vessel, or other passageway; reshape the anatomical cavity, vessel, or other passageway; or otherwise modify the anatomical cavity, vessel, or other passageway. By way of example only, variations of the method 9000 may include use of implant device 6302 in a surface wound; an internal wound; a chamber, valve or cavity in the heart, lungs, stomach, or other organ; a gastrointestinal chamber or cavity; a blood vessel or other vessel; or other chamber or enclosure within the body (which can be the body of any living creature); and/or any other anatomical structure as may be apparent to those skilled in the art in view of the teachings herein. For shorthand purposes, the term “anatomical cavity” shall be understood to include all the anatomical cavities, vessels, or other passageways explicitly referenced above; as well as any other anatomical cavities, vessels, or other passageways that may be apparent to those skilled in the art in view of the teachings herein.

B. Example of Removal of Contact Member from the Patient

In some cases, it may be desirable to remove contact member 6304 from the patient after contact member 6304 has been deployed from outer sheath 8040 and rotated within the LAA; but before securing element 6310 has been deployed from outer sheath 8040. In some such scenarios, a process of removing contact member 6304 from the patient after contact member 6304 been deployed from outer sheath 8040 may be performed under image guidance (e.g., fluoroscopy and/or TEE) to monitor the location of contact member 6304 relative to the walls of the left atrium as contact member 6304 is recaptured within outer sheath 8040. Such a recapture process may begin with loosening locking element 8045 and then untwisting/unwrapping the LAA by rotating contact member 6304 counterclockwise via second dial 8084. The full untwisting/unwrapping of the LAA may be confirmed with fluoroscopy (e.g., including injection of contrast medium via guide catheter 8015), TEE, and/or other imaging.

After the full untwisting/unwrapping of the LAA is confirmed, contact member 6304 may be positioned centrally within the LAA, with minimal steering of guide catheter 8015. Then, all three locking elements 8038, 8039, 8045 may be tightened, and first dial 8060 may be rotated counterclockwise to advance outer sheath 8040 distally via threaded connector 8080. As outer sheath 8040 advances distally, distal end 8040b of outer sheath 8040 engages contact member 6304 and causes contact member 6304 to collapse. Further distal advancement of outer sheath 8040 eventually results in contact member 6304 being positioned proximally relative to distal end 8040b, proximal to distal end 8040b. At this stage, contact member 6304 may be fully recaptured within outer sheath 8040. Then, with minimal steering of guide catheter 8015, aspiration may be provided to guide catheter 8014 (e.g., via a luer port 8019 of hub) while delivery catheter 8010 may be removed from guide catheter 8014. At this stage, another delivery catheter 8010 with another implant device 6302 may be advanced into the patient via guide catheter 8014. Alternatively, guide catheter 8014 may be removed from the patient at this stage.

C. Example of Removal of Entire Implant from the Patient

In some cases, it may be desirable to remove implant device 6302 from the patient after contact member 6304 and securing element 6310 have been deployed from outer sheath 8040, after securing element 6310 has been rotated within the LAA, and after securing element 6310 has been advanced distally to engage tissue of the left atrium adjacent to the LAA ostium; but before implant device 6302 has been released from delivery catheter 8010. In some such scenarios, a process of removing implant device 6302 from the patient may be performed under image guidance (e.g., fluoroscopy and/or TEE) to monitor the location of contact member 6304 relative to the walls of the left atrium as contact member 6304 is recaptured within outer sheath 8040. An implant device 6302 removal process may begin with rotation of second dial 8084 counterclockwise until securing element 6310 is fully retracted proximally relative to contact member 6304 and the tissue of the left atrium adjacent to the LAA. Proximal retraction of securing element 6310 may cease before securing element 6310 is retracted into outer sheath. In other words, proximal retraction of securing element 6310 may cease while securing element 6310 is still distal to distal end 8040b of outer sheath 8040.

Next, locking element 8045 may loosened then the LAA may be untwisted/unwrapped by rotating contact member 6304 counterclockwise via second dial 8084. The full untwisting/unwrapping of the LAA may be confirmed with fluoroscopy (e.g., including injection of contrast medium via guide catheter 8015), TEE, and/or other imaging. After the full untwisting/unwrapping of the LAA is confirmed, contact member 6304 may be positioned centrally within the LAA, with minimal steering of guide catheter 8015. Then, first dial 8060 may be rotated in the counterclockwise direction to drive outer sheath 8040 distally via threaded connector 8080 until distal end 8040b of outer sheath 8040 contacts securing element 6310. After distal end 8040b of outer sheath 8040 contacts securing element 6310, first dial 8060 may be rotated further counterclockwise an additional full turn.

At this stage, locking element 8038 may be tightened; or its tightness confirmed. Locking element 8039 may be removed and set aside. With minimal steering of steerable guide 8014, delivery catheter 8010 may be retracted proximally until securing element 6310 is captured within guide sheath 8015. As part of this capture process, the distal end of guide sheath 8015 may engage securing element 6310 and cause securing element 6310 to collapse, such that securing element 6310 is fully captured within guide sheath 8015 in a collapsed state. Delivery catheter 8010 may be further retracted proximally until contact member 6304 is captured within guide sheath 8015. As part of this capture process, the distal end of guide sheath 8015 may engage contact member 6304 and cause contact member 6304 to collapse, such that contact member 6304 is fully captured within guide sheath 8015 in a collapsed state. At this stage, implant device 6302 may be fully recaptured within guide sheath 8015. Then, guide catheter 8015 and delivery catheter 8010 may be removed from the patient simultaneously.

In the present example, securing element 6310 and outer sheath 8040 are structurally configured such that securing element 6310 cannot be readily retracted proximally into outer sheath 8040 after securing element 6310 has been advanced distally out of outer sheath 8040 and transitions to an expanded configuration. In some versions where securing element 6310 cannot be readily retracted proximally into outer sheath 8040 after securing element 6310 has been advanced distally out of outer sheath 8040 and transitions to an expanded configuration, this may be due in part to properties of securing element 6310 representing a compromise that avoids high strains in struts 6316 and plastic deformation of struts 6316 that might otherwise occur if securing element 6310 were to be retractable proximally into outer sheath 8040 after securing element 6310 has been advanced distally out of outer sheath 8040 and transitions to an expanded configuration. There may nevertheless be versions where securing element 6310 can be readily retracted proximally into outer sheath 8040 after securing element 6310 has been advanced distally out of outer sheath 8040 and transitions to an expanded configuration, without such retraction resulting in plastic deformation of struts 6316 or other potentially undesirable results.

Even in versions where securing element 6310 cannot be readily retracted proximally into outer sheath 8040 after securing element 6310 has been advanced distally out of outer sheath 8040 and transitions to an expanded configuration, securing element 6310 may still be proximally retracted into guide sheath 8015 for removal from the patient as described above. An example of this process is shown in FIGS. 16A-16D. In particular, as shown in FIG. 16A, which only shows schematic representations of guide sheath 8015, outer sheath 8040, inner catheter 8086, securing element 6310, and body portion 6311, guide sheath 8015 has a first inner diameter ID1. Outer sheath 8040 has a second inner diameter ID2. Each strut 6316 of securing element 6310 has a bend apex 6317 that is interposed between proximal end portion 6316a and middle section 6316c. The bend apexes 6317 together define a width W.

The width W defined by bend apexes 6317 is larger the inner diameter ID2 of outer sheath 8040 but smaller than the inner diameter ID1 of guide sheath 8015 in the present example. Thus, if a physician were to attempt to retract securing element 6310 proximally into guide sheath 8040, proximal end portions 6316a of struts 6316 would snag on distal end 8040b of guide sheath 8040, with bend apexes 6317 being positioned outside the inner diameter ID2 of guide sheath 8040, as shown in the transition from the state shown in FIG. 16A to the state shown in FIG. 16B. However, when the entire delivery catheter 8010 and implant device 6302 are retracted proximally relative to guide sheath 8015, the larger inner diameter ID1 of guide sheath 8015 accommodates proximal end portions 6316a of struts 6316, the width defined by bend apexes 6317, and the rest of securing element 6310. This is shown in the transition from the state shown in FIG. 16A to the state shown in FIG. 16C, and further to the state shown in FIG. 16D. By way of further example only, the inner diameter ID1 of guide sheath 8015 may be approximately 18 French or any other suitable value.

In some arrangements, one or more or all of the components of the implant device and/or delivery device can be provided in a sterile condition. For example, and without limitation, one or more or all of the components of the implant device and/or delivery device can be provided in a sterile container or pouch, and can be double pouched. In some arrangements, some of the components of the delivery system can be provided non-sterile, and can be intended to be positioned and used on the non-sterile side of a sterile barrier in the operating room.

While securing element 6310 and outer sheath 8040 are structurally configured such that securing element 6310 cannot be readily retracted proximally into outer sheath 8040 after securing element 6310 has been advanced distally out of outer sheath 8040 and transitions to an expanded configuration in the present example, there may be other variations where securing element 6310 and outer sheath 8040 are structurally configured such that securing element 6310 can be readily retracted proximally into outer sheath 8040 after securing element 6310 has been advanced distally out of outer sheath 8040 and transitions to an expanded configuration.

The description of implant device 6302 provided above includes descriptions of various materials that may be used to form implant device 6302, as well as various dimensions (e.g., lengths, widths, etc.) and other structural details that may be incorporated into implant device 6302. While the materials and structural details as described above are merely illustrative examples and could be varied in different versions, in some cases there may be advantages to the examples of materials and structural details as described above, particularly when implant device 6302 is utilized in a procedure as described above. For instance, a contact member 6304 having the material and structural details as described herein may provide sufficient strength to rotate a pressurized LAA into a closed/wrapped state; while still having sufficient flexibility to be held within delivery catheter 8010, to be deployed from delivery catheter 8010 with a reasonable amount of force, and to be recaptured within delivery catheter 8010 if needed. Similarly, a contact member 6304 having the material and structural details as described herein may fit within delivery catheter 8010 with a reasonable amount of force such that contact member 6304 does not unduly tax the wall structures of delivery catheter 8010 while contact member 6304 is retained within delivery catheter 8010.

Similarly, tissue anchors 6118 (and other features of contact member 6304) having the material and structural details as described herein may provide sufficient grip to hold tissue of the LAA while not being so aggressive as to cause punctures to thin tissue. In some cases, this balance may be struck by virtue of tissue anchors 6118 being oriented circumferentially rather than radially outwardly.

By way of further example, securing element 6310 having the material and structural details as described herein may provide sufficient strength to suitably clamp tissue between securing element 6310 and contact member 6304, maintaining a sufficient clamping force for a sufficient amount of time. Conversely, securing element 6310 having the material and structural details as described herein may have sufficient flexibility to be held within delivery catheter 8010, to be deployed from delivery catheter 8010 with a reasonable amount of force, and to be recaptured within delivery catheter 8010 if needed. Conversely, securing element 6310 having the material and structural details as described herein may further provide sufficient torsional rigidity to counteract forces of the LAA to unwrap after the procedure described above is completed. Similarly, securing element 6310 having the material and structural details as described herein may provide substantial grip against tissue without being so traumatic as to cause slipping or undue tissue damage.

As described above with reference to block 9016d and block 9016f of process 9016 shown in FIG. 15, the process of delivering implant device 6002 may include at least two stages where contact member 6304 is retracted proximally between stages of rotating contact member 6304 within the LAA. As also described above, each of these proximal retractions of contact member 6304 may be along a respective range of motion (e.g., a range of anywhere from approximately 1 mm to approximately 15 mm; from approximately 1 mm to approximately 6 mm; or more specifically, from approximately 2 mm to approximately 3 mm). These stages of proximal retraction, including within the specific ranges of motion that are provided as illustrative examples above, may be beneficial to accommodate longitudinal bunching of the LAA that may tend to naturally occur as the LAA is twisted by contact member 6304 during the stages of rotating contact member 6304 as described above. In other words, when the LAA is twisted by contact member 6304 during the stages of rotating contact member 6304 as described above, such twisting may tend to induce tensile stress within the LAA, and the stages of proximal retraction of contact member 6304 with the LAA, including within the specific ranges of motion that are provided as illustrative examples above, may relieve such tensile stress and facilitate further twisting of the LAA. In the absence of these stages of proximal retraction of contact member 6304 with the LAA, the unrelieved tensile stress within the LAA may urge the LAA to untwist, may result in undue tissue damage, and/or may cause other undesirable results. Keeping the range of proximal movement during the stages of proximal retraction of contact member 6304 with the LAA within the ranges indicated above may minimize the risk of prolapsing contact member 6304 and adjacent portions of the LAA into the left atrium.

### V. Example of Alternative Securing Element

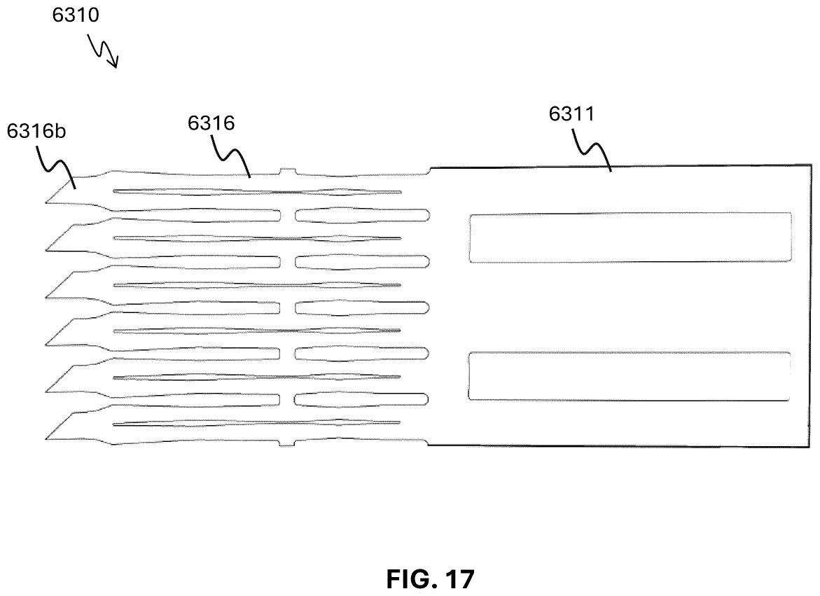

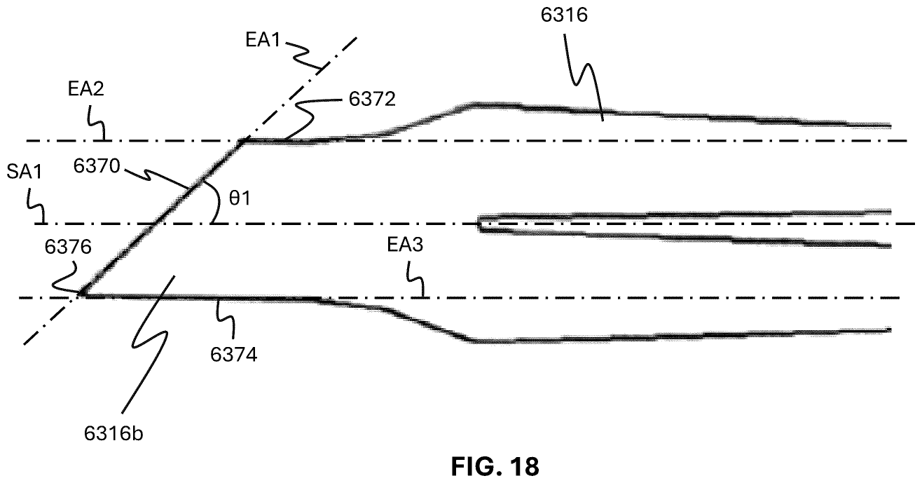

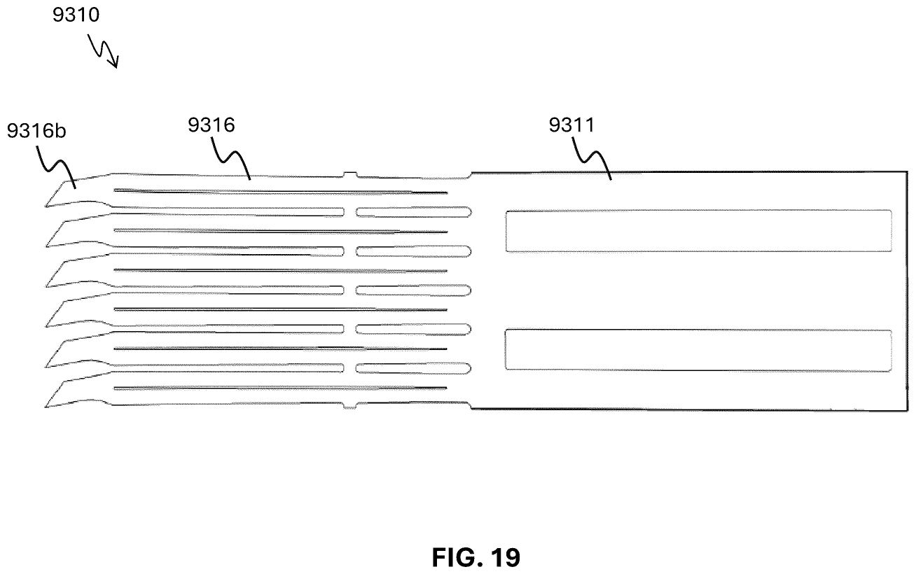

In some versions, the process of manufacturing securing element 6310 includes steps where securing element 6310 is in a flat, substantially two-dimensional shape. By way of example only, securing element 6310 may be initially formed through a process of cutting a sheet of material (e.g., nitinol) using any suitable technique(s). Securing element 6310 may then be wrapped about a central axis and secured in a three-dimensional shape as shown in FIGS. 4A-8A, 10A-10C, 11B-12D, 13C, and 16A-16C. FIG. 17 shows an example of what securing element 6310 may look like in a flat, substantially two-dimensional shape. As best seen in FIG. 18, each strut 6316 of securing element 6310 defines a respective strut axis SA1.

As also best seen in FIG. 18, distal end portion 6316b includes a distal edge 6370, a first lateral edge 6372, and a second lateral edge 6374. Edges 6370, 6374 converge distally to form a sharp tip 6376. Distal edge 6370 extends along a first edge axis EA1. First lateral edge 6372 extends along a second edge axis EA2. Second lateral edge 6374 extends along a third edge axis EA3. In the example shown, first edge axis EA1 forms an oblique angle Θ1 with strut axis SA1. By way of example only, angle Θ1 may range from approximately 30 degrees to approximately 60 degrees, from approximately 40 degrees to approximately 50 degrees; or may be approximately 45 degrees. By contrast, second edge axis EA2 and third edge axis EA3 are each parallel with strut axis SA1 and parallel with each other in this example.

In some scenarios (e.g., in cases where securing element 6310 is relatively large, such as having a width of approximately 18 mm; or when securing element 6310 is used with a contact member 6304 having a width of approximately 18 mm; or other scenarios), it may be desirable to modify the configuration of distal end portion 6316b to further reduce the risk of the LAA unwrapping, or the risk of any other kind of unintended tissue movement, when implant device 6302 is finally deployed in the patient. To that end, FIG. 19 shows an example of an alternative securing element 9310 that may be used as a substitute for securing element 6310. Securing element 9310 of this example may be configured and operable just like securing element 6310, except for the differences explicitly noted below. Thus, while FIG. 19 shows securing element 9310 in a flat, substantially two-dimensional shape, securing element 9310 may be wrapped about a central axis and secured in a three-dimensional shape resembling the shape shown in FIGS. 4A-8A, 10A-10C, 11B-12D, 13C, and 16A-16C. Like securing element 6310, securing element 9310 of this example includes a body portion 9311 and a set of struts 9316 extending distally from body portion 9311, with each strut 9316 having a respective distal end portion 9316b.

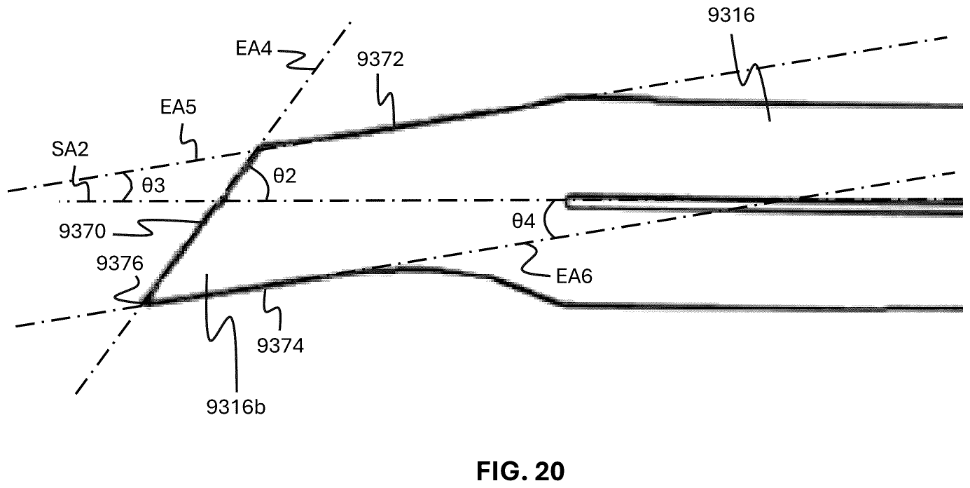

As best seen in FIG. 20, each strut 9316 of securing element 9310 defines a respective strut axis SA2. As also best seen in FIG. 20, distal end portion 9316b includes a distal edge 9370, a first lateral edge 9372, and a second lateral edge 9374. Edges 9370, 9374 converge distally to form a pointed tip 9376. Distal edge 9370 extends along a fourth edge axis EA4. First lateral edge 9372 extends along a fifth edge axis EA5. Second lateral edge 9374 extends along a sixth edge axis EA6. In the example shown, fourth edge axis EA4 forms an oblique angle Θ2 with strut axis SA2. By way of example only, angle Θ2 may range from approximately 30 degrees to approximately 60 degrees, from approximately 40 degrees to approximately 50 degrees; or may be approximately 45 degrees.

Unlike the configuration of distal end portion 6316b each strut 6316 of securing element 6310, distal end portion 9316b each strut 9316 of securing element 9310 has a swept configuration. Specifically, fifth edge axis EA5 forms an oblique angle Θ3 with strut axis SA2 rather than being parallel with strut axis SA2. Similarly, sixth edge axis EA6 forms an oblique angle Θ4 with strut axis SA2 rather than being parallel with strut axis SA2. However, fifth edge axis EA5 is parallel with sixth edge axis EA6 in this example, such that angle Θ3 is equal to angle Θ4 in this example. In some other versions, fifth edge axis EA5 is not parallel with sixth edge axis EA6, such that angle Θ3 need not necessarily be equal to angle Θ4 in all versions. By way of further example only, each angle Θ3, Θ4 may range from approximately 5 degrees to approximately 25 degrees, from approximately 10 degrees to approximately 20 degrees; or may be approximately 15 degrees.

The addition of angles Θ3, Θ4 to distal end portion 9316b may further reduce the risk of the LAA unwrapping, or the risk of any other kind of unintended tissue movement, when implant device 6302 is finally deployed in the patient, in some scenarios. For instance, the addition of angles Θ3, Θ4 to distal end portion 9316b may further reduce the risk of the LAA unwrapping, or the risk of any other kind of unintended tissue movement, when securing element 9310 is relatively large, such as having a width of approximately 18 mm. In addition, or in the alternative, the addition of angles Θ3, Θ4 to distal end portion 9316b may further reduce the risk of the LAA unwrapping, or the risk of any other kind of unintended tissue movement, when securing element 9310 is used with a contact member 6304 having a width of approximately 18 mm. Alternatively, there may be other scenarios where the addition of angles Θ3, Θ4 to distal end portion 9316b may further reduce the risk of the LAA unwrapping, or the risk of any other kind of unintended tissue movement, when implant device 6302 is finally deployed in the patient. In addition to reducing the risk of the LAA unwrapping, or the risk of any other kind of unintended tissue movement (or in lieu of reducing the risk of the LAA unwrapping, or the risk of any other kind of unintended tissue movement), the addition of angles Θ3, Θ4 to distal end portion 9316b may provide other beneficial performance results.

In some versions, each distal edge 9370 is oriented to urge the respective pointed tip 9376 into tissue in a clockwise direction when securing element 9310 is deployed. In some other versions, each distal edge 9370 is oriented to urge the respective pointed tip 9376 into tissue in a counterclockwise direction when securing element 9310 is deployed. In still other versions, securing element 9310 may be configured to have at least one strut 9316 with a distal end portion 9316b having a distal edge 9370 that is oriented to urge the respective pointed tip 9376 into tissue in a clockwise direction when securing element 9310 is deployed; and at least one other strut 9316 with a distal end portion 9316b having a distal edge 9370 that is oriented to urge the respective pointed tip 9376 into tissue in a counterclockwise direction when securing element 9310 is deployed. In some such cases, these differently oriented distal edges 9370 may be arranged on alternating struts 9316.

### VI. Miscellaneous

While certain arrangements of the inventions have been described, these arrangements have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, arrangement, or example are to be understood to be applicable to any other aspect, arrangement or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing arrangements. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some arrangements, the actual steps taken in the processes illustrated and/or disclosed may differ from those shown in the figures. Depending on the arrangement, certain of the steps described above may be removed, others may be added. Furthermore, the features and attributes of the specific arrangements disclosed above may be combined in different ways to form additional arrangements, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular arrangement. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain arrangements include, while other arrangements do not include, certain features, elements, and/or steps. Thus, such conditional language is not generally intended to imply that features, elements, and/or steps are in any way required for one or more arrangements or that one or more arrangements necessarily include logic for deciding, with or without user input or prompting, whether these features, elements, and/or steps are included or are to be performed in any particular arrangement.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain arrangements require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain arrangements, the terms “generally parallel” and “substantially parallel” refer to a value, amount, or characteristic that departs from exactly parallel by less than or equal to 15°, 10°, 5°, 3°, 1 degree, or 0.1 degree. The ranges disclosed herein also encompass any and all overlap, sub-ranges, and combinations thereof, and any specific values within those ranges. Language such as “up to,” “at least,” “greater than,” “less than,” “between,” and the like includes the number recited. Numbers and values used herein preceded by a term such as “about” or “approximately” include the recited numbers. For example, “approximately 7 mm” includes “7 mm” and numbers and ranges preceded by a term such as “about” or “approximately” should be interpreted as disclosing numbers and ranges with or without such a term in front of the number or value such that this application supports claiming the numbers, values and ranges disclosed in the specification and/or claims with or without the term such as “about” or “approximately” before such numbers, values or ranges such, for example, that “approximately two times to approximately five times” also includes the disclosure of the range of “two times to five times.” The scope of the present disclosure is not intended to be limited by the specific disclosures of preferred arrangements in this section or elsewhere in this specification, and may be defined by claims as presented in this section or elsewhere in this specification or as presented in the future. The language of the claims is to be interpreted broadly based on the language employed in the claims and not limited to the examples described in the present specification or during the prosecution of the application, which examples are to be construed as non-exclusive.

1. A method comprising:
(a) inserting a contact member into an anatomical cavity, the anatomical cavity being defined by tissue;
(b) rotating the contact member within the anatomical cavity through a first range of angular motion, the contact member engaging the tissue during rotation of the contact member within the anatomical cavity through the first range of angular motion such that rotation of the contact member within the anatomical cavity through the first range of angular motion causes torsional deformation of the anatomical cavity;
(c) moving the contact member longitudinally through a first range of longitudinal motion after rotating the contact member within the anatomical cavity through the first range of angular motion, thereby causing longitudinal deformation of the anatomical cavity; and
(d) securing a position of the contact member relative to the tissue, thereby maintaining the torsional and longitudinal deformation of the tissue.

2. The method of claim 1, the first range of angular motion having a value ranging from approximately 180 degrees to approximately 360 degrees.

3. The method of claim 1, the first range of angular motion being approximately 270 degrees.

4. The method of claim 1, the first range of longitudinal motion having a value ranging from approximately 1 mm to approximately 6 mm.

5. The method of claim 1, the first range of longitudinal motion having a value ranging from approximately 2 mm to approximately 3 mm.

6. The method of claim 1, the first range of longitudinal motion being along a longitudinal dimension, the contact member having a length along the longitudinal dimension, the first range of longitudinal motion being approximately equal to the length of the of the contact member along the longitudinal dimension.

7. The method of claim 1, further comprising rotating the contact member within the anatomical cavity through a second range of angular motion, the contact member engaging the tissue during rotation of the contact member within the anatomical cavity through the second range of angular motion such that rotation of the contact member within the anatomical cavity through the second range of angular motion causes further torsional deformation of the anatomical cavity.

8. The method of claim 7, the second range of angular motion having a value ranging from approximately 15 degrees to approximately 90 degrees.

8. (canceled)

9. The method of claim 7, further comprising moving the contact member longitudinally through a second range of longitudinal motion after rotating the contact member within the anatomical cavity through the second range of angular motion, thereby causing further longitudinal deformation of the anatomical cavity.

10. The method of claim 9, the second range of longitudinal motion having a value ranging from approximately 1 mm to approximately 6 mm.

11. The method of claim 9, the second range of longitudinal motion having a value ranging from approximately 2 mm to approximately 3 mm.

12. The method of claim 1, further comprising verifying closure of the anatomical cavity.

13. The method of claim 12, the act of verifying closure of the anatomical cavity comprising:
(i) injecting contrast medium near the anatomical cavity, and
(ii) observing the anatomical cavity under fluoroscopy to determine whether the injected contrast medium enters the anatomical cavity.

14. The method of claim 12, the act of inserting the contact member into the anatomical cavity including delivering the contact member via a catheter, the act of injecting the contrast medium comprising injecting the contrast medium via the catheter.

15. The method of claim 14, the catheter having a distal end with a plurality of slits, the act of injecting the contrast medium via the catheter comprising injecting the contrast medium via the plurality of slits.

16. The method of claim 12, further comprising rotating the contact member within the anatomical cavity through another range of angular motion after verifying closure of the anatomical cavity, before securing the position of the contact member relative to the tissue.

17. The method of claim 1, the act of securing a position of the contact member comprising delivering a securing element, the securing element engaging tissue external to the anatomical cavity while the contact member engages tissue within the anatomical cavity.

18. The method of claim 17, the contact member having an outer width, the securing element having an outer width, the ratio of the outer width of the contact member to the outer width of the securing element ranging from approximately 50% to approximately 175%.

19. A method comprising:
(a) measuring a left atrial appendage of a patient to obtain a measurement;
(b) selecting a left atrial appendage implant device from a plurality of left atrial appendage implant devices having varying sizes, the selection being made based at least in part on the measurement;
(c) preparing an implant delivery system, the act of preparing an implant delivery system comprising securing a delivery catheter to a support stand;
(d) advancing a distal end of the delivery catheter into the left atrium of the patient;
(e) delivering a first portion of the left atrial appendage implant device into the left atrial appendage via the delivery catheter;
(f) manipulating the first portion of the left atrial appendage implant device via the delivery catheter to thereby deform the left atrial appendage; and
(g) delivering a second portion of the left atrial appendage implant device via the delivery catheter to secure the position of the left atrial appendage implant device and to maintain the deformation of the left atrial appendage.

20. A method comprising:
(a) advancing a guide sheath into a patient to thereby position a distal end of the guide sheath near an anatomical cavity of the patient;
(b) securing the guide sheath to a support stand;
(c) advancing a delivery catheter through the guide sheath to thereby position a distal end of the delivery catheter near the anatomical cavity of the patient;
(d) securing the delivery catheter to the support stand;
(e) delivering a first portion of an implant device into the anatomical cavity via the delivery catheter;
(f) manipulating the first portion of the implant device via the delivery catheter to thereby deform the anatomical cavity; and
(g) delivering a second portion of the implant device via the delivery catheter to secure the position of the implant device and to maintain the deformation of the anatomical cavity.
